(19) United States
(12) Patent Application Publication
(10) Pub. No.: US 2025/0195632 A1
(43) Pub. Date: Jun. 19, 2025

(54) TOLERANCE-INDUCING CONSTRUCTS AND COMPOSITION AND THEIR USE FOR THE TREATMENT OF IMMUNE DISORDERS

(71) Applicant: Nykode Therapeutics ASA, Oslo (NO)

(72) Inventors: Agnete Brunsvik Fredriksen, Rælingen (NO); Heidi Myrset, Fetsund (NO)

(73) Assignee: Nykode Therapeutics ASA, Oslo (NO)

(21) Appl. No.: 18/560,272

(22) PCT Filed: May 10, 2022
(86) PCT No.: PCT/EP2022/062637

§ 371 Date: Nov. 10, 2023

(30) Foreign Application Priority Data
May 10, 2021 (DK) PA 2021 70222
Jul. 8, 2021 (DK) PA 2021 70367
Sep. 23, 2021 (EP) 21198526.2

(51) Int. Cl. A61K 39/00; A61K 39/35; A61K 39/36; A61P 37/06

(52) U.S. Cl. CPC A61K 39/0008; A61K 39/35; A61K 39/36; A61P 37/06; A61K 2039/577

(57) ABSTRACT

The present disclosure relates to constructs and compositions for use in the treatment of conditions involving undesired immune reactions, such as in the prophylactic or therapeutic treatment of autoimmune diseases, allergic disease and graft rejection.

### CROSS-REFERENCE TO RELATED APPLICATIONS

This application is a National Stage Entry of PCT/EP2022/062637, filed May 10, 2022, which claims the benefit of priority to EP Patent Application No. 21198526.2, filed Sep. 23, 2021, DK Patent Application No. 202170367, filed Jul. 8, 2021, and DK Patent Application No. 202170222, filed May 10, 2021, all of which are hereby incorporated by reference in their entirety.

### TECHNICAL FIELD

The present disclosure relates to constructs and compositions for use in the treatment of conditions involving undesirable immune reactions, such as in the prophylactic or therapeutic treatment of autoimmune diseases, allergic disease and graft rejection.

### REFERENCE TO AN ELECTRONIC SEQUENCE LISTING

The instant application contains a Sequence Listing which has been submitted in ASCII (ST.25) format via USPTO Patent Center, which is hereby incorporated by reference into the specification in its entirety. Said ASCII copy, named “P5967US00 Substitute Sequence listing.txt”, was created on Feb. 18, 2025, and is 210,889 bytes in size.

### BACKGROUND

Immune responses are necessary for protection against diseases, e.g. diseases caused by pathogens like viruses, bacteria or parasites. However, undesirable immune activation can cause processes leading to damage or destruction of one's own tissues. Undesirable immune activation occurs, for example, in autoimmune diseases where antibodies and/or T lymphocytes react with self-antigens resulting in e.g. tissue damage and pathology. Undesirable immune activation also occurs in allergic reactions, which are characterized by an exaggerated immune response to typically harmless substances in the environment and which may result in inflammatory responses leading to tissue destruction. Further, undesired immune activation occurs in graft rejection, e.g. rejection of transplanted organs or tissue which is significantly mediated by alloreactive T cells present in the host which recognize donor alloantigens or xenoantigens and which leads to destruction of the transplanted organ or tissue.

Immune tolerance is the acquired lack of specific immune responses to substances or tissue that have the capacity to elicit an immune response in a given organism.

Typically, to induce tolerance to a specific antigen, the antigen must be presented by antigen-presenting cells (APCs) to other immune cells in the absence of activation signals, which results in the death or functional inactivation of antigen-specific lymphocytes or the generation of antigen-specific cells that maintain the tolerance. This process generally accounts for tolerance to self-antigens, or self-tolerance. Immunosuppressive drugs are useful in prevention or reduction of undesirable immune responses, e.g., in treating patients with autoimmune diseases or with allogeneic transplants.

Conventional strategies for generating immunosuppression of an unwanted immune response are based on broad-acting immunosuppressive drugs. Additionally, to maintain immunosuppression, immunosuppressive drug therapy is often a life-long proposition. Unfortunately, the use of broad-acting immunosuppressive drugs is associated with a risk of severe side effects, such as immunodeficiency, because most of them act non-selectively, resulting in increased susceptibility to infections and decreased cancer immunosurveillance. Accordingly, new compounds and compositions that induce antigen-specific tolerance would be beneficial.

APCs, such as dendritic cells play a key role in regulating the immune response, and, depending on the activation state and the microenvironment of the dendritic cell (cytokines and growth factors), it gives the antigen-specific T cells signal to either combat the presented antigens (presumed pathogens) or to silence the reaction to the presented antigens (presumed non-pathogenic antigens) and induce peripheral tolerance. The challenge in developing tolerogenic immunotherapies is to efficiently deliver the antigen to the APCs, such as dendritic cells, in a manner that does not trigger inflammation or an immune response, such as an inflammatory immune response.

The scientific article “Schjetne K W et al., Eur. J. Immunol. 35(11), 3142-3152, 2005” discloses recombinant antibody constructs, referred to as “Troybodies”. These Troybodies are recombinant antibodies with V regions specific for APC surface molecules and T cell epitopes engrafted in the loops between β-strands in their C domains.

### SUMMARY

The present disclosure relates to tolerance-inducing constructs that comprise an antigen unit and a targeting unit that interacts with surface molecules on APCs, such as dendritic cells, in a non-inflammatory or tolerogenic manner which leads to the presentation of the antigen in the absence of activation, such as an inflammatory activation.

The present inventors have surprisingly found that the Vaccibody platform can deliver disease-relevant antigens to antigen-presenting cells (APCs) in an optimal way for the induction of an antigen-specific tolerance response of choice, through binding to and signalling through selected surface receptors on APCs that internalize the construct and present the antigens in a tolerance inducing manner, such as induction of regulatory T cells (Tregs) and suppression of memory and effector T cell responses.

The tolerance-inducing constructs of the disclosure may have improved flexibility compared to known constructs, such as the “Troybodies” disclosed in Schjetne K W et al., Eur. J. Immunol. 35(11), 3142-3152, 2005. For instance, the targeting unit of the disclosed constructs is not limited to antibody-derived V regions, but can be a wide variety of different units.

A further advantage of the tolerance-inducing constructs of the disclosure compared to known constructs may be that fewer doses, such as one dose, may be sufficient to reach the same functional effect. For instance, fewer doses, such as one dose, may be sufficient to decrease the level of an immune response, delay the onset or progression of an immune response and/or reduce the risk of the onset or progression of an immune response.

The Vaccibody construct is a multimeric protein consisting of multiple polypeptides, for example, a dimeric protein consisting of two polypeptides, each comprising a targeting unit, which targets antigen-presenting cells, a dimerization unit and an antigenic unit—see for example WO 2004/076489 A1, WO 2011/161244 A1, WO 2013/092875 A1 or WO 2017/118695 A1. These constructs have shown to be efficient in generating an immune response against the antigens or epitopes comprised in the antigenic unit.

The Vaccibody or the tolerance-inducing construct of the present disclosure may be administered to a subject in the form of a polynucleotide (e.g. a DNA plasmid) comprising a nucleotide sequence encoding the polypeptide. After administration to host cells, e.g. muscle cells of a human, the polypeptide is expressed and, due to the multimerization unit, forms a multimeric protein; when a dimerization unit is used, the polypeptide when expressed forms a dimeric protein.

The present disclosure provides tolerance-inducing constructs based on a Vaccibody structure for use in the prophylactic or therapeutic treatment of immune diseases such as autoimmune diseases, allergic disease and graft rejection.

The tolerance-inducing constructs of the disclosure comprise an antigenic unit that comprises one or more T cell epitopes of a self-antigen, allergen or alloantigen/xenoantigen, a multimerization unit, for example a dimerization unit, and a targeting unit that targets APCs. The targeting unit interacts with surface molecules on the APC in such a way that the construct is internalized and the epitopes in the antigenic unit are presented in a tolerance-inducing manner.

Thus, in a first aspect, the disclosure provides a tolerance-inducing construct comprising:
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides as defined in (ii), such as two polypeptides;
   wherein the antigenic unit comprises one or more T cell epitopes of a self-antigen, an allergen, an alloantigen or a xenoantigen.

In another aspect, the present disclosure provides a tolerance-inducing construct comprising:
i) a polynucleotide comprising a nucleotide sequence encoding a targeting unit targeting or capable of targeting antigen-presenting cells, a multimerization unit, and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In another aspect, the present disclosure provides a tolerance-inducing construct comprising:
i) a polynucleotide comprising a nucleotide sequence encoding a targeting unit targeting or capable of targeting antigen-presenting cells, a dimerization unit, and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

In another aspect, the disclosure provides a polynucleotide as defined herein.

In another aspect, the disclosure provides a vector comprising the polynucleotide as defined herein.

In another aspect, the disclosure provides a host cell comprising the polynucleotide as defined herein.

In another aspect, the disclosure provides a dimeric protein consisting of two polypeptides as defined herein.

In another aspect, the disclosure provides a polypeptide encoded by the nucleic acid as defined herein.

In another aspect, the disclosure provides a pharmaceutical composition comprising the tolerance-inducing construct as defined herein and a pharmaceutically acceptable carrier.

In another aspect, the disclosure provides a method for preparing the pharmaceutical composition as defined herein, wherein the pharmaceutical composition comprises the polypeptide as defined herein or the multimeric such as a dimeric protein as defined herein, wherein the method comprises:
a) transfecting cells with the polynucleotide as defined herein;
b) culturing the cells;
c) collecting and purifying the multimeric protein, such as the dimeric protein, or the polypeptide expressed from the cells; and
d) mixing the multimeric protein, such as the dimeric protein, or the polypeptide obtained from step c) with a pharmaceutically acceptable carrier.

In another aspect, the disclosure provides a method for preparing the pharmaceutical composition as defined herein, wherein the pharmaceutical composition comprises the polynucleotide as defined herein, the method comprises:
a) preparing the polynucleotide;
b) optionally cloning the polynucleotide into an expression vector; and
c) mixing the polynucleotide obtained from step a) or the vector obtained from step b) with the pharmaceutically acceptable carrier.

In another aspect, the disclosure provides a method for treating a subject suffering from a condition involving undesirable immune reactions, such an autoimmune disease, allergic disease or graft rejection, or being in need of prevention thereof, the method comprising administering to the subject the pharmaceutical composition as defined in herein.

In another aspect, the disclosure provides a pharmaceutical composition as defined herein for use in the treatment of a condition involving undesirable immune reactions, such an autoimmune disease, allergic disease or graft rejection.

### DESCRIPTION OF DRAWINGS

FIG. 1: Schematic drawing of an exemplary tolerance-inducing construct

FIG. 1 shows an example of a tolerance-inducing construct of the disclosure. The tolerance-inducing construct of the disclosure can be described as a polypeptide having an N-terminal start and a C-terminal end (illustrated in FIG. 1). The elements of the polypeptide—targeting unit (TU), dimerization unit (DimU) and antigenic unit—may be arranged in the polypeptide such that the antigenic unit is located at the C-terminal end of the polypeptide (FIG. 1a) or at the N-terminal start of the polypeptide (FIG. 1b).

The antigenic unit comprises one or more T cell epitope(s) and, if multiple T cell epitopes are present, may comprise one or more T cell epitope linkers. A unit linker (UL) may connect the dimerization unit and the antigenic unit. FIG. 1 illustrates an antigenic unit with 2 T cell epitopes (T1, T2), which are separated by a T cell epitope linker (TL). The order and orientation of the above-described units and elements are the same in the multimeric protein, the dimeric protein and the polynucleotide.

FIG. 2: Expression and secretion of MOG-containing tolerance-inducing constructs

FIGS. 2A and 2B show protein expression and secretion levels of MOG-containing tolerance-inducing constructs and the pro-inflammatory control constructs VB5002b and VB5052, detected by sandwich ELISA (capture antibody: anti-MOG antibody, detection antibody: anti-hlgG CH3 domain antibody) (A) with supernatant from HEK293 cells transiently transfected with the DNA vectors VB5002b, VB5003b, VB5004b, VB5005b, VB5006b and VB5012b, (B) with supernatant from Expi293F cells transiently transfected with the DNA vectors VB5052, VB5046, VB5048, VB5058, VB5059, VB5060, VB5061 and VB5071. All the MOG-containing constructs were highly expressed and secreted. The negative control in (A) is supernatant from HEK293 cells treated with the transfection reagent Lipofectamine only and in (B) supernatant form Expi293F cells treated with the transfection reagent ExpiFectamine only.

FIG. 3: Secretion of full-length MOG-containing tolerance-inducing constructs with different targeting unit.

FIG. 3 shows high level secretion of full-length tolerance-inducing constructs, and the pro-inflammatory control construct VB5052, with different targeting unit as detected by sandwich ELISA of supernatants from HEK293 cells or Expi293F cells transiently transfected with the vectors VB5005b, VB5006b (HEK293 cells), VB5052, VB5058, VB5059, VB5060 and VB5061 (Expi293F cells). Capture antibody: mouse anti-MOG antibody, 0.25 μg/ml, 100 μl/well, sc-73330, Santa Cruz Biotechnology. Detection antibody: (A) and (B) 0.2 μg/ml goat anti-murine IL-10 biotinylated antibody, 100 μl/well, BAF417, R&D Systems (C) 0.8 μg/ml chicken anti-human TGF-beta 1 Biotinylated Antibody, 100 μl/well, BAF240, RD Systems. (D) 0.83 μg/ml goat anti-murine SCGB3A2 biotinylated antibody, 100 μl/well, BAF3465, R&D Systems. (E) 0.8 μg/ml goat anti-murine CTLA-4 biotinylated antibody, 100 μl/well, BAF476, RD Systems. (F) 0.29 μg/ml goat anti-mouse PD-1 biotinylated antibody, 100 μl/well, DY1021, R&D System. (G) 0.2 μg/ml goat anti-human CCL3 biotinylated antibody, 100 μl/well, BAF270, R&D Systems. The negative control is supernatant from HEK293 cells treated with the transfection reagent Lipofectamine only or supernatant from Expi293F cells treated with ExpiFectamine only.

FIG. 4: Secretion of the MOG(27-63) peptide

The secretion of the MOG(27-63) peptide encoded in the DNA vector VB5051 was verified by direct ELISA (detection antibody: mouse anti-MOG antibody, 3.3 μg/ml, 100 μl/well, sc-73330, Santa Cruz Biotechnology) of supernatant from Expi293F cells transiently transfected with the DNA vector VB5051. The negative control is supernatant from Expi293F cells treated with the transfection reagent Expifectamine only.

FIG. 5: Expression and secretion of Met e 1 containing tolerance-inducing constructs

FIG. 5 shows the protein expression and secretion levels of Met e 1 containing tolerance-inducing constructs detected by sandwich ELISA (capture antibody: anti-human IgG3 (CH3 domain) antibody, detection antibody: CaptureSelect™ Biotin Anti-IgG-Fc (Human) Conjugate) of supernatant from Expi293F cells transiently transfected with the Met e 1-containing DNA vectors VB5024, VB5030 and VB5079. All the Met e 1 containing tolerance-inducing constructs were expressed and secreted. The negative control is supernatant from Expi293F cells treated with the transfection reagent Expifectamine only.

FIG. 6: Tolerance-inducing constructs comprising scFv anti-DEC205 as targeting unit binds to recombinant DEC205 receptor

FIG. 6 shows that tolerance-inducing proteins comprising a scFv anti-DEC205 targeting unit binds recombinant DEC205 receptor by direct ELISA (coat: recombinant DEC205(216-503), detection antibody: anti-MOG antibody or anti-hlgG CH3 domain antibody) of supernatant from HEK293 cells transiently transfected with the scFv anti-DEC205-containing DNA vector VB5004b. Binding to the receptor was confirmed by both antibodies, and the anti-MOG antibody confirmed the secretion of the full-length protein.

FIG. 7: Tolerance-inducing constructs comprising IL-10 as targeting unit binds to recombinant IL-10 receptor

FIG. 7 shows that tolerance-inducing proteins comprising IL-10 as targeting unit bind recombinant IL-10 receptor by direct ELISA (coat: recombinant IL-10 receptor, detection antibody: anti-MOG antibody or anti-hlgG CH3 domain antibody) of supernatant from HEK293 cells transiently transfected with the IL-10 containing DNA vector, VB5006b. Binding to the receptor was confirmed by both antibodies, and the anti-MOG antibody confirmed the secretion of the full-length protein.

FIG. 8: Characterization of size, protein integrity and dimer formation of secreted tolerance-inducing constructs

FIG. 8 shows Western blot (WB) analysis of supernatant from Expi293F cells transiently transfected with MOG-containing DNA vectors under reducing and non-reducing conditions. The negative control is supernatant from Expi293F cells treated with the transfection reagent ExpiFectamine (transfection control).

FIG. 8A: Western blot shows expression and full-length secretion of tolerance-inducing proteins. Reduced supernatant samples (25 μL loaded) from transfected Expi293F cells. Primary antibody: Mouse anti-MOG (sc-73330). Secondary antibody: Donkey anti-mouse, Dylight 800 (SA5-10172). Protein standard was detected in Chemidoc channel Dylight 650 (signal not shown) and Chemidoc channel Dylight 800.

FIG. 8B: Western blot shows dimerization of tolerance-inducing proteins (black arrows). Non-reduced supernatant samples (25 μL loaded) from transfected Expi293F cells. Primary antibody: Mouse anti-MOG (sc-73330). Secondary antibody: Donkey anti-mouse, Dylight 800 (SA5-10172). Chemidoc channels Dylight 650 (for protein standard) and 800.

FIG. 8C: Western blot shows expression and full-length secretion of tolerance-inducing proteins. Reduced supernatant samples (25 μL loaded) from transfected Expi293F cells. Primary antibody: Rat anti-IL10 (MAB417). Secondary antibody: Donkey anti-rat, Dylight 488 (SA5-10026). Chemidoc channels Dylight 650 (for protein standard) and 488.

FIG. 8D: Western blot shows expression and full-length secretion of tolerance-inducing proteins (black arrow). Reduced supernatant samples (35 μL loaded) from transfected Expi293F cells. Primary antibody: Goat anti-CTLA-4 (AF476). Secondary antibody: Donkey anti-goat, Dylight 800 (SA5-10092). Chemidoc channels Dylight 650 (for protein standard) and 800.

FIG. 9. Dual colour IL-10/IFNγ FluoroSpot.

C57BL/6 mice were vaccinated once (day 0) with 50 μg of indicated DNA vectors (VB5004b, VB5002b and VB5001b), and spleens were harvested at day 7 post vaccination. (A) Splenocytes of mice tested for IFN-γ and IL-10 secretion (SFU/106 splenocytes) with dual color FluoroSpot upon restimulation with MOG(35-55) peptide. Individual mice are shown, n=4 (VB5001b), 5 (VB5004b and VB5002b) or 2 (PBS) per group. (B) IL-10/IFN-γ ratios are plotted from data in (A). Individual mice and mean±range are shown, *(p<0.05) **(p<0.01), two-tailed Mann-Whitney test.

FIG. 10. Detection of % Foxp3+, % IFN-γ and % IL-17 producing CD4+ T cells by flow cytometry.

C57BL/6 mice were vaccinated once (day 0) with 50 μg of indicated DNA vectors (VB5004b, VB5002b, VB5001b), and spleens were harvested at day 7 post vaccination. Percentages [%] of (A) Foxp3+, (B) IFN-γ+ and (C) IL-17+ splenocytes among the total CD4+ T cell population upon restimulation with MOG(35-55) peptide are shown. Data were acquired from pools of 4 (VB5001b), 5 (VB5004b and VB5002b) or 2 (PBS) mice/group. Construct ID numbers are indicated at the x-axes.

FIG. 11. Dual color IL-10/IFNγ FluoroSpot.

C57BL/6 mice were vaccinated twice (day 0 and day 4) with 50 μg of indicated DNA vectors (VB5012b, VB5052, VB5051), and spleens were harvested at day 10 post prime vaccination. (A) Splenocytes of mice tested for IFN-γ and IL-10 secretion (SFU/106 splenocytes) with dual color FluoroSpot upon restimulation with MOG(35-55) peptide. Individual mice are shown. (B) IL-10/IFN-γ ratios are plotted from data in (A). Individual mice and mean±range are shown, n=5 or n=2 (PBS) per group, **(p<0.01), two-tailed Mann-Whitney test.

FIG. 12. Detection of MOG(38-49) specific Foxp3+ T cell.

C57BL/6 mice were vaccinated twice (day 0 and day 4) with 50 μg of indicated DNA vectors (VB5012b, VB5048, VB5006b, VB5046, VB5051), and spleens were harvested at day 10 post prime vaccination. Percentage of splenic Foxp3+ cells detected ex vivo by H-2 lab/MOG(38-49) tetramers of the total CD4+ population. Data were acquired from pools of 5 mice or 2 mice (PBS) per group. Construct ID numbers are indicated on the x-axis.

FIG. 13: Expression and secretion of a MOG-containing tolerance-inducing construct

FIG. 13 shows the protein expression and secretion level of the MOG-containing tolerance-inducing construct VB5009 with TGFβ1 as targeting unit—as detected by sandwich ELISA (capture antibody: rabbit anti human TGFβ1 (orb77216, Biorbyte), detection antibody: biotinylated mouse anti-human IgG (05-4240, Invitrogen) of supernatant from HEK293 cells transiently transfected with the VB5009. The negative control is supernatant from HEK293 cells treated with the transfection reagent Lipofectamine only.

### DETAILED DESCRIPTION

Thus, in a first aspect, the present disclosure provides a tolerance-inducing construct comprising:
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides as defined in (ii), such as of two polypeptides;
   wherein the antigenic unit comprises one or more T cell epitopes of a self-antigen, an allergen, an alloantigen or a xenoantigen.

In another aspect, the present disclosure provides a tolerance-inducing construct comprising:
i) a polynucleotide comprising a nucleotide sequence encoding a targeting unit targeting or capable of targeting antigen-presenting cells, a multimerization unit, and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In another aspect, the present disclosure provides a tolerance-inducing construct comprising:
i) a polynucleotide comprising a nucleotide sequence encoding a targeting unit targeting or capable of targeting antigen-presenting cells, a dimerization unit, and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

Such a construct will, once administered to a subject, allow the presentation of the epitopes in the antigenic unit in a tolerance-inducing manner and is thus suitable for use as a prophylactic or therapeutic treatment of immune diseases such as autoimmune diseases, allergic disease and graft rejection.

As the tolerance-inducing construct causes downregulation of the disease-specific cells of the immune system causing the immune disease in question, it will not suppress the general immune system. Thus, treatment of the immune disease in question with the construct of the disclosure will therefore not result in increased susceptibility to infections and decreased cancer immunosurveillance. However, bystander suppression of immune cells specific for related disease antigens are expected, due to the release of short-range inhibitory cytokines by cell-to-cell contact with the induced antigen-specific regulatory cells.

The tolerance-inducing construct of the disclosure may be administered in the form of a pharmaceutical composition comprising the construct of the disclosure and a pharmaceutically acceptable carrier, for use in the prophylactic or therapeutic treatment of immune disease such as autoimmune diseases, allergic disease and graft rejection.

A “nucleotide sequence” is a sequence consisting of nucleotides. The terms “nucleotide sequence” and “nucleic acid sequence” are used interchangeably herein

A “tolerance-inducing construct” is one that does not elicit an immune response, such as an inflammatory immune response, but rather does induce tolerance towards the T cell epitopes comprised in the antigenic unit, when administered to a subject in a form suitable for administration and in an amount effective to induce tolerance (i.e. an effective amount).

The term “tolerance” as used herein refers to a decreased level of an immune response, such as an inflammatory immune response, a delay in the onset or progression of an immune response, such as an inflammatory immune response, and/or a reduced risk of the onset or progression of an immune response, such as an inflammatory immune response.

A “subject” is an animal or human. A subject may be a patient, i.e. a human suffering from an immune disease like an autoimmune disease, an allergy or a graft rejection, who is in need of a therapeutic treatment. The terms “subject” and “individual” are used interchangeably herein.

A “disease” is an abnormal medical condition that is typically associated with specific signs and symptoms in a subject being affected by the disease.

An “immune disease” as used herein refers to conditions, disorders or diseases involving undesired immune reactions, including autoimmune diseases, allergies or a graft rejection, i.e. rejection of allografts or xenografts such as rejection by a host of cells, tissue or organs from the same (allo) or a different (xeno) species transplanted to the host.

The term “alloantigen” or “allograft antigen” as used herein refers to an antigen derived from (shed from and/or present in) a cell or tissue which, when transferred from a donor to a recipient, can be recognized and bound by an antibody of B or T-cell receptor of the recipient. Alloantigens are typically products of polymorphic genes. An alloantigen is a protein or peptide which, when compared between donor and recipient (belonging to the same species), displays slight structural differences. The presence of such a donor antigen in the body of a recipient can elicit an immune response in the recipient. Such alloreactive immune response is specific for the alloantigen.

The terms “murine” and “mouse” are used interchangeably to refer to a substance, such as a peptide, protein, nucleic acid, etc., derived from a mouse.

The term “xenoantigen” as used herein refers to an antigen derived from an individual of a different species.

A “treatment” is a prophylactic treatment or therapeutic treatment.

A “prophylactic treatment” is a treatment administered to a subject who does not display signs or symptoms of, or displays only early signs or symptoms of, an immune disease, such that treatment is administered for the purpose of preventing or at least decreasing the risk of developing the disease. A prophylactic treatment functions as a preventative treatment against an immune disease, or as a treatment that inhibits or reduces further development or enhancement of the immune disease and/or its associated symptoms. The terms “prophylactic treatment”, “prophylaxis” and “prevention” are used interchangeably herein.

A “therapeutic treatment” is a treatment administered to a subject who displays symptoms or signs of an immune disease, in which treatment is administered to the subject for the purpose of diminishing or eliminating those signs or symptoms and/or for the purpose of delaying or stopping disease progression.

A “part” refers to a part or fragment of an antigen, i.e. part or fragment of the amino acid sequence of an antigen, or the nucleotide sequence encoding same, e.g. an epitope; preferably, the part or fragment of the antigen is immunogenic. These terms will be used throughout interchangeably.

A “T cell epitope” as used herein refers to a single T cell epitope or a part or region of an antigen containing multiple T cell epitopes, e.g. multiple minimal epitopes.

The terms “vaccination” and “administration” are used interchangeably herein.

The term “minimal epitope” refers to a subsequence of an epitope predicted to bind to MHC I or MHC II. In other words, the minimal epitope may be immunogenic, i.e. capable of eliciting an immune response. The term minimal epitope thus may refer to short subsequences of an epitope, which are predicted to bind to MHC I or MHC II. A 27-mer epitope may thus encompass several minimal epitopes, which may each have a length shorter than 27 amino acids, and which each are immunogenic. For example, a minimal epitope could consist of the first 14 amino acids of the epitope, provided that it is predicted to bind to MHC I or MHC II, or it could consist of amino acids 9 to 18 of the epitope, or of amino acids 7 to 22, provided that these sequences are predicted to bind to MHC I or MHC II.

The section headings used herein are for organizational purposes only and are not to be construed as limiting the subject matter described.

### Tolerance-Inducing Construct

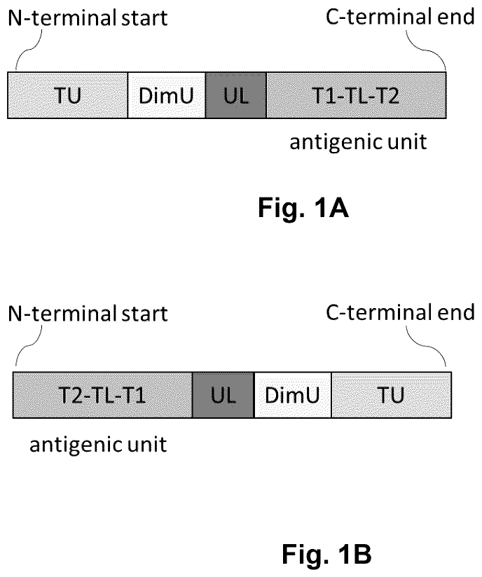

FIG. 1 shows an example of a tolerance-inducing construct of the disclosure. The tolerance-inducing construct of the disclosure can be described as a polypeptide having an N-terminal start and a C-terminal end (illustrated in FIG. 1). The elements of the polypeptide—targeting unit (TU), dimerization unit (DimU) and antigenic unit—may be arranged in the polypeptide such that the antigenic unit is located at the C-terminal end of the polypeptide (FIG. 1a) or at the N-terminal start of the polypeptide (FIG. 1b). Preferably, the antigenic unit is located at the C-terminal end of the polypeptide.

The antigenic unit comprises one or more T cell epitope(s) and, if multiple T cell epitopes are present, may comprise one or more T cell epitope linkers (TL). A unit liner (UL) may connect the dimerization unit and the antigenic unit. FIG. 1 illustrates an antigenic unit with 2 T cell epitopes (T1, T2), which are separated by a TL. The order and orientation of the above-described units and elements is the same in the /dimeric protein and the polynucleotide.

In the following, the various units of the construct will be discussed in detail. These units are present in the polynucleotide as nucleic acid sequences encoding the units while they are present in the polypeptide, multimeric protein or dimeric protein as amino acids sequences. For the ease of reading, in the following, the units of the construct are mainly explained in relation to the polypeptide, multimeric protein or dimeric protein, i.e. on the basis of their amino acid sequences.

### Targeting Unit

The tolerance-inducing construct of the disclosure comprises a targeting unit that targets antigen-presenting cells (APCs).

The term “targeting unit” as used herein refers to a unit that delivers the construct of the disclosure to an APC and interacts with surface molecules on the APC, e.g. binds to surface receptors on the APC, without activating the cell and/or without inducing maturation of the cell. The APC internalizes the construct and presents the T cell epitopes comprised in the antigenic unit on MHC on its surface in an anti-inflammatory, tolerogenic manner, e.g. by not upregulating co-stimulatory signals and/or upregulating inhibitory surface receptors and/or secretion of inhibitory cytokines.

In some embodiments, the targeting unit comprises or consists of a moiety that binds to a receptor selected from the group consisting of TGFβ receptor, such as TGFβR1, TGFβR2, or TGFβR3, IL10R, such as IL-10RA and IL10-RB, IL2R, IL4R, IL6R, IL11R and IL13R, IL27R, IL35R, IL37R, GM-CSFR, FLT3, CCR7, CD11b, CD11c, CD103, CD14, CD36, CD205, CD109, VISTA, MARCO, MHCII, CD83, SIGLEC, MGL/Clec10A, ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163 and CD141.

In some embodiments, the targeting unit comprises or consists of a moiety that binds to a human (h) receptor selected from the group consisting of hTGFβ receptor, such as hTGFβR1, hTGFβR2, or hTGFβR3, hIL10R, such as hIL-10RA and hIL10-RB, hIL2R, hIL4R, hIL6R, hIL11R and hIL13R, hIL27R, hIL35R, hIL37R, hGM-CSFR, hFLT3, hCCR7, hCD11b, hCD11c, hCD103, hCD14, hCD36, hCD205, hCD109, hVISTA, hMARCO, hMHCII, hCD83, hSIGLEC, hMGL/hClec10A, hASGR (hASGR1/hASGR2), hCD80, hCD86, hClec9A, hClec12A, hClec12B, hDCIR2, hLangerin, hMR, hDC-Sign, hTreml4, hDectin-1, hPDL1, hPDL2, hHVEM, hCD163 and hCD141.

The moiety may be a natural ligand, an antibody or part thereof, e.g. a scFv, or a synthetic ligand.

In some embodiments, the moiety is an antibody or part thereof, e.g. a scFv, with specificity for any of the aforementioned receptors, whose binding to the receptor results in the antigen and/or T cell epitopes being presented in an anti-inflammatory, tolerogenic manner.

In other embodiments, the moiety is a synthetic ligand with specificity for any of the aforementioned receptors, where binding to the receptor results in the antigen and/or T cell epitopes being presented in an anti-inflammatory, tolerogenic manner. Protein modelling may be used to design such synthetic ligands.

In other embodiments, the moiety is a natural ligand.

In some embodiments, natural ligand is selected from the group consisting of TGFβ, such as TGFβ1, TGFβ2 orTGFβ3, IL-10, IL2, IL4, IL6, IL11, IL13, IL27, IL35, IL37, GM-CSF, FLT3L, CCL19, CCL21, ICAM-1 (Intercellular Adhesion Molecule 1 also known as CD54), keratin, VSIG-3, SCGB3A2, CTLA-4, preferably the extracellular domain of CTLA-4, PD-1, preferably the extracellular domain of PD-1 and BTLA, preferably the extracellular domain of BTLA.

In other embodiments, the targeting unit is or comprises IL2, preferably human IL2. In other embodiments, the targeting unit comprises or consists of an amino acid sequence having at least 80% sequence identity to that of human IL2, such as an amino acid sequence having at least 80% sequence identity to SEQ ID NO: 33. In other embodiments, the targeting unit comprises or consists of or a nucleotide sequence encoding human IL2, such as the nucleotide sequence of SEQ ID NO: 36.

In other embodiments, the targeting unit is or comprises IL-10 or TGFβ, preferably human IL-10 or human TGFβ, including its isoforms TGFβ-1, TGFβ-2 and TGFβ-3.

In other embodiments, the targeting unit comprises or consists of an amino acid sequence having at least 80% sequence identity to that of human TGFβ, such as an amino acid sequence having at least 80% sequence identity to any of SEQ ID NO: 205-207.

In yet other embodiments, the targeting unit comprises or consists of an amino acid sequence having at least 85% sequence identity to the amino acid sequence of human TGFβ, such as an amino acid sequence having at least 85% sequence identity to any of SEQ ID NO: 205-207, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In another embodiment, the targeting unit comprises or consists of an amino acid sequence of human TGFβ, such as an amino acid sequence selected from SEQ ID NO: 205-207, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human TGFβ, or a nucleotide sequence encoding human TGFβ.

In other embodiments, the targeting unit comprises or consists of or a nucleotide sequence encoding human TGFβ, such as a nucleotide sequence selected from SEQ ID NO: 208-210.

In yet other embodiments, the targeting unit comprises or consists of an amino acid sequence having at least 80% sequence identity to that of murine TGFβ, such as murine TGFβ as set forth in SEQ ID NO: 177.

In yet other embodiments, the targeting unit comprises or consists of an amino acid sequence having at least 80% sequence identity to that of human IL-10, such as an amino acid sequence having at least 80% sequence identity to SEQ ID NO: 211.

In yet other embodiments, the targeting unit comprises or consists of an amino acid sequence having at least 85% sequence identity to the amino acid sequence of human IL-10, such as an amino acid sequence having at least 85% sequence identity to SEQ ID NO: 211, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human IL-10, such as the amino acid sequence of SEQ ID NO: 211, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human IL-10, or a nucleotide sequence encoding human IL-10.

In other embodiments, the targeting unit comprises or consists of or a nucleotide sequence encoding human IL-10, such as the nucleotide sequence of SEQ ID NO: 212.

In yet other embodiments, the targeting unit comprises or consists of an amino acid sequence having at least 80% sequence identity to that of murine IL-10, such as murine IL-10 as set forth in SEQ ID NO: 169.

In some embodiments, the targeting unit is or comprises SCGB3A2 or VSIG-3, preferably human VSIG-3 or human SCGB3A2.

In other embodiments, the targeting unit comprises or consists of an amino acid sequence having at least 80% sequence identity to that of human SCGB3A2, such as an amino acid sequence having at least 80% sequence identity to SEQ ID NO: 213.

In yet other embodiments, the targeting unit comprises or consists of an amino acid sequence having at least 85% sequence identity to the amino acid sequence of human SCGB3A2, such as an amino acid sequence having at least 85% sequence identity to SEQ ID NO: 213, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human SCGB3A2, such as the amino acid sequence of SEQ ID NO: 213, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human SCGB3A2, or a nucleotide sequence encoding human SCGB3A2.

In other embodiments, the targeting unit comprises or consists of or a nucleotide sequence encoding human SCGB3A2, such as the nucleotide sequence of SEQ ID NO: 214.

In yet other embodiments, the targeting unit comprises or consists of an amino acid sequence having at least 80% sequence identity to that of murine SCGB3A2, such as murine SCGB3A2 as set forth in SEQ ID NO: 171.

In yet other embodiments, the targeting unit comprises or consists of an amino acid sequence having at least 80% sequence identity to that of human VSIG-3, such as an amino acid sequence having at least 80% sequence identity to SEQ ID NO: 215.

In yet other embodiments, the targeting unit comprises or consists of an amino acid sequence having at least 85% sequence identity to the amino acid sequence of human VSIG-3, such as an amino acid sequence having at least 85% sequence identity to SEQ ID NO: 215, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In another embodiment, the targeting unit comprises or consists of an amino acid sequence of human VSIG-3, such as the amino acid sequence of SEQ ID NO: 215, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human VSIG-3, or a nucleotide sequence encoding human VSIG-3.

In other embodiments, the targeting unit comprises or consists of or a nucleotide sequence encoding human VSIG-3, such as the nucleotide sequence of SEQ ID NO: 216.

In yet other embodiments, the targeting unit comprises or consists of an amino acid sequence having at least 80% sequence identity to that of murine VSIG-3, such as murine VSIG-3 as set forth in SEQ ID NO: 173.

In yet other embodiments, the targeting unit is or comprises an antibody or part thereof, e.g. a scFv, with specificity for CD205, such as scFv with specificity for human or murine CD205 or an scFv anti-DEC205. In some embodiments, the scFv with specificity for murine CD205 comprises or consists of SEQ ID NO: 49.

In other embodiments, the targeting unit comprises or consists of an amino acid sequence having at least 80% sequence identity to that of human CTLA4, such as an amino acid sequence having at least 80% sequence identity to SEQ ID NO: 217.

In yet other embodiments, the targeting unit comprises or consists of an amino acid sequence having at least 85% sequence identity to the amino acid sequence of human CTLA4, such as an amino acid sequence having at least 85% sequence identity to SEQ ID NO: 217, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human CTLA4, such as the amino acid sequence of SEQ ID NO: 217, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human CTLA4, or a nucleotide sequence encoding human CTLA4.

In other embodiments, the targeting unit comprises or consists of or a nucleotide sequence encoding human CTLA4, such as the nucleotide sequence of SEQ ID NO: 218.

In yet other embodiments, the targeting unit comprises or consists of an amino acid sequence having at least 80% sequence identity to that of murine CTLA4, such as murine CTLA4 as set forth in SEQ ID NO: 175.

In other embodiments, the targeting unit comprises or consists of an amino acid sequence having at least 80% sequence identity to that of human PD-1, such as an amino acid sequence having at least 80% sequence identity to SEQ ID NO: 219.

In yet other embodiments, the targeting unit comprises or consists of an amino acid sequence having at least 85% sequence identity to the amino acid sequence of human PD-1, such as an amino acid sequence having at least 85% sequence identity to SEQ ID NO: 219, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human PD-1, such as the amino acid sequence of SEQ ID NO: 219, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human PD-1, or a nucleotide sequence encoding human PD-1.

In other embodiments, the targeting unit comprises or consists of or a nucleotide sequence encoding human PD-1, such as the nucleotide sequence of SEQ ID NO: 220.

In yet other embodiments, the targeting unit comprises or consists of an amino acid sequence having at least 80% sequence identity to that of murine PD-1, such as murine PD-1 as set forth in SEQ ID NO: 179.

In yet other embodiments, the targeting unit comprises or consists of an amino acid sequence having at least 80% sequence identity to that of human IL-10, such as an amino acid sequence having at least 80% sequence identity to SEQ ID NO: 211.

In yet other embodiments, the targeting unit comprises or consists of an amino acid sequence having at least 85% sequence identity to the amino acid sequence of human IL-10, such as an amino acid sequence having at least 85% sequence identity to SEQ ID NO: 211, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human IL-10, such as the amino acid sequence of SEQ ID NO: 211, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, the targeting unit comprises or consists of an amino acid sequence of human IL-10, or a nucleotide sequence encoding human IL-10.

In other embodiments, the targeting unit comprises or consists of or a nucleotide sequence encoding human IL-10, such as the nucleotide sequence of SEQ ID NO: 212.

### Antigenic Unit

The antigenic unit of the tolerance-inducing construct of the disclosure comprises one or more T cell epitopes of a self-antigen, an allergen, an alloantigen or a xenoantigen.

T cell epitopes suitable for inclusion into the antigenic unit may be known in the art, i.e. have been studied, proposed and/or verified to be involved and of relevance for a certain immune disease and published in the literature.

In some embodiments, the antigenic unit comprises one or more T cell epitopes of a self-antigen, i.e. one T cell epitope of a self-antigen or more than one T cell epitope of a self-antigen, i.e. multiple T cell epitopes of a self-antigen. In some embodiments, the multiple T cell epitopes are of the same self-antigen, i.e. comprised in the same self-antigen. In other embodiments, the multiple T cell epitopes are of multiple different self-antigens, i.e. comprised in different self-antigens.

In some embodiments, the antigenic unit comprises one or more T cell epitopes of a self-antigen, such as T reg epitopes or inhibitory neoantigens.

In some embodiments, where the antigenic unit comprises more than one T cell epitope, the antigenic unit comprises one or more linkers separating the T cell epitopes. In some embodiments, the antigenic unit comprises multiple T cell epitopes of a self-antigen, an allergen, an alloantigen or a xenoantigen, wherein the T cell epitopes are preferably separated by a linkers. In yet other embodiments, the antigenic unit comprises multiple T cell epitopes of a self-antigen, an allergen, an alloantigen or a xenoantigen wherein each T cell epitope is separated from other T cell epitopes by linkers. An alternative way to describe the separation of each T cell epitope of a self-antigen, an allergen, an alloantigen or a xenoantigen from other T cell epitopes by linkers is that all but the terminal T cell epitopes, i.e. the T cell epitope at the N-terminal start of the polypeptide or the C-terminal end of the polypeptide (i.e. located at the end of the antigenic unit that is not connected to the dimerization unit), are arranged in subunits, wherein each subunit comprises or consists of a T cell epitope and a linker as described herein.

Hence, an antigenic unit comprising n antigens comprises n−1 subunits, wherein each subunit comprises a T cell epitope of a self-antigen, an allergen, an alloantigen or a xenoantigen, and a linker, and further comprises a terminal T cell epitope. In some embodiments, n is an integer of from 1 to 50, e.g. 3 to 50 or 15 to 40 or 10 to 30 or 10 to 25 or 10 to 20 or 15 to 30 or 15 to 25 or 15 to 20.

Linkers in the antigenic unit separate antigens comprised therein, e.g. epitopes. As described above, all T cell epitopes of a self-antigen, an allergen, an alloantigen or a xenoantigen, may be separated from each other by linkers and arranged in subunits.

In some embodiments, the linker is designed to be non-immunogenic. It may be a rigid linker, meaning that it does not allow the two amino acid sequences that it connects to substantially move freely relative to each other. Alternatively, it may be a flexible linker, i.e. a linker that allows the two amino acid sequences that it connects to substantially move freely relative to each other.

Both types of linkers are useful. In one embodiment, the T cell epitope linker is a flexible linker, which allows for presenting the T cell epitopes in an optimal manner to the T cells, even if the antigenic unit comprises a large number of T cell epitopes.

Due to the separation of the T cell epitopes by the linkers, each T cell epitope of a self-antigen, an allergen, an alloantigen or a xenoantigen is presented in an optimal way to the immune system.

By way of example, myelin basic protein (MBP), proteolipid protein (PLP), myelin-associated glycoprotein (MAG), myelin oligodendrocyte glycoprotein (MOG) and myelin-associated basic oligodendrocytic protein (MOBP) have all been studied and proposed as self-antigens involved in multiple sclerosis (MS) and the antigenic unit may comprise e.g. one or more T cell epitopes of MBP, i.e. one T cell epitope of MBP or multiple T cell epitopes of MBP. Further, the antigenic unit may comprise multiple T cell epitopes of e.g. MOG and PLP, e.g. one or multiple T cell epitopes of MOG and one or multiple T cell epitopes of PLP.

In some embodiments, the antigenic unit may comprise one or multiple T cell epitopes of MOG, such as one or multiple T cell epitopes of MOG comprising or consisting of a sequence selected from the group consisting of SEQ ID NO: 180-182.

In other embodiments, the antigenic unit comprises one or more T cell epitopes of an allergen, i.e. one T cell epitope of an allergen or more than one T cell epitope of an allergen, i.e. multiple T cell epitopes of an allergen. In some embodiments, the multiple T cell epitopes are of the same allergen, i.e. comprised in the same allergen. In other embodiments, the multiple T cell epitopes are of multiple different allergens, i.e. comprised in different allergens.

By way of example, Fel d1, Fel d4 and Fel d7 are three of the most prominent cat allergens, accounting for the majority of human cat allergies and the antigenic unit may comprise e.g. one or more T cell epitopes of Fel d1, i.e. one T cell epitope of Fel d1 or multiple T cell epitopes of Fel d1. Further, the antigenic unit may comprise multiple T cell epitopes of e.g. Fel d4 and Fel d7, e.g. one or multiple T cell epitopes of Fel d4 and one or multiple T cell epitopes of Fel d7.

In some embodiments, the antigenic unit may comprise one or multiple T cell epitopes of Met e 1, such as one or multiple T cell epitopes comprised in SEQ ID NO: 184. In some embodiments, the antigenic unit may comprise one or multiple T cell epitopes of Met e 1, such as Met e 1(16-35), Met e 1(46-65), Met e 1(76-95), Met e 1(136-155), Met e 1(210-230) and/or Met e 1(241-260). In some embodiments, the antigenic unit may comprise one or multiple T cell epitopes of Met e 1, such as one or multiple T cell epitopes comprising or consisting of a sequence selected from any of SEQ ID NO: 185-190.

In other embodiments, the antigenic unit comprises one or more T cell epitopes of an alloantigen/xenoantigen, i.e. one T cell epitope of an alloantigen/xenoantigen or more than one T cell epitope of an alloantigen/xenoantigen, i.e. multiple T cell epitopes of an alloantigen/xenoantigen. In some embodiments, the multiple T cell epitopes are of the same alloantigen/xenoantigen, i.e. comprised in the same alloantigen/xenoantigen. In other embodiments, the multiple T cell epitopes are of multiple different alloantigen/xenoantigens, i.e. comprised in different alloantigens/xenoantigens.

In some embodiments, the antigenic unit includes one T cell epitope. In other embodiments, the antigenic unit includes more than one T cell epitope, i.e. multiple T cell epitopes.

The tolerance-inducing construct of the disclosure may be an individualized treatment, i.e. designed for a particular subject/one patient. In other embodiments, the tolerance-inducing construct of the disclosure is for general use in a patient population or patients, i.e. an off-the-shelf treatment.

### Individualized Tolerance-Inducing Constructs

For individualized tolerance-inducing constructs, T cell epitopes are selected for inclusion into the antigenic unit, which T cell epitopes are optimized for the patient who will receive treatment with the construct. This will increase the therapeutic effect compared to an off-the-shelf treatment comprising the tolerance-inducing construct.

The antigenic unit of an individualized tolerance-inducing construct may be designed as follows, as exemplified for a patient suffering from MS:
1) The patient's HLA class I and/or HLA class II alleles are determined
2) T cell epitopes are identified comprised in one or more self-antigens (e.g. self-antigens which have been studied, proposed and/or verified as self-antigens involved in MS)
3) T cell epitopes are selected based on predicted binding to the patient's HLA class I and/or class II alleles
4) One or more tolerance-inducing test constructs are designed and produced, and the T cell epitopes are optionally arranged in the antigenic unit of the constructs as described in this application

The T cell epitopes are selected in the method described above based on their predicted ability to bind to the patient's HLA class I/II alleles, i.e. selected in silico using predictive HLA-binding algorithms. After having identified relevant epitopes, the epitopes are ranked according to their ability to bind to the patient's HLA class I/II alleles and the epitopes that are predicted to bind best are selected to be included in the antigenic unit of the test constructs.

Any suitable HLA-binding algorithm may be used, such as one of the following: Available software analysis of peptide-MHC binding (IEDB, NetMHCpan and NetMHCIIpan) may be downloaded or used online from the following websites: www.iedb.org/services.healthtech.dtu.dk/service.php?NetMHCpan-4.0 services.healthtech.dtu.dk/service.php?NetMHCIIpan-3.2

### Off-the-Shelf Tolerance Inducing Constructs

The antigenic unit of an off-the-shelf tolerance inducing construct preferably includes hotspots of minimal T cell epitopes, i.e. one or more regions of an antigen that contain multiple minimal T cell epitopes (e.g. having a length of from 7-15 amino acids) that are predicted to be presented by different HLA alleles to cover a broad range of subjects, e.g. an ethnic population or even a world population or global population.

By including such hotspots, chances are maximized that the construct will induce tolerance in a broad range of subjects.

### Further Description of the Antigenic Unit

The T cell epitope comprised in the antigenic unit of the construct of the disclosure has a length of from 7 to about 200 amino acids, with the longer T cell epitopes possibly including hotspots of minimal epitopes.

In some embodiments, the antigenic unit comprises T cell epitopes with a length from 7 to 150 amino acids, preferably from 7 to 100 amino acids, e.g. from 9 to 100 amino acids or from 15 to 100 amino acids or from 9 to 60 amino acids or from 9 to 30 amino acids or from 15 to 60 or from 15 to 30 or from 20 to 75 amino acids or from 25 to 50 amino acids, such as 25, 26, 27, 28, 29, 30, 31, 32, 33, 34, 35, 36, 37, 38, 39, 40, 41, 42, 43, 44, 45, 46, 47, 48, 49, or 50 amino acids.

T cell epitopes having a length of about 60 to 200 amino acids may be split into shorter sequences and included into the antigenic unit separated by the linkers which are described herein. By way of example, a T cell epitope having a length of 150 amino acids may be split into 3 sequences of 50 amino acids each, and included into the antigenic unit, with a linker separating the 3 sequences from each other.

In some embodiments, the length of one T cell epitope is such that the protein does not fold correctly. For example, Fel d 1, the most prominent cat allergen, is a protein formed by two heterodimers, with each dimer being composed of two chains, chain 1 comprising 70 amino acid residues and chain 2, comprising 90 or 92 residues.

Including long T cell epitopes of both chains into the antigenic unit may result in the proteins folding correctly and, if more than one IgE on the subject's mast cells and basophiles binds the antigenic unit of the construct, might elicit an allergic reaction.

If a longer T cell epitope is included in the antigenic unit, protein folding may be tested in vitro by e.g. ELISA, using an antibody against the protein (e.g. cat allergen) and determining whether the antibody binds to the T cell epitope.

In some embodiments, the T cell epitope has a length suitable for presentation by MHC (major histocompatibility complex). There are two primary classes of MHC molecules, MHC class I and MHC II. The terms MHC class I and MHC class II are interchangeably used herein with HLA class I and HLA class II. HLA (human leukocyte antigen) is a major histocompatibility complex in humans. Thus, in one, the antigenic unit comprises T cell epitopes having a length suitable for specific presentation on MHC class I or MHC class II. In some embodiments, the T cell epitope has a length of from 7 to 11 amino acids for MHC class I presentation. In other embodiments, the T cell epitope sequence has a length of from 9 to 60 amino acids, such as from 9 to 30 amino acids, such as 15 to 60 amino acids, such as 15 to 30 amino acids for MHC class II presentation. In other embodiments, the T cell epitope has a length of 15 amino acids for MHC class II presentation.

The number of T cell epitopes in the antigenic unit may vary, and depends on the length and number of other elements included in the antigenic unit, e.g. T cell epitope linkers as described in this application.

In some embodiments, the antigenic unit comprises up to 3500 amino acids, such as from 60 to 3500 amino acids, e.g. from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.

In some embodiments, the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes. In other embodiments, the antigenic unit comprises 1 to 3 T cell epitopes, such as 1, 2, 3, or 1 to 5 T cell epitopes, such as 1, 2, 3, 4, 5, or 3 to 6 T cell epitopes, such as 3, 4, 5, 6, or 5 to 15 T cell epitopes, such as 5, 6, 7, 8, 9, 10, 11, 12, 13, 14, or 15 T cell epitopes, or 7 to 17 T cell epitopes, such as 7, 8, 9, 10, 11, 12, 13, 14, 15, 16, or 17 T cell epitopes, or 9 to 19 T cell epitopes, such as 9, 10, 11, 12, 13, 14, 15, 16, 17, 18, or 19 T cell epitopes.

In some embodiments, the T cell epitopes are randomly arranged in the antigenic unit. In other embodiments, one or more of the following methods for arranging them in the antigenic unit may be used.

In some embodiments, the T cell epitopes are arranged in the order of more antigenic to less antigenic in the direction from the multimerization unit, such as the dimerization unit, to the end of the antigenic unit. Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the multimerization unit, such as the dimerization unit, or the end of the antigenic unit.

In some embodiments, the T cell epitopes are arranged in the order of more antigenic to less antigenic in the direction from the multimerization unit to the end of the antigenic unit. Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the multimerization unit or the end of the antigenic unit.

In some embodiments, the T cell epitopes are arranged in the order of more antigenic to less antigenic in the direction from dimerization unit to the end of the antigenic unit (see FIG. 1). Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the dimerization unit or the end of the antigenic unit.

Since a true positioning in the middle of the antigenic unit is only possible if the antigenic unit comprises an odd number of T cell epitopes, the term “substantially” in this context refers to antigenic units comprising an even number of T cell epitopes, wherein the most hydrophobic T cell epitopes are positioned as close to the middle as possible.

By way of example, an antigenic unit comprises 5 T cell epitopes, which are arranged as follows: 1-2-3*-4-5; with 1, 2, 3*, 4 and 5 each being a different T cell epitope and -being a T cell epitope linker and * indicating the most hydrophobic T cell epitope, which is positioned in the middle of the antigenic unit.

In another example, a antigenic unit comprises 6 T cell epitopes, which are arranged as follows: 1-2-3*-4-5-6 or, alternatively, as follows: 1-2-4-3*-5-6; with 1, 2, 3*, 4, 5 and 6 each being a T cell epitope and—being a T cell epitope linker and * indicating the most hydrophobic T cell epitope, which is positioned substantially in the middle of the antigenic unit.

Alternatively, the T cell epitopes may be arranged alternating between a hydrophilic and a hydrophobic T cell epitope. Optionally, GC rich T cell epitopes are arranged in such a way, that GC clusters are avoided. In preferred embodiments, GC rich T cell epitopes are arranged such that there is at least one non-GC rich T cell epitope between them. In some embodiments, GC rich sequences encoding T cell epitopes are arranged such that there is at least one non-GC rich T cell sequence between them. GC rich sequences are sequences with a GC content of 60% or more, such as 65% or more, such as 70% or more, such as 75% or more, such as 80% or more.

If the antigenic unit comprises multiple T cell epitopes, the epitopes are preferably separated by T cell epitope linkers. This ensures that each T cell epitope is presented in an optimal way to the immune system. If the antigenic unit comprises n T cell epitopes, it preferably comprises n−1 T cell epitope linkers, separating each T cell epitope from one or two other T cell epitopes.

The T cell epitope linker is designed to be non-immunogenic and is preferably also a flexible linker, which allows for presenting the T cell epitope in an optimal manner to the immune system, even if the antigenic unit comprises a large number of T cell epitopes.

Preferably, the T cell epitope linker is a peptide consisting of from 4 to 20 amino acids, e.g. from 5 to 20 amino acids or 5 to 15 amino acids or 8 to 20 amino acids or 8 to 15 amino acids, such as 8, 9, 10, 11, 12, 13, 14, or 15 amino acids 10 to 15 amino acids or 8 to 12 amino acids, such as 8, 9, 10, 11, or 12 amino acids. In particular preferred embodiments, the T cell epitope linker consists of 10 amino acids.

All T cell epitope linkers comprised in the antigenic unit are preferably identical. If, however, one or more of the T cell epitopes comprises a sequence similar to that of the linker, it may be an advantage to substitute the neighbouring T cell epitope linker with a linker of a different sequence. Also, if a T cell epitope/linker junction is predicted to constitute an epitope, then it is preferred to use a T cell epitope linker of a different sequence.

In one embodiment, the T cell epitope linker is designed to be non-immunogenic. It may be a rigid linker, meaning that that it does not allow the two amino acid sequences that it connects to substantially move freely relative to each other. Alternatively, it may be a flexible linker, i.e. a linker that allows the two amino acid sequences that it connects to substantially move freely relative to each other.

Both types of linkers are useful. In one embodiment, the T cell epitope linker is a flexible linker, which allows for presenting the T cell epitopes in an optimal manner to the T cells, even if the antigenic unit comprises a large number of T cell epitopes.

Preferably, the T cell epitope linker is a serine (S) and/or glycine (G) rich linker, i.e. a linker comprising several serine and/or several glycine residues. Preferred examples are GGGGSGGGSS (SEQ ID NO: 51), GGGSG (SEQ ID NO: 52), GGGGS (SEQ ID NO: 53), SGSSGS (SEQ ID NO: 54), GGSGG (SEQ ID NO: 55) or multiple variants thereof such as GGGGSGGGGS (SEQ ID NO: 56), (GGGGS)m (SEQ ID NOs: 53, and 56-59), (GGGSS)m (SEQ ID NOs: 60-64), (GGGSG)m (SEQ ID NOs: 52 and 65-68), or (SGSSGS)m (SEQ ID NOs: 54 and 69-72), where m is an integer from 1 to 5, e.g., 1, 2, 3, 4, or 5, In preferred embodiments, m is 2. In other preferred embodiments, the serine and/or glycine rich linker further comprises at least one leucine (L) residue, such as at least 1 or at least 2 or at least 3 leucine residues, e.g. 1, 2, 3 or 4 leucine residues.

In some embodiments, the T cell epitope linker comprises or consists of LGGGS (SEQ ID NO: 73), GLGGS (SEQ ID NO: 74), GGLGS (SEQ ID NO: 75), GGGLS (SEQ ID NO: 76) or GGGGL (SEQ ID NO: 77). In other embodiments, the T cell epitope linker comprises or consists of LGGSG (SEQ ID NO: 78), GLGSG (SEQ ID NO: 79), GGLSG (SEQ ID NO: 80), GGGLG (SEQ ID NO: 81) or GGGSL (SEQ ID NO: 82). In yet other embodiments, the T cell epitope linker comprises or consists of LGGSS (SEQ ID NO: 83), GLGSS (SEQ ID NO: 84), or GGLSS (SEQ ID NO: 85).

In yet other embodiments, the T cell epitope linker comprises or consists of LGLGS (SEQ ID NO: 86), GLGLS (SEQ ID NO: 87), GLLGS (SEQ ID NO: 88), LGGLS (SEQ ID NO: 89), GLGGL (SEQ ID NO: 90) or (GLGGL)m (SEQ ID NOs: 90-94). In yet other embodiments, the T cell epitope linker comprises or consists of LGLSG (SEQ ID NO: 95), GLLSG (SEQ ID NO: 96), GGLSL (SEQ ID NO: 97), GGLLG (SEQ ID NO: 98) or GLGSL (SEQ ID NO: 99). In yet other embodiments, the T cell epitope linker comprises or consists of LGLSS (SEQ ID NO: 100), or GGLLS (SEQ ID NO: 101).

In other embodiments, the T cell epitope linker is serine-glycine linker that has a length of 10 amino acids and comprises 1 or 2 leucine residues.

In some embodiments, the T cell epitope linker comprises or consists of LGGGSGGGGS (SEQ ID NO: 102), GLGGSGGGGS (SEQ ID NO: 103), GGLGSGGGGS (SEQ ID NO: 104), GGGLSGGGGS (SEQ ID NO: 105) or GGGGLGGGGS (SEQ ID NO: 106). In other embodiments, the T cell epitope linker comprises or consists of LGGSGGGGSG (SEQ ID NO: 107), GLGSGGGGSG (SEQ ID NO: 108), GGLSGGGGSG (SEQ ID NO: 109), GGGLGGGGSG (SEQ ID NO: 110) or GGGSLGGGSG (SEQ ID NO: 111). In yet other embodiments, the T cell epitope linker comprises or consists of LGGSSGGGSS (SEQ ID NO: 112), GLGSSGGGSS (SEQ ID NO: 113), GGLSSGGGSS (SEQ ID NO: 114), GGGLSGGGSS (SEQ ID NO: 115) or GGGSLGGGSS (SEQ ID NO: 116).

In further embodiments, the T cell epitope linker comprises or consists of LGGGSLGGGS (SEQ ID NO: 117), GLGGSGLGGS (SEQ ID NO: 118), GGLGSGGLGS (SEQ ID NO: 119), GGGLSGGGLS (SEQ ID NO: 120) or GGGGLGGGGL (SEQ ID NO: 121). In other embodiments, the T cell epitope linker comprises or consists of LGGSGLGGSG (SEQ ID NO: 122), GLGSGGLGSG (SEQ ID NO: 123), GGLSGGGLSG (SEQ ID NO: 124), GGGLGGGGLG (SEQ ID NO: 125) or GGGSLGGGSL (SEQ ID NO: 126). In yet other embodiments, the T cell epitope linker comprises or consists of LGGSSLGGSS (SEQ ID NO: 127), GLGSSGLGSS (SEQ ID NO: 128), or GGLSSGGLSS (SEQ ID NO: 129).

In yet other embodiments, the T cell epitope linker comprises or consists of GSGGGA (SEQ ID NO: 130), GSGGGAGSGGGA (SEQ ID NO: 131), GSGGGAGSGGGAGSGGGA (SEQ ID NO: 132), GSGGGAGSGGGAGSGGGAGSGGGA (SEQ ID NO: 133) or GENLYFQSGG (SEQ ID NO: 134). In yet other embodiments, the flexible unit comprises or consists of SGGGSSGGGS (SEQ ID NO: 135), GGGGSGGGGS (SEQ ID NO: 56), SSGGGSSGGG (SEQ ID NO: 136), GGSGGGGSGG (SEQ ID NO: 137), GSGSGSGSGS (SEQ ID NO: 138), GGGSSGGGSG (SEQ ID NO: 139), GGGSSS (SEQ ID NO: 140), GGGSSGGGSSGGGSS (SEQ ID NO: 62) or GLGGLAAA (SEQ ID NO: 141).

In other embodiments, the T cell epitope linker is a rigid linker. Such rigid linkers may be useful to efficiently separate (larger) antigens and prevent their interferences with each other. In one embodiment, the T cell epitope linker comprises or consists of KPEPKPAPAPKP (SEQ ID NO: 142), AEAAAKEAAAKA (SEQ ID NO: 143), (EAAAK)m (SEQ ID NOs: 144-148), PSRLEEELRRRLTEP (SEQ ID NO: 149) or SACYCELS (SEQ ID NO: 150).

In other embodiments, the T cell epitope linker comprises or consists of the sequence TQKSLSLSPGKGLGGL (SEQ ID NO: 151). In other embodiments, the T cell epitope linker comprises or consists of the sequence SLSLSPGKGLGGL (SEQ ID NO: 152). In other embodiments, the T cell epitope linker comprises or consists of AAY or GPGPG (SEQ ID NO: 153).

In yet other embodiments, the T cell epitope linker is a GSAT linker, i.e. a linker comprising one or more glycine, serine, alanine and threonine residues, e.g. a linker comprising or consisting of the sequence GGSAGGSGSGSSGGSSGASGTGTAGGTGSGSGTGSG (SEQ ID NO: 154) or a SEG linker, i.e. a linker comprising one or more serine, glutamic acid and glycine residues, e.g. a linker comprising or consisting of the sequence GGSGGGSEGGGSEGGGSEGGGSEGGGSEGGGSGGGS (SEQ ID NO: 155) or ELKTPLGDTTHT (SEQ ID NO: 156).

In other embodiments, the T cell epitope linker is a cleavable linker, e.g. a linker which includes one or more recognition sites for endopeptidases, e.g. endopeptidases such as furin, caspases, cathepsins and the like. Cleavable linkers may be introduced to release free functional protein domains (e.g. encoded by larger antigens), which may overcome steric hindrance between such domains or other drawbacks due to interference of such domains, like decreased bioactivity, altered biodistribution.

Examples of T cell epitope linkers are disclosed in paragraphs [0098]-[0099] and in the recited sequences of WO 2020/176797A1 (in particular SEQ ID NOs: 37 to 65 and SEQ ID NOs: 67 to 76), which is incorporated herein by reference and in paragraphs [0135] to [0139] of US 2019/0022202A1, which is incorporated herein by reference.

### Allergens

The tolerance-inducing construct as described herein is useful for inducing tolerance to a range of different protein allergens, e.g. allergens that can be encoded by a nucleic acid sequence comprised in the polynucleotide of the constructs of the disclosure, including protein allergens that undergo post-translational modifications.

In some embodiments, the allergen is a food allergen. In some embodiments, the allergen is a shellfish allergen. In some embodiments, the allergen is tropomyosin, in other embodiments the allergen is Arginin kinase, myosin light chain, sarcoplasmic calcium binding protein, troponin C or Triose-phosphate isomerase or actin. In some embodiments, the allergen is Pan b 1. In some embodiments the antigen unit is Pan b 1 T cell epitope (251-270).

In some embodiments, the allergen is a cow's milk allergen. In some embodiments, the cow's milk allergen is Bos d 4, Bos d 5, Bos d 6, Bos d 7, Bos d 8, Bos d 9, Bos d 10, Bos d 11 or Bos d 12.

In some embodiments, the allergen is an egg allergen. In some embodiments, the egg allergen is ovomucoid, in other embodiments the egg allergen is ovalbumin, ovotransferin, conalbumin, Gal 3 3, egg lyaozyme or ovomucin.

One T cell epitope that is known in the art and has been studied in the context of egg allergy is OVA (257-264), with amino acid sequence SIINFEKL (SEQ ID NO: 45).

In some embodiments, the antigenic unit of the construct according to disclosure comprises the T cell epitope OVA (257-264). A pharmaceutical composition comprising said T cell epitope may be used in the treatment of egg allergy.

In some embodiments, the allergen is a fish allergen. In some embodiments, the fish allergen is a parvalbumin. In other embodiments the fish allergen is enolase, aldolase or vitellogenin. In some embodiments, the allergen is a fruit allergen. In some embodiments, the fruit allergen is pathogenesis related protein 10, profilin, nsLTP, thaumatin-like protein, gibberellin regulated protein, isoflavone reductase related protein, class 1 chitinase, beta 1,3 glucanase, germin like protein, alkaline serine protease, pathogenesis-related protein 1, actinidin, phytocyctatin, kiwellin, major latex protein, cupin or 2S albumin. In some embodiments, the allergen is a vegetable allergen. In some embodiments, the vegetable allergen is pathgenesis related protein 10, profilin, nsLTP type 1, nsLTP type protein 2, osmotin-like protein, isoflavone reductase-like protein, beta-fructofuranosidase, PR protein TSI-1, cyclophilin or FAD containing oxidase.

In some embodiments, the allergen is a wheat allergen. In some embodiments, the wheat allergen is Tri a 12, Tri a 14, Tri a 15, Tri a 18, Tri a 19, Tri a 20, Tri a 21, Tri a 25, Tri a 26, Tri a 27, Tri a 28, Tri a 29, Tri a 30, Tri a 31, Tri a 32, Tri a 33, Tri a 34, Tri a 35, Tri a 36, Tri a 37 or Tri a 38. In some embodiments, the allergen is a soy allergen. In some embodiments, the soy allergen is Gly m 1, Gly m 2, Gly m 3, Gly m 4, Gly m 5, Gly m 6, Gly m 7 or Gly m 8. In other embodiments the soy allergen is Gly m agglutinin, Gly m Bd28K, Gly m 30 kD, Gly m CPI or Gly m TI. In some embodiments, the allergen is a peanut allergen. In some embodiments, the peanut allergen is Ara h 1, Ara h 2, Ara h 3, Ara h 5, Ara h 6, Ara h 7, Ara h 8, Ara h 9, Ara h 10, Ara h 11, Ara h 12, Ara h 13, Ara h 14, Ara h 15, Ara h 16, or Ara h 17. In some embodiments, the allergen is a tree nut or seed allergen. In some embodiments, the allergen is 11 S globulin, 7S globulin, 2S globulin, PR10, PR-14 nsLTP, Oleosin or profilin.

In other embodiments the food allergen is buckwheat, celery, a color additive, garlic, gluten, oats, legumes, maize, mustard, poultry, meat, rice, sesame, or derived from buckwheat, celery, a color additive, garlic, gluten, oats, legumes, maize, mustard, poultry, meat, rice, sesame.

In some embodiments, the allergen is a bee venom allergen. In some embodiments, the bee venom allergen is Phospholipase A2, Hyaluronidase, acid phosphatase, melittin, allergen C/DPP, CRP/lcarapin or vitellogenin. In some embodiments, the allergen is a vespid allergen. In some embodiments, the vespid allergen is Phospholipase A1, hyaluronidase, protease, antigen 5, DPP IV or vitellogenin.

In some embodiments, the allergen is a latex allergen. In some embodiments, the latex allergen is Hev b 1, Hev b 2, Hev b 3, Hev b 4, Hev b 5, Hev b 6, Hev b 7, Hev b 8, Hev b9, Hevb 10, Hevb 11, Hevb 12, Hevb 13, Hevb 14, Hevb 15.

In some embodiments, the allergen is a dust mite allergen. In some embodiments the allergen is a house dust mite allergen. In some embodiments, the allergen is a storage dust allergen. In some embodiments, the house dust mite allergen is Der p 1, Der p 2, Der p 3, Der p 4, Der p 5, Der p 7, Der p 8, Der p 10, Der p 11, Der p 21, or Der p 23.

In some embodiments the antigen unit is the Der p 1 T cell epitope (111-139). In some embodiments, the house dust mite allergen is Der f 1, Der f 2, Der f 3, Der f 7, Der f 8 or Der f 10. In some embodiments, the house dust mite allergen is Blot t 1, Blot t 2, Blot t 3, Blot t 4, Blot t 5, Blot t 8, Blot t 10, Blot t 12 or Blot t 21.

In some embodiments, the allergen is a cockroach allergen. In some embodiments, the cockroach allergen is Bla g 1, Bla g 2, Bla g 3, Bla g 4, Bla g 5, Bla g 6, Bla g 7, Bla g 8 or Bla g 11. In some embodiments, the cockroach allergen is Per a 1, Per a 2, Per a 3, Per a 6, Per a 7, Per a 9 or Per a 10.

In some embodiments, the allergen is a mold allergen. In some embodiments, the mold allergen is an Aspergillus fumigatus allergen. In some embodiments, the Aspergillus fumigatus allergen is Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 14, Asp f 15, Asp f 16, Asp f 17, Asp f 18, Asp f22, Asp f23, Asp f27, Asp f28, Asp f29 or Asp f 34.

In some embodiments, the allergen is a fungal allergen. In some embodiments, the fungal allergen is a Malassezia allergen. In some embodiments, the Malassezia allergen is Mala f 1, Mala f 2, Mala f 3, Mala f 4, Mala f 5, Mala f 6, Mala f 7, Mala f 8, Mala f 9, Mala f 10, Mala f 11, Mala f 12 or Mala f 13 or MGL_1204.

In some embodiments, the allergen is furry animal allergen. In some embodiments, the allergen is a dog allergen. In some embodiments, the dog allergen is Can f 1, Can f 2, Can f 3, Can f 4, Can f 5, or Can f 6. In some embodiments, the allergen is a horse allergen. In some embodiments, the horse allergen is Ecu c 1, Ecu c 2, Ecu c 3 or Ecu c 4. In some embodiments, the allergen is a cat allergen. In some embodiments, the cat allergen is Fel d 1, Fel d 2, Fel d 3, Fel d 4, Fel d 5, Fel d 6, Fel d 7, or Fel d 8. In some embodiments, the allergen is a laboratory animal allergen. In some embodiments, the allergen is Lipocalin, urinary prealbumin, secretoglobulin or serum albumin.

In some embodiments, the allergen is a pollen allergen. In some embodiments, the allergen is a grass pollen allergen. In some embodiments, the grass pollen allergen is a timothy grass, orchard grass, Kentucky bluegrass, perennial rye, sweet vernal grass, bahia grass, johnson grass or Bermuda grass allergen. In some embodiments the grass pollen allergen is Phl p 1, Phl p 2, Phl p 3, Phl p 4, Phl p 5, Phl p 6, Phl p 7, Phl p 11, Phl p 12 or Phl p 13.

In some embodiments, the allergen is a tree pollen allergen. In some embodiments, the tree pollen allergen is an alder, birch, hornbeam, hazel, European hophornbeam, chestnut, European beech, white oak, ash, privet, olive, lilac, cypress or cedar pollen allergen. In some embodiments, the tree pollen allergen is Aln g 1 or Aln g 4, Bet v 1, Bet v 2, Bet v 3, Bet v 4, Bet v 6 or Bet v 7, Car b 1, Cora 1, Cor a 2, Cor a 6, Cora 8, Cor a 9, Cor a 10, Cor a 11, Cor a 12, Cor a 13, Cor a 14, Ost c 1, Cas 1, Cas 5, Cas 8, or Cas 9, Fag s 1, Que a 1, Fra e 1, Lig v 1, Ole e 1, Ole e 2, 3 Ole e, 4, Ole e 5, Ole e 6, Ole e 7, Ole e 8, Ole e 9, Ole e 10, Ole e 11, or Ole e 12, Syr v 1, Cha o 1, Chao 2, Cry j 1, Cry j 2, Cup s 1, Cup s 3, Jun a 1, Jun a 2, Jun a 3, Jun o 4, Jun v 1, Jun v 3, Pla a 1, Pla a 2 or Pla a 3 or Pla or 1, Pla or 2 or Pla or 3. In some embodiments, the antigen unit will be the Bet v 1 T cell epitope (139-152).

In some embodiments, the allergen is a weed pollen allergen. In some embodiments the weed allergen is a ragweed, mugwort, sunflower, feverfew, pellitory, English plantain, annual mercury, goosefoot, Russian thistle or amaranth pollen allergen. In some embodiments the ragweed pollen allergen is Amb a 1, Amb a 4, Amb a 6, Amb a 8, Amb a 9, Amb a 10, or Amb a 11. In some embodiments the mugwort pollen allergen is Art v 1, Art v 3, Art v 4, Art v 5, or Art v 6. In some embodiments, the sunflower pollen allergen is Hel a 1 or Hel a 2. In some embodiments, the pellitory pollen allergen is Par j 1, Par j 2, Par j 3 or Par j 4. In some embodiments, the English plantain pollen allergen is Pla I 1. In some embodiments, the annual mercury pollen allergen is Mer a 1. In some embodiments, the goosefoot pollen allergen is Che a 1, Che a 2 or Che a 3. In some embodiments, the Russian thistle pollen allergen is Sal k 1, Sal k 4 or Sal k 5. In some embodiments, the Amaranth pollen allergen is Ama r 2.

In yet other embodiments the allergen is selected form environmental allergens such as insects, cockroaches, house dust mites or mold.

In some embodiments, the allergic disease is allergic rhinitis, asthma, atopic dermatitis, allergic gastroenteropathy, contact dermatitis, drug allergy or combinations thereof.

Allergy to drugs affect more than 7% of the general population. The constructs of the disclosure induce tolerance towards immunogenic epitopes present in such a drug and thus will allow affected patients to continue treatment with the drug and receive the benefits from the drug treatment.

Thus, in some embodiments, the allergen is comprised in a drug with unwanted immunogenicity. In some embodiments, the allergen is Factor VIII. In some embodiments, the allergen is insulin. In some embodiments, the allergen is one or more monoclonal antibodies used for therapy.

### Self-Antigens

In other embodiments, the present tolerance-inducing construct contains T cell epitopes comprised in a self-allergen that is involved in an autoimmune disease. This allows for the antigen-specific down-regulation of the part of the immune system responsible for the autoimmune disease without inhibiting the immune system in general.

In some embodiments, the autoimmune disease is multiple sclerosis (MS). In some embodiments, the self-antigen is myelin oligodendrocyte glycoprotein (MOG). In other embodiments the self-antigen is MAG, MOBP, CNPase, S100beta or transaldolase. In some embodiments, the self-antigen is myelin basic protein (MBP). In some embodiments, the self-antigen is myelin proteolipid protein (PLP).

In the examples we provide constructs for multiple sclerosis including either a short (35-55 amino acids) or a longer (27-63 amino acids) T cell epitope from myelin oligodendrocyte glycoprotein (MOG). MOG is a member of the immunoglobulin superfamily and is expressed exclusively in the central nervous system. MOG (35-55) can induce autoantibody production and relapsing-remitting neurological disease, causing extensive plaque-like demyelination. Autoantibody response to MOG (35-55) has been observed in MS patients and MOG (35-55)-induced experimental autoimmune encephalomyelitis (EAE) in C57/BL6 mice and Lewis rats.

Other MS-relevant T cell epitopes that are known in the art and have been studied include the following:

| T cell epitope | Sequence |
|---|---|
| PLP (139-151)* | HCLGKWLGHPDKF (SEQ ID NO: 157) |
| PLP (131-159) | AHSLERVCHCLGKWLGHPDKFVGITYALT |
| | (SEQ ID NO: 158) |
| PLP (178-191)* | NTWTTCQSIAFPSK (SEQ ID NO: 159) |
| PLP (170-199) | AVPVYIYFNTWTTCQSIAFPSKTSASIGSL |
| | (SEQ ID NO: 160) |
| MBP (84-104)* | VHFFKNIVTPRTPPPSQGKGR (SEQ ID |
| | NO: 161) |
| MBP (76-112) | RTQDENPVVHFFKNIVTPRTPPPSQGKGRGL |
| | SLSRF (SEQ ID NO: 162) |

*T cell epitope-induced EAE observed

In preferred embodiments, the antigenic unit of the construct of the disclosure includes one or more T cell epitopes selected from the group consisting of MOG (35-55), MOG (27-63), PLP (139-151), PLP (131-159), PLP (178-191), PLP (170-199), MBP (84-104) and MBP (76-112). A pharmaceutical composition comprising such a construct may be used in the treatment of MS.

In some embodiments, the autoimmune disease is type 1 diabetes mellitus. In some embodiments, the self-antigen is glutamic acid decarboxylase 65-kilodalton isoform (GAD65), which is a self-antigen involved in type 1 diabetes mellitus. In some other embodiments, the self-antigen is insulin, IA-2 or ZnT8. In yet some other embodiments, the self-antigen is IGRP, ChgA, IAPP, peripherin, tetraspanin-7, GRP78, Urocortin-3 or Insulin gene enhancer protein isl-1.

In some embodiments, the autoimmune disease is celiac disease. In some embodiments, the self-antigen is α-gliadin, γ-gliadin, ω-gliadin, low molecular weight glutenin, high molecular weight glutenin, hordein, secalin or avenin b. In some embodiments, the antigenic unit comprises the T cell epitope α-gliadin (76-95).

In some embodiments, the autoimmune disease is rheumatoid arthritis. In some embodiments, the self-antigen is collagen. In some embodiments, the self-antigen is heat shock protein 60 (HSP60). In some embodiments, the self-antigen is Band 3. In some embodiments, the self-antigen is small nuclear ribonucleoprotein D1 (SmD1). In some embodiments, the self-antigen is the acetylcholine receptor (AChR). In some embodiments, the self-antigen is myelin protein zero (P0).

In some embodiments, the autoimmune disease is chronic inflammatory demyelinating polyradiculoneuropathy (CIDP) and the self-antigen is neurofascin 155. In other embodiments, the autoimmune disease is Hashimoto's thyroiditis (HT) and the self-antigen is thyroid peroxidase and/or thyroglobulin. In other embodiments, the autoimmune disease is pemphigus foliaceus and the self-antigen is desmosome-associated glycoprotein. In other embodiments, the autoimmune disease is pemphigus vulgaris and the self-antigen is desmoglein 3. In other embodiments, the autoimmune disease is thyroid eye disease (TED) and the self-antigen is calcium binding protein (calsequestrin). In other embodiments, the autoimmune disease is Grave's disease and the self-antigen is thyroid stimulating hormone receptor. In other embodiments, the autoimmune disease is primary biliary cirrhosis (PBC) and the self-antigen is antimitochondrial antibodies (AMAs), antinuclear antibodies (ANA), Rim-like/membrane (RL/M) and/or multiple nuclear dot (MND). In other embodiments, the autoimmune disease is myasthenia gravis and the self-antigen is acetylcholine receptor. In other embodiments, the autoimmune disease is insulin-resistant diabetes and the self-antigen is insulin receptor. In other embodiments, the autoimmune disease is autoimmune hemolytic anemia and the self-antigen is erythrocytes. In other embodiments, the autoimmune disease is rheumatoid arthritis and the self-antigens are citrullinated, homocitrullinated proteins and/or the Fc portion of IgG.

In other embodiments, the autoimmune disease is psoriasis and the self-antigens are cathelicidin (LL-37), disintegrin-like and metalloprotease domain containing thrombospondin type 1 motif-like 5 (ADAMTSL5), phospholipase A2 group IVD (PLA2G4D), heterogeneous nuclear ribonucleoprotein A1 (hnRNP-A1) and keratin 17.

### Unit Linker

The antigenic unit and the multimerization unit, such as a dimerization unit are preferably connected by a unit linker. The unit linker may comprise a restriction site in order to facilitate the construction of the polynucleotide. It is preferred that the unit linker is a GLGGL linker (SEQ ID NO: 90) or a GLSGL linker (SEQ ID NO: 163). In some embodiments, the unit linker comprises or consists of the nucleotide sequence as set forth in SEQ ID NO: 204.

The antigenic unit and the multimerization unit are preferably connected by a unit linker. The unit linker may comprise a restriction site in order to facilitate the construction of the polynucleotide. It is preferred that the unit linker is a GLGGL linker (SEQ ID NO: 90) or a GLSGL linker (SEQ ID NO: 163). In some embodiments, the unit linker comprises or consists of the nucleotide sequence as set forth in SEQ ID NO: 204.

The antigenic unit and the dimerization unit are preferably connected by a unit linker. The unit linker may comprise a restriction site in order to facilitate the construction of the polynucleotide. It is preferred that the unit linker is a GLGGL linker (SEQ ID NO: 90) or a GLSGL linker (SEQ ID NO: 163)). In some embodiments, the unit linker comprises or consists of the nucleotide sequence as set forth in SEQ ID NO: 204.

In some embodiments, the unit linker comprises or consists of GGGGS (SEQ ID NO: 53), GGGGSGGGGS (SEQ ID NO: 56), (GGGGS)m (SEQ ID NO: 164), EAAAK (SEQ ID NO: 144), (EAAAK)m (SEQ ID NOs: 165), (EAAAK)mGS (SEQ ID NO: 166), or (EAAK)mGS (SEQ ID NO: 31), where m is an integer greater than or equal to 1, GPSRLEEELRRRLTEPG (SEQ ID NO: 167), AAY or HEYGAEALERAG (SEQ ID NO: 168).

### Multimerization Unit and Dimerization Unit

The construct of the disclosure comprises a multimerization unit, such as a dimerization unit.

In some embodiments, the construct of the disclosure comprises a multimerization unit.

In some embodiments, the construct of the disclosure comprises a dimerization unit.

The term “multimerization unit” as used herein, refers to a sequence of nucleotides or amino acids between the antigenic unit and the targeting unit. In addition to connecting the antigenic unit and the targeting unit, the multimerization unit facilitates multimerization of/joins multiple polypeptide, such as two, three, four or more polypeptides into a multimeric protein, such as a dimeric protein, a trimeric protein or a tetrameric protein. The multimerization unit also provides the flexibility in the multimeric protein to allow optimal binding of the targeting unit to the surface molecules on the APCs, even if they are located at variable distances. The multimerization unit may be any unit that fulfils one or more of these requirements.

Multimerization Unit that Facilitates Multimerization of/Joins More than Two Polypeptides

In one embodiment, the multimerization unit is a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in one embodiment, the multimerization unit is a trimerization unit that comprises or consists of the nucleotide sequence with SEQ ID NO: 42, or an amino acid sequence encoded by said nucleotide sequence. In another embodiment, the trimerization unit is the C-terminal domain of T4 fibritin. Thus, in one embodiment, the multimerization unit is a trimerization unit that comprises or consists of the amino acid sequence with SEQ ID NO: 43, or a nucleotide sequence encoding said amino acid sequence.

In another embodiment, the multimerization unit is a tetramerization unit, such as a domain derived from p53, optionally further comprising a hinge region as described below. Thus, in one embodiment, the multimerization unit is a tetramerization unit that comprises or consists of the nucleic acid sequence with SEQ ID NO: 43, or an amino acid sequence encoded by said nucleic acid sequence, optionally further comprising a hinge region as described below.

The term “hinge region” in the context of a multimerization unit refers to an amino acid sequence comprised in the multimerization unit that contributes to joining two or more of the polypeptides, e.g. three or four polypeptides, i.e. contributes to the formation of the multimeric or dimeric protein and/or functions as a flexible spacer, allowing the targeting units of the multimeric protein to bind simultaneously to multiple surface molecules on APCs, even if these surface molecules are located at variable distances.

### Dimerization Unit

The term “dimerization unit” as used herein, refers to a sequence of nucleotides or amino acids between the antigenic unit and the targeting unit. In addition to connecting the antigenic unit and the targeting unit, the dimerization unit facilitates dimerization of/joins two polypeptides into a dimeric protein. The dimerization unit also provides the flexibility in the dimeric protein to allow optimal binding of the targeting unit to the surface molecules on the APCs, even if they are located at variable distances. The dimerization unit may be any unit that fulfils one or more of these requirements.

Accordingly, in some embodiments the construct of the disclosure comprises a dimerization unit comprising a hinge region. In other embodiments, the dimerization unit comprises a hinge region and another domain that facilitates dimerization. In yet other embodiments, the dimerization unit comprises a hinge region, a dimerization unit linker and another domain that facilitates dimerization, wherein the dimerization unit linker connects the hinge region to the other domain that facilitates dimerization. In other embodiments, the dimerization unit comprises a hinge region, a dimerization unit linker and another domain that facilitates dimerization, wherein the dimerization unit linker connects the hinge region to the other domain that facilitates dimerization. The dimerization unit linker is further described below.

In some embodiments, the dimerization unit linker is a glycine-serine rich linker, preferably GGGSSGGGSG (SEQ ID NO: 139), i.e. the dimerization unit comprises a glycine-serine rich dimerization unit linker and preferably the dimerization unit linker GGGSSGGGSG (SEQ ID NO: 139). In some embodiments, the dimerization unit linker comprises or consists of the nucleotide sequence as set forth in SEQ ID NO: 201.

The term “hinge region” refers to an amino acid sequence comprised in the dimerization unit that contributes to joining two of the polypeptides, i.e. contributes to the formation of the dimeric protein.

Moreover, the hinge region functions as a flexible spacer, allowing the two targeting units of the dimeric protein to bind simultaneously to two surface molecules on APCs, even if they are located at variable distances. The hinge region may be Ig derived, such as derived from IgG, e.g. IgG1, IgG2 or IgG3. In one embodiment, the hinge region is derived from IgM, e.g. comprising or consisting of the nucleotide sequence with SEQ ID NO: 47 or an amino acid sequence encoded by said nucleic acid sequence. The hinge region may contribute to the dimerization (or multimerization) through the formation of covalent bond(s), e.g. disulfide bridge(s) between cysteines. Thus, in some embodiments, the hinge region has the ability to form one or more covalent bonds. Preferably, the covalent bond is a disulfide bridge.

In some embodiments, the dimerization unit comprises or consists of a hinge exon h1 and hinge exon h4 (human hinge region 1 and human hinge region 4) having an amino acid sequence having at least 80% sequence identity to the amino acid sequence 1-27 of SEQ ID NO:1.

In preferred embodiments, the dimerization unit comprises or consists of a hinge exon h1 and hinge exon h4 with an amino acid sequence having at least 85% sequence identity to the amino acid sequence 1-27 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In preferred embodiments, the dimerization unit comprises or consists of a hinge exon h1 and hinge exon h4 with the amino acid sequence 1-27 of SEQ ID NO: 1, or a nucleotide sequence encoding the amino acid sequence.

In preferred embodiments, the dimerization unit comprises or consists of a hinge exon h1 and hinge exon h4 with the amino acid sequence 1-27 of SEQ ID NO: 1, except that at the most ten amino acids have been substituted, deleted or inserted, such as at the most nine amino acids, such as at the most eight amino acids, such as at the most seven amino acids, such as at the most six amino acids, such as at the most five amino acids, such as at the most four amino acids, such as at the most three amino acids, such as at the most two amino acids or such as at the most one amino acid.

In some embodiments, the dimerization unit comprises or consists of the amino acid sequence ELKTPLGDTTHT (SEQ ID NO: 156) and/or EPKSCDTPPPCPRCP (SEQ ID NO: 46), or a nucleotide sequence encoding the amino acid sequence. In some embodiments, the dimerization unit comprises or consists of a nucleotide sequence as set forth in SEQ ID NO: 200 or SEQ ID NO: 28.

In other embodiments, the dimerization unit comprises another domain that facilitates dimerization; preferably, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG. More preferably, the other domain that facilitates dimerization is a carboxyterminal C domain derived from IgG3.

In some embodiments, the dimerization unit comprises or consists of a carboxyterminal C domain derived from IgG3 with an amino acid sequence having at least 80% sequence identity to the amino acid sequence 39-144 of SEQ ID NO: 1, or a nucleotide sequence encoding the amino acid sequence.

In preferred embodiments, the dimerization unit comprises or consists of a carboxyterminal C domain derived from IgG3 with an amino acid sequence having at least 85% sequence identity to the amino acid sequence 39-144 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In preferred embodiments, the dimerization unit comprises or consists of a carboxyterminal C domain derived from IgG3 with the amino acid sequence 39-144 of SEQ ID NO: 1.

In one preferred embodiment, the dimerization unit comprises or consists of the amino acid sequence 39-144 of SEQ ID NO: 1, except that at the most 16 amino acids have been substituted, deleted or inserted, such as at the most 15, 14, 13, 12, 11, 10, 9, 8, 7, 6, 5, 4, 3, 2, or 1 amino acid.

The immunoglobulin domain contributes to dimerization through non-covalent interactions, e.g. hydrophobic interactions. Thus, in some embodiments, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In preferred embodiments, the dimerization unit comprises a hinge exon h1, a hinge exon h4, a dimerization unit linker and a CH3 domain of human IgG3. In further preferred embodiments, the dimerization unit comprises a polypeptide consisting of hinge exon h1, hinge exon h4, a dimerization unit linker and a CH3 domain of human IgG3.

In other preferred embodiments, the dimerization unit consists of a polypeptide consisting of hinge exon h1, hinge exon h4, a dimerization unit linker and a CH3 domain of human IgG3.

In some embodiments, the dimerization unit comprises an amino acid sequence having at least 80% sequence identity to the amino acid sequence of SEQ ID NO: 1.

In preferred embodiments, the dimerization unit comprises an amino acid sequence having at least 85% sequence identity to the amino acid sequence of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In more preferred embodiments the dimerization unit consists of an amino acid sequence having at least 80% sequence identity to the amino acid sequence of SEQ ID NO: 1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In even more preferred embodiments, the dimerization unit consists of the amino acid sequence of SEQ ID NO: 1, or a nucleotide sequence encoding the amino acid sequence.

In one preferred embodiment, the dimerization unit comprises the amino acid sequence of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In one preferred embodiment, the dimerization unit consists of the amino acid sequence of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In some embodiments, the dimerization unit linker is a glycine-serine rich linker, preferably GGGSSGGGSG (SEQ ID NO: 139), i.e. the dimerization unit comprises a glycine-serine rich dimerization unit linker and preferably the dimerization unit linker GGGSSGGGSG (SEQ ID NO: 139).

### Signal Peptide

In preferred embodiments, the construct of the disclosure is a polynucleotide which further comprises a nucleotide sequence encoding a signal peptide. The signal peptide is either located at the N-terminal end of the targeting unit or the C-terminal end of the targeting unit, depending on the orientation of the targeting unit in the polypeptide (FIG. 1). The signal peptide is designed to allow secretion of the polypeptide encoded by the nucleic acid comprised in the polynucleotide in the cells transfected with said polynucleotide.

Any suitable signal peptide may be used. Examples of suitable peptides are a human Ig VH signal peptide or the signal peptides which are naturally present at the N-terminus of any of the targeting units described herein, e.g. a human signal peptide of human IL-10 or a human signal peptide of human TGFβ.

Thus, in some embodiments, the polynucleotide comprises a nucleotide sequence encoding a human IL-10 signal peptide and preferably comprises a nucleotide sequence encoding a human IL-10 targeting unit. In other embodiments, the polynucleotide comprises a nucleotide sequence encoding a human Ig VH signal peptide and preferably comprises a nucleotide sequence encoding a scFv, e.g. human anti-DEC205.

In some embodiments, the polynucleotide comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%, sequence identity to the amino acid sequence of SEQ ID NO: 6 OR SEQ ID NO: 48.

In preferred embodiments, the polynucleotide comprises a nucleotide sequence encoding a signal peptide that comprises the amino acid sequence of SEQ ID NO: 6 OR SEQ ID NO: 48.

In other embodiments, the polynucleotide comprises a nucleotide sequence encoding a signal peptide that consists of an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence of SEQ ID NO: 6 OR SEQ ID NO: 48.

In other preferred embodiments, the polynucleotide which comprises a nucleotide sequence encoding a signal peptide with the amino acid sequence of SEQ ID NO: 6 OR SEQ ID NO: 48.

In other embodiments, the polynucleotide comprises a nucleotide sequence encoding a signal peptide that comprises or consists of an amino acid sequence of SEQ ID NO: 6 OR SEQ ID NO: 48, except that at the most five amino acids have been substituted, deleted or inserted, such as at the most four amino acids, such as at the most three amino acids, such as at the most two amino acids or such as at the most one amino acid.

In some embodiments, the polynucleotide comprises a nucleotide sequence encoding a murine IL-10 signal peptide, such as the IL-10 signal peptide set forth in SEQ ID NO: 50, and preferably comprises a nucleotide sequence encoding a murine IL-10 targeting unit, such as the murine IL-10 targeting unit set forth in SEQ ID NO: 169.

In some embodiments, the signal peptide is selected from the group consisting of IL-10 signal peptide, SCGB3A2 signal peptide, VSIG-3 signal peptide, CTLA4 signal peptide, or PD-1 signal peptide, such as selected from the group consisting of murine IL-10 signal peptide, murine SCGB3A2 signal peptide, murine VSIG-3 signal peptide, murine CTLA4 signal peptide, or murine PD-1 signal peptide. In some embodiments, the signal peptide comprises a sequence having 80% sequence identity to a sequence selected from the group consisting of SEQ ID NO: 50, 170, 172, 174, 176 and 178.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. AlignO does not penalize to gaps in the end of the sequences. When utilizing the ALIGN and AlignO program for comparing amino acid sequences, a BLOSUM50 substitution matrix with gap opening/extension penalties of −12/−2 is preferably used.

Amino acid sequence variants may be prepared by introducing appropriate changes into the nucleotide sequence encoding the tolerance inducing construct, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the final construct, provided that the final construct possesses the desired characteristics. For example, deletions, insertions or substitutions of amino acid residues may produce a silent change and result in a functionally equivalent peptide/polypeptide.

Deliberate amino acid substitutions may be made based on similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitution such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, aaline, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, p-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above,* indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or p-alanine residues. A further form of variation involves the presence of one or more amino acid residues in peptoid form.

### Polynucleotides

The tolerance-inducing construct of the disclosure may be in the form of a polynucleotide.

A further aspect of the disclosure is a polynucleotide a nucleotide sequence encoding a targeting unit targeting or capable of targeting antigen-presenting cells, a dimerization unit and an antigenic unit, wherein the antigenic unit comprises one or more T cell epitopes of a self-antigen, an allergen, an alloantigen or a xenoantigen.

The polynucleotide may be a DNA or RNA, including genomic DNA, cDNA and mRNA, either double stranded or single stranded. In preferred embodiments, the construct is a DNA plasmid, i.e. the polynucleotide is a DNA.

It is preferred that the polynucleotide is optimized for use in the species to which it is administered. For administration to a human, it is thus preferred that the polynucleotide sequence is human codon optimized.

### Polypeptides and Multimeric/Dimeric Proteins

The construct of the disclosure may be in the form of a polypeptide encoded by the nucleotide sequence comprised in the polynucleotide as described above.

A further aspect of the disclosure is a polypeptide, comprising a targeting unit targeting or capable of targeting antigen-presenting cells, a multimerization, such as a dimerization unit, and an antigenic unit, wherein the antigenic unit comprises one or more T cell epitopes of a self-antigen, an allergen, an alloantigen or a xenoantigen.

A further aspect of the disclosure is a polypeptide, comprising a targeting unit targeting or capable of targeting antigen-presenting cells, a multimerization and an antigenic unit, wherein the antigenic unit comprises one or more T cell epitopes of a self-antigen, an allergen, an alloantigen or a xenoantigen.

A further aspect of the disclosure is a polypeptide, comprising a targeting unit targeting or capable of targeting antigen-presenting cells, a dimerization unit and an antigenic unit, wherein the antigenic unit comprises one or more T cell epitopes of a self-antigen, an allergen, an alloantigen or a xenoantigen.

The polypeptide may be expressed in vitro for production of the tolerance-inducing construct, e.g. for production of a pharmaceutical composition comprising the construct, or the polypeptide may be expressed in vivo as a result of the administration of the polynucleotide to a subject, as described above. Due to the presence of the multimerization/dimerization unit, multimeric/dimeric proteins are formed when the polypeptide is expressed, i.e. by joining multiple polypeptides via their respective multimerization/dimerization units.

A further aspect of the disclosure is a multimeric, such as a dimeric protein, comprising multiple polypeptides, such as two polyepptides, each of which comprising a targeting unit targeting or capable of targeting antigen-presenting cells, a multimerization unit, such as a dimerization unit, and an antigenic unit, wherein the antigenic unit comprises one or more T cell epitopes of a self-antigen, an allergen, an alloantigen or a xenoantigen.

A further aspect of the disclosure is a multimeric comprising multiple polypeptides, each of which comprising a targeting unit targeting or capable of targeting antigen-presenting cells, a multimerization unit and an antigenic unit, wherein the antigenic unit comprises one or more T cell epitopes of a self-antigen, an allergen, an alloantigen or a xenoantigen.

A further aspect of the disclosure is a dimeric protein, comprising multiple polypeptides, each of which comprising a targeting unit targeting or capable of targeting antigen-presenting cells, a dimerization unit, and an antigenic unit, wherein the antigenic unit comprises one or more T cell epitopes of a self-antigen, an allergen, an alloantigen or a xenoantigen.

The multimeric protein may be a homomultimer, i.e. a multimeric protein wherein the multiple polypeptide chains are identical and consequently comprise identical units and thus identical antigen sequences, or the multimeric protein may be a heteromultimer comprising multiple polypeptide chains, wherein each polypeptide chain may comprise different antigen sequences in its antigenic unit. The dimeric protein may be a homodimer, i.e. a dimeric protein wherein the two polypeptide chains are identical and consequently comprise identical units and thus antigen sequences, or the dimeric protein may be a heterodimer comprising two polypeptide chains, wherein polypeptide chain 1 comprises different T cell epitopes in its antigenic unit than polypeptide 2. The latter may be relevant if the number of T cell epitopes for inclusion into the antigenic unit would exceed the upper size limit for the antigenic unit. It is preferred that the multimeric/dimeric protein is a homomultimeric/homodimeric protein.

### Vectors

The polynucleotide sequence of the construct may be a DNA polynucleotide comprised in a vector suitable for transfecting a host cell and expression of a polypeptide or multimeric/dimeric protein encoded by the nucleic acid sequence comprised in the polynucleotide, i.e. an expression vector, preferably a DNA plasmid. In another embodiment, the vector is suitable for transfecting a host cell and expression of an mRNA encoding for the polypeptide/multimeric protein.

A further aspect of the disclosure is a vector comprising a polynucleotide comprising a nucleotide sequence encoding a targeting unit targeting or capable of targeting antigen-presenting cells, a multimerization, such as a dimerization unit, and an antigenic unit, wherein the antigenic unit comprises one or more T cell epitopes of a self-antigen, an allergen, an alloantigen or a xenoantigen.

A further aspect of the disclosure is a vector comprising a polynucleotide comprising a nucleotide sequence encoding a targeting unit targeting or capable of targeting antigen-presenting cells, a multimerization and an antigenic unit, wherein the antigenic unit comprises one or more T cell epitopes of a self-antigen, an allergen, an alloantigen or a xenoantigen.

A further aspect of the disclosure is a vector comprising a polynucleotide comprising a nucleotide sequence encoding a targeting unit targeting or capable of targeting antigen-presenting cells, a dimerization unit, and an antigenic unit, wherein the antigenic unit comprises one or more T cell epitopes of a self-antigen, an allergen, an alloantigen or a xenoantigen.

Preferably, the vector allows for easy exchange of the various units described above, particularly the antigenic unit.

In some embodiments, the vector may be pALD-CV77 or any other vector which does not comprise bacterial nucleotide sequences which are known to trigger an immune response in an unfavourable way, when introduced into a subject. The antigenic unit may be exchanged with an antigenic unit cassette restricted by a convenient restriction enzyme, e.g. the Sfil restriction enzyme cassette where the 5′ site is incorporated in the nucleotide sequence encoding the GLGGL (SEQ ID NO: 90) and/or GLSGL(SEQ ID NO: 163) unit linker and the 3′ site is included after the stop codon in the vector.

The vectors of the disclosure may be any molecules which are suitable to carry foreign nucleic acid sequences, such as DNA or RNA, into a cell, where they can be expressed, i.e. expression vectors.

In some embodiments, the vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In other embodiments, the vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In preferred embodiments, the vector is a DNA vector, more preferably a DNA plasmid. In preferred embodiments, the vector is a DNA plasmid and the polynucleotide is DNA.

### Plasmids

A plasmid is a small, extrachromosomal DNA molecule within a cell that is physically separated from chromosomal DNA and can replicate independently. Plasmids are mostly found as small circular, double-stranded DNA molecules in bacteria; however, plasmids are sometimes present in archaea and eukaryotic organisms. Artificial plasmids are widely used as vectors in molecular cloning, serving to deliver and ensure high expression of recombinant DNA sequences within host organisms. Plasmids comprise several important features, including a feature for selection of cells comprising the plasmid, such as for example a gene for antibiotic resistance, an origin of replication, a multiple cloning site (MCS) and promoters for driving the expression of the inserted gene(s) of interest.

Generally, promoters are sequences capable of attracting initiation factors and polymerases to the promoter, so that a gene is transcribed. Promoters are located near the transcription start sites of genes, upstream on the DNA. Promoters can be about 100-1000 base pairs long. The nature of the promoter is usually dependent on the gene and product of transcription and type or class of RNA polymerase recruited to the site. When the RNA polymerase reads the DNA of the plasmid, an RNA molecule is transcribed. After processing, the mRNA will be able to be translated numerous times, and thus result in many copies of the proteins encoded by the genes of interest, when the ribosome translates the mRNA into protein. Generally, the ribosome facilitates decoding by inducing the binding of complementary tRNA anticodon sequences to mRNA codons. The tRNAs carry specific amino acids that are chained together into a polypeptide as the mRNA passes through and is “read” by the ribosome. Translation proceeds in three phases, initiation, elongation, and termination. Following the translation process, the polypeptide folds into an active protein and performs its functions in the cell or is exported from the cell and performs its functions elsewhere, sometimes after a considerable number of posttranslational modifications.

When a protein is destined for export out of the cell, a signal peptide directs the protein into the endoplasmic reticulum, where the signal peptide is cleaved off and the protein is transferred to the cell periphery after translation has terminated.

The DNA plasmid of the present disclosure is not limited to any specific plasmid, the skilled person will understand that any plasmid with a suitable backbone can be selected and engineered by methods known in the art to comprise the elements and units of the present disclosure.

### Host Cell

A further aspect of the disclosure is a host cell comprising
i) a polynucleotide comprising a nucleotide sequence encoding a targeting unit targeting or capable of targeting antigen-presenting cells, a multimerization unit, such as a dimerization unit, and an antigenic unit, wherein the antigenic unit comprises one or more T cell epitopes of a self-antigen, an allergen, an alloantigen or a xenoantigen; or
ii) a polypeptide encoded by the nucleic acid sequence defined in (i); or
iii) a multimeric protein, such as a dimeric protein, consisting of two polypeptides as defined in (ii), such as two polypeptides.

A further aspect of the disclosure is a host cell comprising
i) a polynucleotide comprising a nucleotide sequence encoding a targeting unit targeting or capable of targeting antigen-presenting cells, a multimerization unit and an antigenic unit, wherein the antigenic unit comprises one or more T cell epitopes of a self-antigen, an allergen, an alloantigen or a xenoantigen; or
ii) a polypeptide encoded by the nucleic acid sequence defined in (i); or
iii) a multimeric protein consisting of two polypeptides as defined in (ii).

A further aspect of the disclosure is a host cell comprising
i) a polynucleotide comprising a nucleotide sequence encoding a targeting unit targeting or capable of targeting antigen-presenting cells, a dimerization unit and an antigenic unit, wherein the antigenic unit comprises one or more T cell epitopes of a self-antigen, an allergen, an alloantigen or a xenoantigen; or
ii) a polypeptide encoded by the nucleic acid sequence defined in (i); or
iii) a dimeric protein consisting of two polypeptides as defined in (ii).

Suitable host cells include prokaryotes, yeast, insect or higher eukaryotic cells. In preferred embodiments, the host cell is a human cell, preferably the cell of a human individual suffering from an immune disease and being in need of prophylactic or therapeutic treatment with the construct of the disclosure.

### Pharmaceutical Compositions

The construct of the disclosure may be administered to a subject as a pharmaceutical composition comprising the construct, e.g. the form of a polynucleotide or multimeric/dimeric protein and a pharmaceutically acceptable carrier.

A further aspect of the disclosure is pharmaceutical composition comprising a pharmaceutically acceptable carrier and
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides as defined in (ii), such as two polypeptides;
   wherein the antigenic unit comprises one or more T cell epitopes of a self-antigen, an allergen, an alloantigen or a xenoantigen.

A further aspect of the disclosure is pharmaceutical composition comprising a pharmaceutically acceptable carrier and
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

A further aspect of the disclosure is pharmaceutical composition comprising a pharmaceutically acceptable carrier and
i) a polynucleotide comprising a nucleotide sequence encoding a targeting unit targeting or capable of targeting antigen-presenting cells, a dimerization unit, and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

Suitable pharmaceutically acceptable carriers include, but are not limited to, saline, buffered saline, such as PBS, dextrose, water, glycerol, ethanol, sterile isotonic aqueous buffers, and combinations thereof.

In some embodiments, the composition may comprise one or more adjuvants. Suitable adjuvants include, but are not limited to, dexamethasone, B subunits of enterotoxin cholera toxin (CTB), TLR2 ligands, helminth-derived excretory/secretory (ES) products, rapamycin, or vitamin D3 analogues and aryl hydrocarbon receptor ligands.

In some specific embodiments the composition may comprise a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO)4-ED. Particularly preferred poloxamines are those marketed under the registered trademarks Tetronic® 904, 704, and 304, respectively. The characteristics of these poloxamines are as follows: Tetronic®904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic®304 has a total average molecular weight of 1650, a total average weight of PPO units of 990, and a PEO percentage of about 40%.

In some embodiments, the composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In other embodiments, the composition comprises the amphiphilic block co-polymer in an amount of from 2% w/v to 5% w/v, such as about 3% w/v.

For pharmaceutical compositions comprising polynucleotides, the compositions may further comprise molecules that ease transfection of cells.

The pharmaceutical composition may be formulated in any way suitable for administration to a subject, such as a patient suffering or suspected of suffering from autoimmune diseases, allergic diseases or graft rejection, e.g. such as a liquid formulation for injection, e.g. for intradermal or intramuscular injection.

The pharmaceutical composition, comprising in some embodiments a polynucleotide as described herein, e.g. comprised in a vector, may be administered in any way suitable for administration to a subject, such as administered by intradermal, intramuscular, or subcutaneous injection, or by mucosal or epithelial application, such as intranasal or oral administration.

In preferred embodiments, the pharmaceutical composition comprises a polynucleotide as described herein, optionally comprised in a vector, and is administered by intramuscular or intradermal injection.

The pharmaceutical composition of the disclosure typically comprises the polynucleotide in a range of from 0.1 μg to 10 mg, e.g. about 0.2 μg, 0.3 μg, 0.4 μg, 0.5 μg, 0.75 μg, 1 μg, 5 μg, 10 μg, 25 μg, 50 μg, 75 μg, or more; such as from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg. The pharmaceutical composition of the disclosure typically comprises the polypeptide/dimeric protein in the range of from 5 μg to 5 mg.

The amount of polynucleotide, polypeptide, dimeric protein or multimeric protein may vary depending on whether the pharmaceutical composition is administered for prophylactic or therapeutic treatment, the severity of the immune disease in the individual suffering from it and on parameters like the age, weight, gender, medical history and pre-existing conditions.

### Methods for Preparing the Pharmaceutical Composition

Suitable methods for preparing the pharmaceutical composition or vaccine according to the disclosure are disclosed in WO 2004/076489A1, WO 2011/161244A1, WO 2013/092875A1 and WO 2017/118695A1, which are incorporated herein by reference.

In one aspect, the disclosure relates to a method for preparing a pharmaceutical composition comprising the multimeric/dimeric protein, or the polypeptide as defined above by producing the polypeptides in vitro. The in vitro synthesis of the polypeptides and proteins may be carried out by any suitable method known to the person skilled in the art, such as by peptide synthesis or expression of the polypeptide in a variety of expressions systems followed by purification.

In one aspect, the disclosure relates to a method for preparing a pharmaceutical composition comprising the multimeric protein, or the polypeptide as defined above by producing the polypeptides in vitro. The in vitro synthesis of the polypeptides and proteins may be carried out by any suitable method known to the person skilled in the art, such as by peptide synthesis or expression of the polypeptide in a variety of expressions systems followed by purification.

In one aspect, the disclosure relates to a method for preparing a pharmaceutical composition comprising the dimeric protein, or the polypeptide as defined above by producing the polypeptides in vitro. The in vitro synthesis of the polypeptides and proteins may be carried out by any suitable method known to the person skilled in the art, such as by peptide synthesis or expression of the polypeptide in a variety of expressions systems followed by purification.

Thus, a further aspect of the disclosure is a method for preparing a pharmaceutical composition which comprises a multimeric protein or dimeric protein consisting of multiple polypeptides, such as two, three, four or more polypeptides; or a polypeptide, wherein the method comprises:
a) transfecting cells with a polynucleotide comprising; a nucleotide sequence encoding a targeting unit targeting or capable of targeting antigen-presenting cells, a multimerization unit, such as a dimerization unit, and an antigenic unit, wherein the antigenic unit comprises one or more T cell epitopes of a self-antigen, an allergen, an alloantigen or a xenoantigen;
b) culturing the cells;
c) collecting and purifying the multimeric protein or dimeric protein or the polypeptide expressed from the cells; and
d) mixing the multimeric protein or dimeric protein or polypeptide obtained from step c) with a pharmaceutically acceptable carrier.

Thus, a further aspect of the disclosure is a method for preparing a pharmaceutical composition which comprises a multimeric protein consisting of multiple polypeptides, such as two, three, four or more polypeptides; or a polypeptide, wherein the method comprises:
a) transfecting cells with a polynucleotide comprising; a nucleotide sequence encoding a targeting unit targeting or capable of targeting antigen-presenting cells, a multimerization unit and an antigenic unit, wherein the antigenic unit comprises one or more T cell epitopes of a self-antigen, an allergen, an alloantigen or a xenoantigen;
b) culturing the cells;
c) collecting and purifying the multimeric protein or the polypeptide expressed from the cells; and
d) mixing the multimeric protein or polypeptide obtained from step c) with a pharmaceutically acceptable carrier.

Thus, a further aspect of the disclosure is a method for preparing a pharmaceutical composition which comprises a dimeric protein two polypeptides; or a polypeptide, wherein the method comprises:
a) transfecting cells with a polynucleotide comprising; a nucleotide sequence encoding a targeting unit targeting or capable of targeting antigen-presenting cells, a multimerization unit, such as a dimerization unit, and an antigenic unit, wherein the antigenic unit comprises one or more T cell epitopes of a self-antigen, an allergen, an alloantigen or a xenoantigen;
b) culturing the cells;
c) collecting and purifying the dimeric protein or the polypeptide expressed from the cells; and
d) mixing the dimeric protein or polypeptide obtained from step c) with a pharmaceutically acceptable carrier.

In preferred embodiments, the multimeric protein, dimeric protein or polypeptide obtained from step c) is dissolved in said pharmaceutically acceptable carrier.

In preferred embodiments, the multimeric protein or polypeptide obtained from step c) is dissolved in said pharmaceutically acceptable carrier.

In preferred embodiments, the dimeric protein or polypeptide obtained from step c) is dissolved in said pharmaceutically acceptable carrier.

Purification may be carried out according to any suitable method, such as chromatography, centrifugation, or differential solubility.

In another aspect, the disclosure relates to a method for preparing a pharmaceutical composition comprising a polynucleotide comprising a nucleotide sequence encoding a targeting unit targeting or capable of targeting antigen-presenting cells, a multimerization unit, such as a dimerization unit, and an antigenic unit wherein the antigenic unit comprises one or more T cell epitopes of a self-antigen, an allergen, an alloantigen or a xenoantigen, wherein the method comprises:
a) preparing the polynucleotide;
b) optionally cloning the polynucleotide into an expression vector; and
c) mixing the polynucleotide obtained from step a) or the vector obtained from step b) with a pharmaceutically acceptable carrier.

In another aspect the disclosure relates to a method for preparing a pharmaceutical composition comprising a polynucleotide comprising a nucleotide sequence encoding a targeting unit targeting or capable of targeting antigen-presenting cells, a multimerization unit and an antigenic unit wherein the antigenic unit comprises one or more T cell epitopes of a self-antigen, an allergen, an alloantigen or a xenoantigen, wherein the method comprises:
a) preparing the polynucleotide;
b) optionally cloning the polynucleotide into an expression vector; and
c) mixing the polynucleotide obtained from step a) or the vector obtained from step b) with a pharmaceutically acceptable carrier.

In another aspect the disclosure relates to a method for preparing a pharmaceutical composition comprising a polynucleotide comprising a nucleotide sequence encoding a targeting unit targeting or capable of targeting antigen-presenting cells, a dimerization unit and an antigenic unit wherein the antigenic unit comprises one or more T cell epitopes of a self-antigen, an allergen, an alloantigen or a xenoantigen, wherein the method comprises:
a) preparing the polynucleotide;
b) optionally cloning the polynucleotide into an expression vector; and
c) mixing the polynucleotide obtained from step a) or the vector obtained from step b) with a pharmaceutically acceptable carrier.

The polynucleotide may be prepared by any suitable method known to the skilled person. For example, the polynucleotide may be prepared by chemical synthesis using an oligonucleotide synthesizer.

In particular, nucleotide sequences encoding the targeting unit and/or the dimerization unit may be synthesized individually and then ligated into a vector backbone to produce the final polynucleotide by ligating into the vector the nucleic acid sequence encoding the antigenic unit.

In one aspect, the disclosure relates to the use of the construct, the polynucleotide, the polypeptide or the multimeric protein, such as the dimeric protein, described herein as a medicament.

In one aspect, the disclosure relates to the use of the construct, the polynucleotide, the polypeptide or the multimeric protein described herein as a medicament.

In one aspect, the disclosure relates to the use of the construct, the polynucleotide, the polypeptide or the dimeric protein described herein as a medicament.

### Treatment

The construct or pharmaceutical composition of the disclosure may be used to treat autoimmune diseases, an allergic diseases or graft rejection, and treatment may either be for prophylactic or for therapeutic purpose.

The construct/pharmaceutical composition is administered such that it induces tolerance in the individual administered with such pharmaceutical composition.

Tolerance is induced by either a single administration and preferably by multiple administrations adequately spaced in time.

In a further aspect, the disclosure provides a method for treating a subject having or suspected of having an immune disease selected from the group consisting of autoimmune disease, allergic disease and graft rejection or being in need of prevention thereof, the method comprising administering to the subject a pharmaceutical composition comprising
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein consisting of multiple polypeptides as defined in (ii);
wherein the antigenic unit comprises one or more T cell epitopes of a self-antigen, an allergen, an alloantigen or a xenoantigen.

In a further aspect, the disclosure provides a method for treating a subject having or suspected of having an immune disease selected from the group consisting of autoimmune disease, allergic disease and graft rejection or being in need of prevention thereof, the method comprising administering to the subject a pharmaceutical composition comprising
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii); wherein the antigenic unit comprises one or more T cell epitopes of a self-antigen, an allergen, an alloantigen or a xenoantigen.

In a further aspect, the disclosure provides a method for treating a subject having or suspected of having an immune disease selected from the group consisting of autoimmune disease, allergic disease and graft rejection or being in need of prevention thereof, the method comprising administering to the subject a pharmaceutical composition comprising
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
wherein the antigenic unit comprises one or more T cell epitopes of a self-antigen, an allergen, an alloantigen or a xenoantigen.

The pharmaceutical composition may further comprise a pharmaceutically acceptable carrier.

In some embodiments, one dose of the pharmaceutical is administered to the subject.

In some embodiments, multiple doses of the pharmaceutical composition are administered to the subject.

In yet a further aspect, the disclosure provides a pharmaceutical composition for use in the prophylactic or therapeutic treatment of an immune disease selected from the group consisting of autoimmune disease, allergic disease and graft rejection, the pharmaceutical composition comprising:
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides as defined in (ii);
   wherein the antigenic unit comprises one or more T cell epitopes of a self-antigen, an allergen, an alloantigen or a xenoantigen.

In yet a further aspect, the disclosure provides a pharmaceutical composition for use in the prophylactic or therapeutic treatment of an immune disease selected from the group consisting of autoimmune disease, allergic disease and graft rejection, the pharmaceutical composition comprising:
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In yet a further aspect, the disclosure provides a pharmaceutical composition for use in the prophylactic or therapeutic treatment of an immune disease selected from the group consisting of autoimmune disease, allergic disease and graft rejection, the pharmaceutical composition comprising:
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

The pharmaceutical composition may further comprise a pharmaceutically acceptable carrier.

In some embodiments, one dose of the pharmaceutical is administered to the subject.

In some embodiments, multiple doses of the pharmaceutical composition are administered to the subject.

In a further aspect, the disclosure provides the use of a pharmaceutical composition for the prophylactic or therapeutic treatment of a subject suffering from or suspected of suffering from an immune disease selected from the group consisting of autoimmune disease, allergic disease and graft rejection, the method comprising administering to the subject a pharmaceutical composition comprising
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides, such as of two polyeptides, as defined in (ii);
   wherein the antigenic unit comprises one or more T cell epitopes of a self-antigen, an allergen, an alloantigen or a xenoantigen.

In a further aspect, the disclosure provides the use of a pharmaceutical composition for the prophylactic or therapeutic treatment of a subject suffering from or suspected of suffering from an immune disease selected from the group consisting of autoimmune disease, allergic disease and graft rejection, the method comprising administering to the subject a pharmaceutical composition comprising:
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In a further aspect, the disclosure provides use of a pharmaceutical composition for the prophylactic or therapeutic treatment of a subject suffering from or suspected of suffering from an immune disease selected from the group consisting of autoimmune disease, allergic disease and graft rejection, the method comprising administering to the subject a pharmaceutical composition comprising:
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

The pharmaceutical composition may further comprise a pharmaceutically acceptable carrier.

In some embodiments, one dose of the pharmaceutical is administered to the subject.

In some embodiments, multiple doses of the pharmaceutical composition are administered to the subject.

In a further aspect, the disclosure provides the use of a pharmaceutical composition for the manufacture of a medicament for the prophylactic or therapeutic treatment of an immune disease selected from the group consisting of autoimmune disease, allergic disease and graft rejection in a subject suffering from or suspected of suffering from said immune disease or being in need of prevention thereof, the medicament comprising:
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides, such as of two polypeptides, as defined in (ii);
   wherein the antigenic unit comprises one or more T cell epitopes of a self-antigen, an allergen, an alloantigen or a xenoantigen.

In a further aspect, the disclosure provides the use of a pharmaceutical composition for the manufacture of a medicament for the prophylactic or therapeutic treatment of an immune disease selected from the group consisting of autoimmune disease, allergic disease and graft rejection in a subject suffering from or suspected of suffering from said immune disease or being in need of prevention thereof, the medicament comprising
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In a further aspect, the disclosure provides the use of a pharmaceutical composition for the manufacture of a medicament for the prophylactic or therapeutic treatment of an immune disease selected from the group consisting of autoimmune disease, allergic disease and graft rejection in a subject suffering from or suspected of suffering from said immune disease or being in need of prevention thereof, the medicament comprising:
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

The pharmaceutical composition may further comprise a pharmaceutically acceptable carrier.

In some embodiments, one dose of the pharmaceutical is administered to the subject.

In some embodiments, multiple doses of the pharmaceutical composition are administered to the subject.

In a further aspect, the disclosure provides the use of a pharmaceutical composition for prophylactically or therapeutically treating a subject having or suspected of having an immune disease selected from the group consisting of autoimmune disease, allergic disease and graft rejection or being in need of prevention thereof, the treatment comprising administering to the subject a pharmaceutical composition comprising:
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides, such as of two polypeptides, as defined in (ii);
   wherein the antigenic unit comprises one or more T cell epitopes of a self-antigen, an allergen, an alloantigen or a xenoantigen.

In a further aspect, the disclosure provides the use of a pharmaceutical composition for prophylactically or therapeutically treating a subject having or suspected of having an immune disease selected from the group consisting of autoimmune disease, allergic disease and graft rejection or being in need of prevention thereof, the treatment comprising administering to the subject a pharmaceutical composition comprising:
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In a further aspect, the disclosure provides use of a pharmaceutical composition for prophylactically or therapeutically treating a subject having or suspected of having an immune disease selected from the group consisting of autoimmune disease, allergic disease and graft rejection or being in need of prevention thereof, the treatment comprising administering to the subject a pharmaceutical composition comprising:
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

The pharmaceutical composition may further comprise a pharmaceutically acceptable carrier.

In some embodiments, one dose of the pharmaceutical is administered to the subject. In some embodiments, multiple doses of the pharmaceutical composition are administered to the subject.

In a further aspect, the disclosure provides a medicament for the prophylactic or therapeutic treatment of a subject having or suspected of having an immune disease selected from the group consisting of autoimmune disease, allergic disease and graft rejection or being in need of prevention thereof, the medicament comprising
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides, such as of two polypeptides, as defined in (ii);
   wherein the antigenic unit comprises one or more T cell epitopes of a self-antigen, an allergen, an alloantigen or a xenoantigen.

In a further aspect, the disclosure provides a medicament for the prophylactic or therapeutic treatment of a subject having or suspected of having an immune disease selected from the group consisting of autoimmune disease, allergic disease and graft rejection or being in need of prevention thereof, the medicament comprising
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen.

In a further aspect, the disclosure provides a medicament for the prophylactic or therapeutic treatment of a subject having or suspected of having an immune disease selected from the group consisting of autoimmune disease, allergic disease and graft rejection or being in need of prevention thereof, the medicament comprising
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen.

The pharmaceutical composition may further comprise a pharmaceutically acceptable carrier.

In some embodiments, one dose of the pharmaceutical is administered to the subject.

In some embodiments, multiple doses of the pharmaceutical composition are administered to the subject.

In a further aspect, the disclosure provides a pharmaceutical composition comprising
i) a polynucleotide comprising a nucleotide sequence encoding a targeting unit targeting or capable of targeting antigen-presenting cells, a multimerization unit, such as a dimerization unit, and an antigenic unit; or
ii) a polypeptide encoded by the nucleic acid sequence as defined in (i); or
iii) a multimeric protein, such as a dimeric protein, consisting of multiple polypeptides, such as of two polypeptides, as defined in (ii);
   wherein the antigenic unit comprises one or more T cell epitopes of a self-antigen, an allergen, an alloantigen or a xenoantigen,
   when used in the prophylactic or therapeutic treatment of an immune disease selected from the group consisting of autoimmune disease, allergic disease and graft rejection.

In a further aspect, the disclosure provides a pharmaceutical composition comprising
i) a polynucleotide comprising a nucleotide sequence encoding a targeting unit targeting or capable of targeting antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
   wherein the antigenic unit comprises one or more T cell epitopes of a self-antigen, an allergen, an alloantigen or a xenoantigen,
   when used in the prophylactic or therapeutic treatment of an immune disease selected from the group consisting of autoimmune disease, allergic disease and graft rejection.

In a further aspect, the disclosure provides a pharmaceutical composition comprising
i) a polynucleotide comprising a nucleotide sequence encoding a targeting unit targeting or capable of targeting antigen-presenting cells, a dimerization unit and an antigenic unit; or
ii) a polypeptide encoded by the nucleic acid sequence as defined in (i); or
iii) a dimeric protein consisting of two polypeptides as defined in (ii);
   wherein the antigenic unit comprises one or more T cell epitopes of a self-antigen, an allergen, an alloantigen or a xenoantigen,
   when used in the prophylactic or therapeutic treatment of an immune disease selected from the group consisting of autoimmune disease, allergic disease and graft rejection.

The pharmaceutical composition may further comprise a pharmaceutically acceptable carrier.

In some embodiments, one dose of the pharmaceutical is administered to the subject.

In some embodiments, multiple doses of the pharmaceutical composition are administered to the subject.

In a further aspect, the disclosure provides a method for improving tolerance to a self-antigen, an allergen, an alloantigen or a xenoantigen using the tolerance-inducing construct according to the disclosure.

In a further aspect, the disclosure provides a method for improving tolerance to a self-antigen, an allergen, an alloantigen or a xenoantigen in a subject, the method comprising administering to the subject the tolerance-inducing construct or the pharmaceutical composition according to the disclosure.

In some embodiments, one dose of the tolerance-inducing construct or the pharmaceutical composition according to the disclosure is administered to the subject.

In some embodiments, multiple doses of the tolerance-inducing construct or the pharmaceutical composition according to the disclosure are administered to the subject.

Indicators of treatment success are known in the art, including increased levels of antigen-specific regulatory T cells, reduced levels of antigen-specific effector T cells, (and increased levels of regulatory T cells), reduced levels of effector T cells, reduced level of T cell activation in ELISPOT when stimulated with the antigenic unit/T-cell epitopes in the antigenic unit, reduced level of basophil activation in a basophil activation test (BAT).

A radioallergosorbent test (RAST) may likewise be used to compare the allergen-specific IgE antibody level in a blood sample from a subject before and after administration of the immunotherapy construct, wherein a lower allergen-specific IgE antibody level indicates successful tolerance induction.

### EXAMPLES

### Example 1: Design and Production of Vectors According to the Disclosure

All gene sequences described in Examples 1a, 1b and 1c were ordered from GenScript (New Jersey, US) cloned into the expression vector pALD-CV77.

### Example 1a: Design and Production of Vectors According to the Disclosure, for Use in the Treatment of Multiple Sclerosis

Myelin oligodendrocyte glycoprotein (MOG) is a protein located in the central nervous system. The immunodominant 35-55 epitope of MOG (MOG 35-55) is a primary target for both cellular and humoral immune responses during Multiple sclerosis. MOG(35-55)-induced experimental autoimmune encephalomyelitis (EAE) is the most commonly used animal model of multiple sclerosis (Hunterman, H. et al. 2022).

DNA vectors were designed, comprising nucleotide sequences encoding the following units/parts as described in Table 1.

| | | | | | Amino |
|---|---|---|---|---|---|
| | Signal | Targeting | Dimerization | Antigenic | acid |
| Construct ID | peptide | Unit | unit | Unit | sequence: |
| VB5001b | Murine Ig | NA | NA | MOG (27- | SEQ ID |
| | VH signal | | | 63)* | NO: 37 |
| | peptide | | | | |
| VB5051 | Murine Ig | NA | NA | MOG (27- | SEQ ID |
| | VH signal | | | 63) | NO: 24 |
| | peptide | | | | |
| VB5002b | Natural | Human | Hinge-region | MOG (27- | SEQ ID |
| | leader | CCL3L1 | 1 from | 63)* | NO: 38 |
| | sequence | | human IgG3 | | |
| | human | | Hinge-region | | |
| | CCL3L1 | | 4 from | | |
| VB5052 | Natural | Human | human IgG3 | MOG (27- | SEQ ID |
| | leader | CCL3L1 | Glycine- | 63) | NO: 18 |
| | sequence | | serine-linker. | | |
| | human | | Human IgG3 | | |
| | CCL3L1 | | CH3 domain. | | |
| VB5003b | Murine Ig | scFv with | Unit linker: | MOG (35- | SEQ ID |
| | VH signal | with | Glycine- | 55) | NO: 13 |
| | peptide | specificity | leucine | | |
| | | for murine | linker | | |
| | | CD205 | | | |
| VB5004b | Murine lg | scFv with | | MOG (27- | SEQ ID |
| | VH signal | with | | 63)* | NO: 39 |
| | peptide | specificity | | | |
| | | for murine | | | |
| | | CD205 | | | |
| VB5005b | Natural | Murine IL- | | MOG (35 - | SEQ ID |
| | leader | 10 | | 55) | NO: 14 |
| | sequence | | | | |
| | murine IL- | | | | |
| | 10 | | | | |
| VB5006b | Natural | Murine IL- | | MOG (27- | SEQ ID |
| | leader | 10 | | 63)* | NO: 40 |
| | sequence | | | | |
| | murine IL- | | | | |
| | 10 | | | | |
| VB5012b | Natural | Murine | | MOG (35- | SEQ ID |
| | leader | SCGB3A2 | | 55) | NO: 41 |
| | sequence | | | | |
| | murine | | | | |
| | SCGB3A2 | | | | |
| VB5046 | Natural | Murine | | MOG (27- | SEQ ID |
| | leader | VSIG-3** | | 63) | NO: 16 |
| | sequence | | | | |
| | murine | | | | |
| | VSIG-3 | | | | |
| VB50048 | Murine Ig | scFv with | | MOG (27- | SEQ ID |
| | VH signal | with | | 63) | NO: 17 |
| | peptide | specificity | | | |
| | | for murine | | | |
| | | CD205 | | | |
| VB5058 | Natural | Murine IL- | | MOG (27- | SEQ ID |
| | leader | 10 | | 63) | NO: 19 |
| | sequence | | | | |
| | murine IL- | | | | |
| | 10 | | | | |
| VB5059 | Natural | Murine | | MOG (27- | SEQ ID |
| | leader | TGFβ1 | | 63) | NO: 20 |
| | sequence | | | | |
| | murine | | | | |
| | TGFB1 | | | | |
| VB5060 | Natural | Murine | | MOG (27- | SEQ ID |
| | leader | SCGB3A2 | | 63) | NO: 21 |
| | sequence | | | | |
| | murine | | | | |
| | SCGB3A2 | | | | |
| VB5061 | Natural | Murine | | MOG (27- | SEQ ID |
| | leader | CTLA-4** | | 63) | NO: 22 |
| | sequence | | | | |
| | murine | | | | |
| | CTLA4 | | | | |
| VB5071 | Natural | Murine | | MOG (27- | SEQ ID |
| | leader | PD-1** | | 63) | NO: 23 |
| | sequence | | | | |
| | murine | | | | |
| | PD-1 | | | | |

*The murine MOG (27-63) sequence was obtained from Krienke et al. 2021 and US Patent Application US2020061166A1.
**Extracellular domain

The DNA vectors VB5003b, VB5004b, VB5005b, VB5006b, VB5012b, VB5046, VB5048, VB5058, VB5059, VB5060, VB5061 and VB5071 encode tolerance-inducing constructs comprising a targeting unit, a dimerization unit and an antigenic unit as specified in Table 1. The murine MOG (27-63) antigenic unit comprises the T-cell epitope MOG(35-55).

The DNA vectors VB5002b and VB5052 encode constructs (“Vaccibodies”) comprising a human CCL3L1 targeting unit, which is known to target APCs in a pro-inflammatory manner, i.e. constructs comprising such a targeting unit will induce enhanced immune response in subjects to which they are administered and it is expected that this compound induces an activated immune response with increased IFN-γ production (see for instance WO2011161244 A1).

The DNA vectors VB5001 b and VB5051 encode no targeting unit or dimerization unit, only MOG(27-63) as antigenic unit, i.e. a single protein/peptide.

### Example 1b: Design and Production of Vectors According to the Disclosure, for Use in the Treatment of Diabetes Mellitus

Glutamic acid decarboxylase 65 (GAD65) is considered a major autoantigen in diabetes. The peptide GAD65(201-220) gave the greatest T-cell response in transgenic mice expressing the major histocompatibility complex class II allele HLA-DQ8, after immunization with GAD65. GAD65(206-220) is an immunodominant T-cell epitope of GAD65 in NOD mice (Liu, J. et al. 1999).

DNA vectors were designed, comprising nucleotide sequences encoding the following units/parts as described in Table 2.

TABLE 2

| | | | | Anti- | Amino |
|---|---|---|---|---|---|
| Construct | Signal | Targeting | Dimerization | genic | acid |
| ID | peptide | Unit | unit | Unit | sequence: |
| VB5016b | Murine Ig | scFv with | Hinge-region 1 | GAD65 | SEQ ID |
| | VH signal | with | from human | (202- | NO: 27 |
| | peptide | specificity | IgG3 | 221) | |
| | | for murine | Hinge-region 4 | | |
| | | CD205 | from human | | |
| VB5015b | Natural | Human | IgG3 | GAD65 | SEQ ID |
| | leader | CCL3L1 | Glycine-serine- | (202- | NO: 26 |
| | sequence | | linker | 221) | |
| | human | | Human IgG3 | | |
| | CCL3L1 | | CH3 domain | | |
| | | | Unit linker: | | |
| | | | Glycine-leucine | | |
| | | | linker | | |
| VB5014b | Murine Ig | NA | NA | GAD65 | SEQ ID |
| | VH signal | | | (202- | NO: 25 |
| | peptide | | | 221) | |

The DNA vector VB5016b encodes a tolerance-inducing construct comprising the targeting unit, dimerization unit and antigenic unit as stated in Table 2.

The DNA vector VB5015b encodes a construct (“a Vaccibody”) comprising a human CCL3L1 targeting unit, which is known to target APCs in a pro-inflammatory manner, i.e. constructs comprising such a targeting unit will induce an inflammatory immune response in subjects to which they are administered and it is expected that this compound induces IFN-γ production (see for instance WO2011161244 A1).

The DNA vector VB5014b encodes no targeting unit or dimerization unit, only GAD65 (202-221) as antigenic unit, i.e. a single protein/peptide.

The murine glutamic acid decarboxylase 65 (GAD65) 202-221 (SEQ ID NO: 183) antigenic unit comprises the known T-cell epitopes GAD65 (206-220) and GAD65(202-221).

### Example 1c: Design and Production of Vectors According to the Disclosure, for Use in the Treatment of Shrimp Allergy

Tropomyosin is the major allergen in shellfish. Six major T-cell epitopes were identified for tropomyosin from the species Metapenaeus ensis (Met e 1) in a Balb/c mouse model of Met e 1 hypersensitivity. Oral immunotherapy with peptides of the six T-cell epitopes effectively reduced allergic responses towards shrimp tropomyosin (Wai, C. Y. Y et al. 2015).

DNA vectors were designed, comprising nucleotide sequences encoding the following units/parts as described in Table 3.

TABLE 3

| Construct | Signal | Targeting | Dimerization | | Amino acid |
|---|---|---|---|---|---|
| ID | peptide | Unit | unit | Antigenic Unit | sequence: |
| VB5024 | Murine Ig VH | scFv with with | Hinge-region | Met e 1 (241-260) | SED ID |
| | signal | specificity for | 1 from | | NO: 32 |
| | peptide | murine | human IgG3 | | |
| | | CD205 | Hinge-region | | |
| VB5030 | Murine Ig VH | scFv with with | 4 from | Met e 1 (241-260), (210- | SED ID |
| | signal | specificity for | human IgG3 | 230), (136-155), (76- | NO: 34 |
| | peptide | murine | Glycine- | 95), (46-65), (16-35) | |
| | | CD205 | serine-linker | | |
| VB5079 | Murine Ig VH | scFv with with | Human IgG3 | Met e 1 (1-274) | SED ID |
| | signal peptide | specificity for | CH3 domain | | NO: 35 |
| | | murine | Unit linker: | | |
| | | CD205 | Glycine- | | |
| | | | leucine | | |
| | | | linker | | |

The sequences of the antigenic unit are further specified in Table 3a below.

TABLE 3a

| | |
|---|---|
| Met e 1 | SEQ ID NO: 184 |
| Met e 1(16-35) | SEQ ID NO: 185 |
| Met e 1(46-65) | SEQ ID NO: 186 |
| Met e 1(76-95) | SEQ ID NO: 187 |
| Met e 1(136-155) | SEQ ID NO: 188 |
| Met e 1(210-230) | SEQ ID NO: 189 |
| Met e 1(241-260) | SEQ ID NO: 190 |

The DNA vectors VB5024, VB5030 and VB5079 encode tolerance-inducing constructs comprising the targeting unit, dimerization unit and antigenic unit as stated in Table 3.

The Met e 1 (241-260), (210-230), (136-155), (76-95), (46-65), (16-35) antigenic unit (SEQ ID NO: 29) contains GGGGSGGGGS (SEQ ID NO: 56) linker between the T cell epitopes. The Met e 1 (1-274) antigenic unit (SEQ ID NO: 30) contains the complete Met e 1 allergen.

### Example 1d: Design and Production of DNA Constructs of the Disclosure for Use in the Treatment of Multiple Sclerosis

All gene sequences of tested constructs described in Examples 1d and 1e were ordered from Genscript (860 Centennial Ave., Piscataway, NJ 08854, USA) and cloned into the expression vectors pUMVC4a and/or pALD-CV77, apart from construct VB5017, which was cloned into the expression vector pALD-CV77 only.

Eight constructs (DNA plasmids) were designed for use in mice, comprising various murine signal peptides and targeting units, an identical murine dimerization unit and an antigenic unit which comprises a short T cell epitope of the murine multiple sclerosis (MS) autoantigen myelin oligodendrocyte glycoprotein (MOG), i.e. MOG (35-55) or a longer T cell epitope of murine MOG, i.e. MOG (27-63).

| Construct | Signal | | Antigenic | Amino acid |
|---|---|---|---|---|
| ID | peptide | Targeting Unit | Unit | sequence: |
| VB5003 | IgVH | anti-Dec205 scFv | MOG (35-55) | SED ID NO: 2 |
| VB5004 | IgVH | anti-Dec205 scFv | MOG (27-63) | SED ID NO: 3 |
| VB5005 | IL10 | IL10 | MOG (35-55) | SED ID NO: 8 |
| VB5006 | IL10 | IL10 | MOG (27-63) | SED ID NO: 9 |
| VB5009 | TGFβ1 | TGFβ1 | MOG (35-55) | SED ID NO: 10 |
| VB5012 | SCGB3A2 | SCGB3A2 | MOG (35-55) | SED ID NO: 4 |
| VB5013 | VSIG-3 | VSIG-3 | MOG (35-55) | SED ID NO: 5 |
| VB5017 | CTLA4 | CTLA4* | MOG (35-55) | SED ID NO: 11 |

*extracellular domain

### Example 1e: Design and Production of DNA Constructs of the Disclosure for Use in the Treatment of Diabetes Mellitus

One construct (DNA plasmid) was designed for use in mice, comprising a T cell epitope from the murine glutamic acid decarboxylase, GAD65 (202-221). GAD65 is an important diabetes self-antigen. The construct comprises further the elements shown in the table below (all murine proteins):

| Construct | Signal | Targeting | Antigenic | Amino acid |
|---|---|---|---|---|
| ID | peptide | Unit | Unit | sequence: |
| VB5016 | IgVH | anti-Dec205 | GAD65 | SED ID |
| | | scFv | (202-221) | NO: 7 |

### Example 1f: Design and Production of DNA Constructs of the Disclosure for Use in the Treatment of Shrimp Allergy

A construct (DNA plasmid) is designed for use in mice, comprising a T cell epitope from tropomyosin (Pan b 1 epitope). This epitope is an important shellfish allergen. The construct comprises further the elements shown in the table below (all murine proteins):

| Construct | Signal | Targeting | Antigenic | Amino acid |
|---|---|---|---|---|
| ID | peptide | Unit | Unit | sequence: |
| VB5022 | IgVH | anti-Dec205 | Pan b 1 | SED ID |
| | | scFv | (220-240) | NO: 12 |

### Example 2a: In Vitro Characterization of the Protein Expression and Secretion of the MOG Containing Constructs

The purpose of this study was to characterize protein expression and secretion of proteins encoded by the MOG containing DNA vectors post transient transfection of mammalian cells.

HEK293 cells were obtained from ATCC and transiently transfected with MOG containing DNA vectors (VB5002b, VB5003b, VB5004b, VB5005b, VB5006b and VB5012b). Briefly, 2×10⁵ cells/well were plated in 24-well tissue culture plates with 10% FBS growth medium and transfected with 1 μg of the respective DNA vector using Lipofectamine®2000 reagent under the conditions suggested by the manufacturer (Thermo Fischer Scientific). The transfected cells were maintained at 37° C. with 5% C02 for 5 days, and the cell supernatant was collected.

Expi293F cells were obtained from Thermo Fisher and transiently transfected with MOG(27-63) containing DNA vectors (VB5052, VB5046, VB5048, VB5058, VB5059, VB5060, VB5061 and VB5071). Briefly, Expi293F cells (1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified C02 cell incubator (8% CO₂, 37° C.). Supernatants were harvested 72 hours post transfection.

The expression and secretion of proteins encoded by the MOG-containing vectors were characterized by sandwich ELISA of the supernatants using antibodies against MOG (capture antibody, mouse anti-MOG antibody, 0.25 μg/ml, 100 μl/well, sc-73330, Santa Cruz Biotechnology) and hlgG CH3 domain (detection antibody, mouse anti-human IgG Fc secondary antibody, biotin, 0.1 μg/ml, 100 μl/well, 05-4240, Invitrogen).

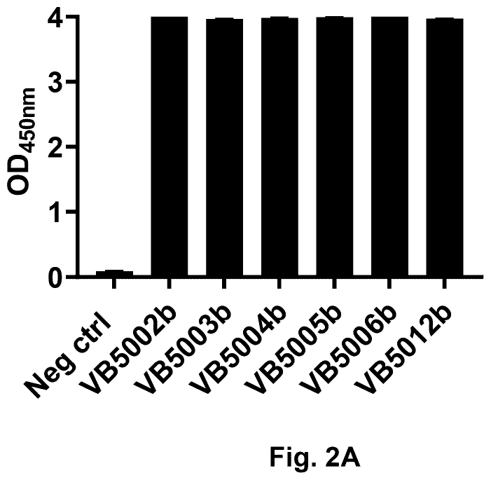

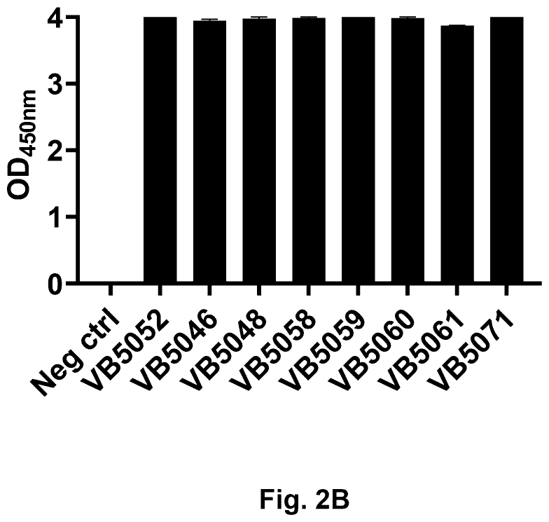

FIGS. 2A and 2B show that all the 14 MOG-containing constructs were expressed and secreted at high levels.

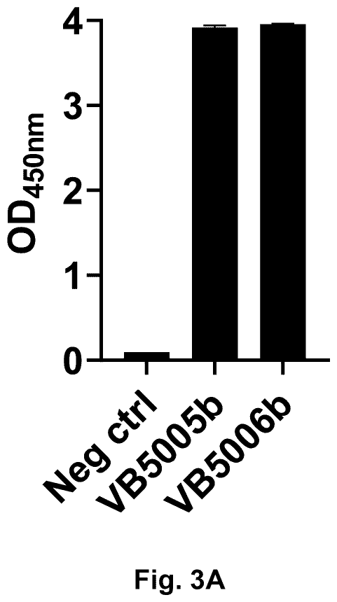

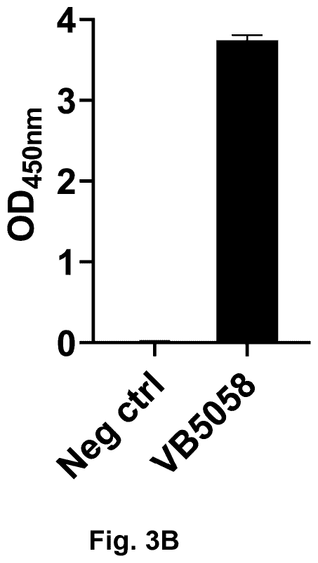

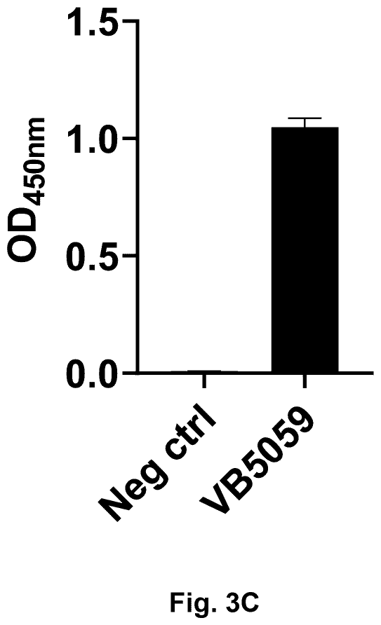

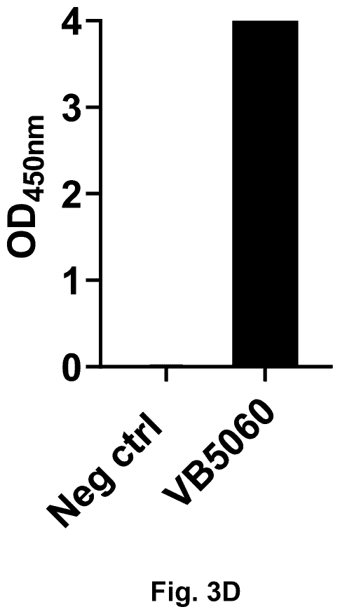

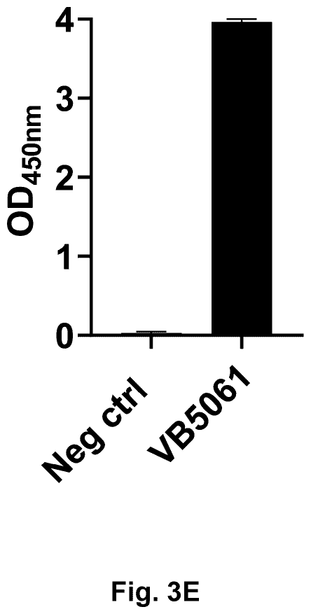

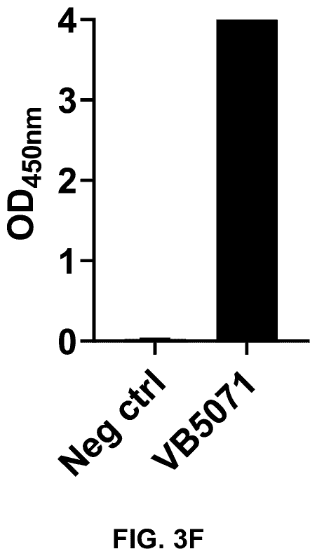

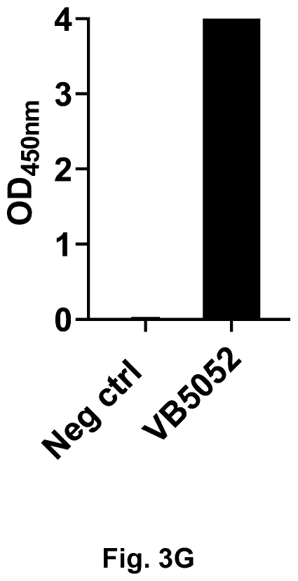

The secretion of full-length tolerance-inducing proteins with MOG antigenic unit and six different targeting units was verified by sandwich ELISA of the supernatants with antibodies against MOG and the targeting units with murine sequences of IL-10, TGFβ1, SCGB3A2, CTLA-4, PD-1 and CCL1L3, respectively. The results for the tolerance-inducing proteins are shown in FIGS. 3A-3F and the pro-inflammatory control is shown in FIG. 3G.

The detection of IL-10 (VB5005b, VB5006b and VB5058), TGFβ1 (VB5059), SCGB3A2 (VB5060), CTLA-4 (VB5061), PD-1 (VB5071) and CCL3L1 (VB5052), respectively, in combination with the detection of MOG show the secretion of full-length tolerance-inducing proteins and a pro-inflammatory control at high levels.

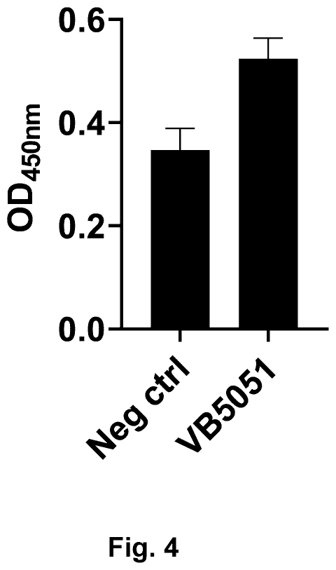

To evaluate secretion and expression of the MOG (27-63) antigen alone control peptide, encoded by the vector VB5051, Expi293F cells were transiently transfected with VB5051, and supernatant was harvested after 3 days. Secretion and expression of VB5051 was evaluated by direct ELISA, by use of the supernatant as coat, and detection using antibodies against MOG (mouse anti-MOG antibody, 3.3 μg/ml, 100 μl/well, sc-73330, Santa Cruz Biotechnology). FIG. 4 shows that the MOG (27-63) peptide is secreted upon transfection of mammalian cells with VB5051

### Example 2b: In Vitro Characterization of the Protein Expression and Secretion of a Construct of the Disclosure

The purpose of this study was to characterize the protein expression level post transient transfection of mammalian cells with the DNA plasmids, by measuring the presence of proteins in the cell supernatant by an ELISA assay using binding of specific antibodies to the targeting, dimerization and antigenic units of the protein. HEK293 cells were obtained from ATCC. HEK293 cells were transiently transfected with the constructs. Briefly, 2×10⁵ cells/well were plated in 24-well tissue culture plates with 10% FBS growth medium and transfected with 1 μg DNA plasmid using Lipofectamine®2000 reagent under the conditions suggested by the manufacturer (Invitrogen, Thermo Fischer Scientific). The transfected cells were then maintained for 6 days at 37° C. with 5% CO₂ and the cell supernatant was harvested for characterization of the protein expression.

ELISA was performed to verify the amount of protein produced by the HEK293 cells and secreted into the cell supernatant. MaxiSorp Nunc-immuno plates were coated with 0.06 μg/ml of rabbit anti human TGFbeta1 (orb77216, Biorbyte) as capture antibody in 1×PBS with 100 μl/well and plates were incubated overnight at 4° C. The microtiter wells were blocked by the addition of 200 μl/well 4% BSA in 1×PBS. 100 μl of cell supernatant from transfected HEK293 cells containing proteins expressed from the DNA plasmids were added to the plates.

For detection antibody, 1 μg/ml biotinylated mouse anti-human IgG (HP6017, Invitrogen, binds the CH3 domain of the dimerization unit included in the constructs) was added and incubated. Thereafter, SA-HRP (Streptavidin horseradish peroxidase, S2438-250UG, Sigma-Aldrich, 1:3000) was added and incubated. Unless specified, all incubations were carried out at 37° C. for 1 h, followed by 3× washing with PBS-Tween. Afterwards, 100 μl/well of TMB solution was added and color development was stopped after 5-15 min adding 100 μl/well of 1 M HCl. The optical density at 450 nm was determined on an automated plate reader (Thermo Scientific Multiscan GO).

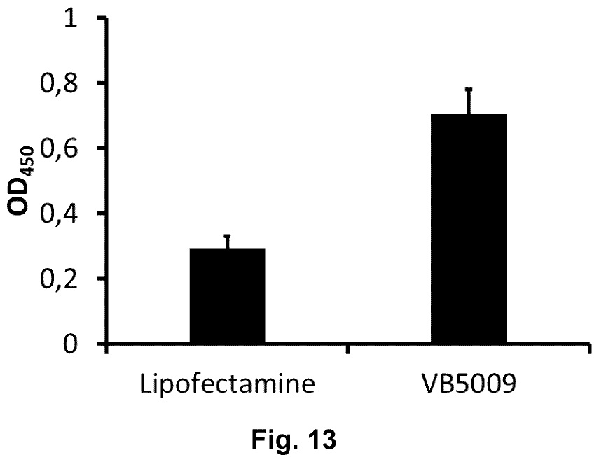

FIG. 13 shows that construct VB5009 was expressed and secreted as a protein.

### Example 3: In Vitro Characterization of the Protein Expression and Secretion of Met e 1 Containing Constructs

The purpose of this study was to characterize protein expression and secretion of tolerance-inducing proteins encoded by the Met e 1 containing DNA vectors (see Table 3) post transient transfection of mammalian cells.

Briefly, Expi293F cells (1.7×10⁶ cells/ml, 1 ml, Thermo Fisher Sci.) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified C02 cell incubator (8% C02, 37° C.). Supernatants were harvested 72 hours post transfection.

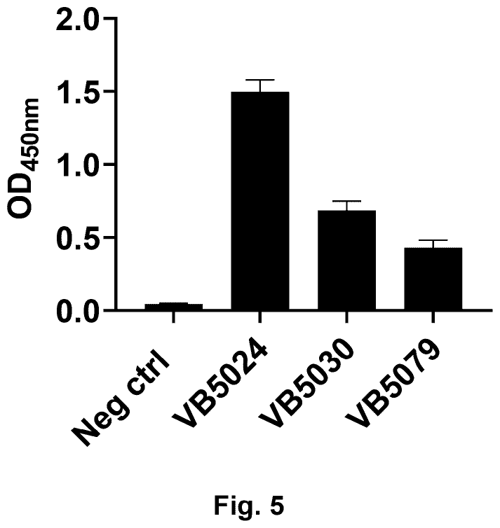

The secreted proteins encoded by the Met e 1 containing vectors were characterized by sandwich ELISA of the supernatant using antibodies against hlgG CH3 domain (capture antibody, 0.5 μg/ml mouse anti-human IgG3 (CH3 domain) antibody, 100 μl/well, MCA878G, BioRad)) and hlgG Fc domain (detection antibody, 0.250 μg/ml CaptureSelect™ Biotin Anti-IgG-Fc (Human) Conjugate, 100 μl/well, 7103262100, Invitrogen). Results are shown in FIG. 5.

As evident from FIG. 5, all the Met e 1 containing tolerance-inducing proteins (VB5024, VB5030 and VB5079) were expressed and secreted.

### Example 4: In Vitro Characterization of the Binding of a Tolerance-Inducing Construct to the DEC205 Receptor

The purpose of this experiment was to characterize functional binding of the scFv anti-DEC205 targeting unit of VB5004b (see Table 1) to recombinant DEC205 receptor. Functional binding of the targeting unit was assessed in an ELISA on supernatant from HEK293 cells transiently transfected with the tolerance-inducing DNA vector encoding the scFv anti-DEC205 as the targeting unit, by coating an ELISA-plate with recombinant DEC205 receptor and using an antibody against the antigenic unit or the dimerization unit as detection antibody.

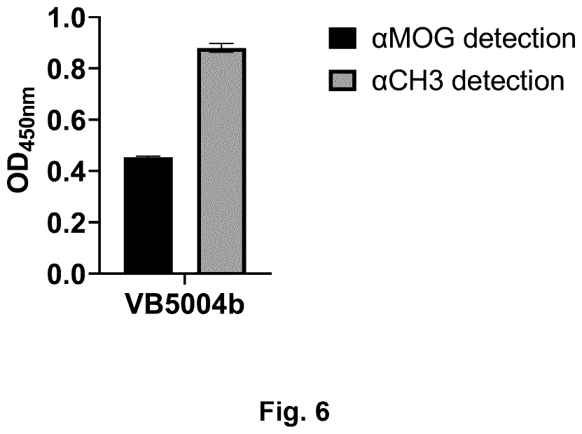

HEK293 cells were obtained from ATCC and transiently transfected with the scFv anti-DEC205 encoding DNA vector VB5004b. Briefly, 2×10⁵ HEK293 cells/well were plated in 24-well tissue culture plates with 10% FBS growth medium and transfected with 1 μg of the respective DNA vector using Lipofectamine®2000 reagent under the conditions suggested by the manufacturer (Invitrogen, Thermo Fischer Scientific). The transfected cells were maintained at 37° C. with 5% C02 for 5 days, and the cell supernatant was collected. The secreted proteins encoded by the scFv anti-DEC205 containing vector were characterized by direct ELISA of the supernatant. The ELISA plates were coated with 100 μl/well of 5 μg/ml recombinant DEC205(216-503) (OPCD05072, Aviva Systems Biology) and blocked before supernatant was added. Binding to the recombinant receptor was detected by antibodies against MOG (100 μl/well, 1 μg/ml mouse anti-MOG antibody, sc-73330, Santa Cruz Biotechnology) or hlgG CH3 domain (100 μl/well, 0.1 μg/ml mouse anti-human IgG Fc secondary antibody, biotin, 05-4240, Invitrogen). Results are shown in FIG. 6.

FIG. 6 confirms binding of the scFv anti-DEC205 containing tolerance-inducing protein, VB5004b, to the DEC205 receptor, and the secretion of full-length tolerance-inducing protein.

### Example 5: In Vitro Characterization of the Binding of Tolerance-Inducing Constructs to the IL10 Receptor

The purpose of this experiment was to characterize functional binding of the IL-10 targeting unit of VB5006b (see Table 1) to recombinant IL-10 receptor (IL-10R). Functional binding of the targeting unit was assessed in an ELISA on supernatant from HEK293 cells transiently transfected with the DNA vector encoding IL-10 as the targeting unit, by coating an ELISA-plate with recombinant IL-10 receptor and using an antibody against the antigenic unit or the dimerization unit as detection antibody.

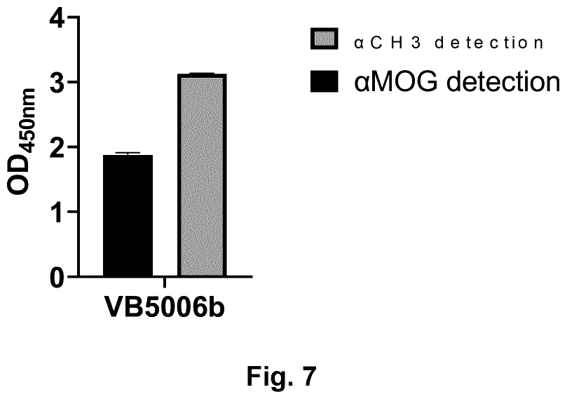

Briefly, HEK293 cells were obtained from ATCC and transiently transfected with the IL-10 containing DNA vector VB5006b. Briefly, 2×10⁵ cells/well were plated in 24-well tissue culture plates with 10% FBS growth medium and transfected with 1 μg of the respective DNA vector using Lipofectamine®2000 reagent under the conditions suggested by the manufacturer (Invitrogen, Thermo Fischer Scientific). The transfected cells were maintained at 37° C. with 5% C02 for 5 days, and the cell supernatant was collected. The secreted protein encoded by the IL-10 containing vector, was characterized by direct ELISA of the supernatant. The ELISA plates were coated with 100 μl/well of 2.5 μg/ml recombinant IL-10 receptor and blocked before supernatant was added. Binding to the recombinant receptor was detected by antibodies against MOG (100 μl/well, 1 μg/ml mouse anti-MOG antibody, sc-73330, Santa Cruz Biotechnology)) or hlgG CH3 domain (100 μl/well, 0.1 μg/ml mouse anti-human IgG Fc secondary antibody, biotin, 05-4240, Invitrogen). Results for VB5006b are shown in FIG. 7.

FIG. 7 confirms the binding of the IL10 containing tolerance-inducing protein, VB5006b, to the IL-10 receptor, and the secretion of full-length tolerance-inducing protein.

### Example 6: In Vitro Characterization of the Size and Protein Integrity of Tolerance-Inducing Constructs

Western blot analysis was performed on supernatant samples from transfected Expi293F cells to further characterize the proteins encoded by VB5046, VB5052, VB5058, VB5059, VB5061 and VB5071.

Briefly, Expi293F cells (1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). Supernatants were harvested 72 hours post transfection.

The samples were prepared by mixing 14 μl supernatant from transfected Expi293F cells with 5 μl 4× Laemmli sample buffer (Bio-Rad) with 1 μl DTT (Cayman Chemical) or 1 μl ultrapure water for reducing and non-reducing conditions, respectively (scale-up of total sample volume with the given ratio). The samples (reduced or non-reduced) were heated at 70° C. for 10 minutes before added to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with mouse anti-MOG (sc-73330, Santa Cruz Biotechnology), rat anti-murine IL-10 (MAB417, R&D Systems) or goat anti-murine CTLA-4 (AF467, R&D Systems) to detect MOG, IL-10 or CTLA-4, respectively. The membranes were incubated with fluorochrome-conjugated species-specific secondary antibodies for 1 h at room temperature, and then washed and dried. For IL-10 detection in the Dylight 488 channel, membranes were re-probed with Dylight-488 secondary antibody. Membranes were reactivated in ethanol and TBST. Membranes were blocked, incubated with Dylight 488-conjugated secondary antibodies for 1 h at room temperature, and then washed and dried. Images were acquired by using a ChemiDoc™ MP Imaging System.

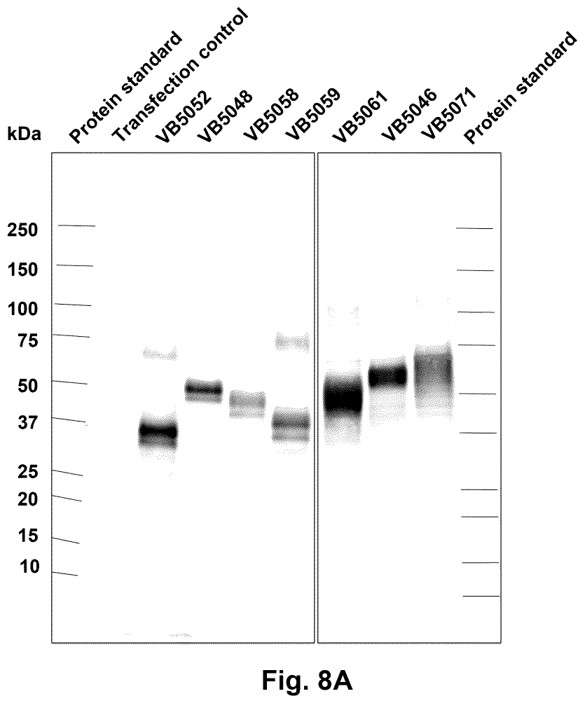

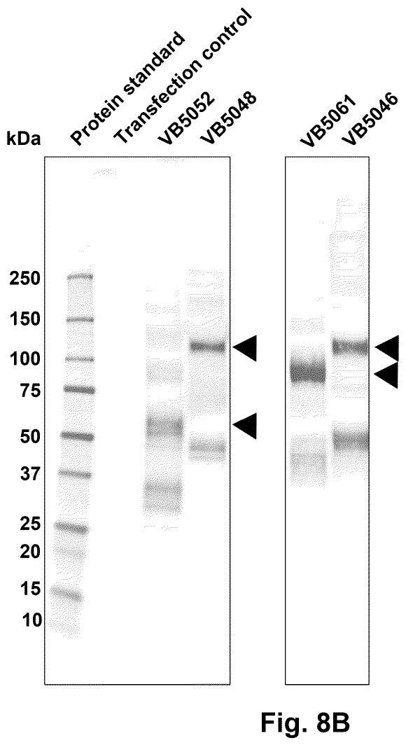

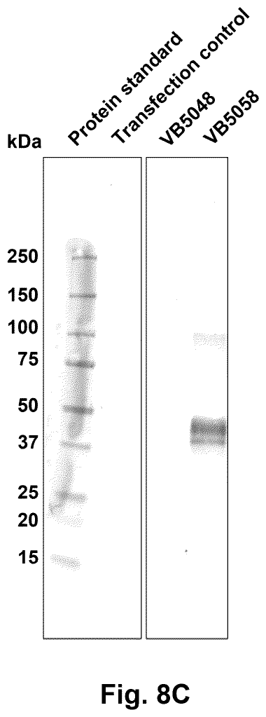

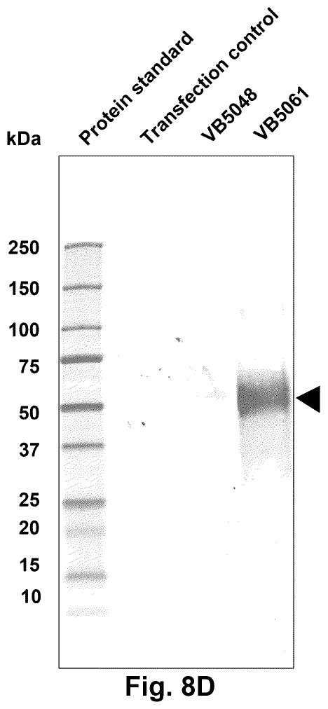

The western blot analysis with anti-MOG antibody shows full-length secretion of the tolerance-inducing proteins and VB5052 (FIG. 8A). All proteins, except VB5059, appear to have a higher molecular weight than expected based on the protein sequence, which is likely due to post translational modifications. VB5059 has TGFβ1 as targeting unit, which is known to reduce its size by 28 kDa upon cleavage into a latency-associated peptide and mature TGFβ1. FIG. 8B shows that that the proteins form dimers under non-reducing conditions. FIG. 8B also indicates the presence of the monomeric form of the protein under non-reducing conditions. The membranes probed with anti-IL-10 and anti-CTLA-4 (FIGS. 8C and 8D, respectively) displayed bands corresponding to the same molecular weight as the bands detected on the membranes probed with anti-MOG (FIG. 8A). Thus, FIGS. 8C and 8D confirm that MOG and IL-10 or CTLA-4, respectively, are parts of the same fusion protein. In FIGS. 8C and 8D VB5048 was included as a control to show that the anti-IL-10 and anti-CTLA-4, respectively, do not bind the proteins unspecifically.

### Example 7: Assessment of Tolerance Inducing Ability of a DNA Vector of the Disclosure

The tolerance-inducing ability of VB5004b (described in Table 1) was assessed in spleens from mice vaccinated with VB5004b, and determined by calculating the IL-10/IFN-γ ratio induced. The IL-10 (a non-inflammatory cytokine associated with immune tolerance) and IFN-γ (a marker for inducing an inflammatory immune response) signals were determined in a dual color FluoroSpot assay following restimulation of splenocytes harvested from vaccinated mice with MOG(35-55) peptide. The IL-10/IFN-γ ratio indicates to which extent the immune responses induced by the DNA vectors are skewed towards a tolerogenic response. A tolerogenic profile was further assessed by the frequencies of Foxp3+ cells induced, and by the lack of IFN-γ+ and IL-17+ production from CD4+ T cells after VB5004b vaccination. The results obtained were compared to the responses induced by the pro-inflammatory control vaccine VB5002b (described in Table 1) and the tolerance-inducing ability of VB5001b (described in Table 1).

### Mouse Vaccination and Fluorospot

The following study design was applied:

Female, 6-week-old C57BL/6 mice were obtained from Janvier Labs (France). All animals were housed in the animal facility at the Radium Hospital (Oslo, Norway). All animal protocols were approved by the Norwegian Food Safety Authority (Oslo, Norway). 4-5 mice/group were used for the testing of VB5004b, VB5002b and VB5001b, whereas 2 mice/group were used for the negative control (PBS only). VB5002b was included as a pro-inflammatory version of a MOG(27-63) construct, comprising a human CCL3L1 targeting unit, which is known to target APCs and induce inflammatory immune responses, i.e. constructs comprising such a targeting unit will induce a pro-inflammatory immune response in subjects to which they are administered and it is expected that this compound induces IFN-γ production in the T cells specific to the encoded antigen. A DNA vector encoding the MOG(27-63) peptide alone, VB5001b, was included as a comparison to VB5004b.

One dose of 50 μg of VB5004b or the control DNA vectors VB5001b and VB5002b dissolved in sterile PBS was administered by intramuscular needle injection to each tibialis anterior (2×25 μl, 1000 μg/ml), followed by electroporation with AgilePulse in vivo electroporation system (BTX, USA).

The spleens were harvested 7 days after vaccination and mashed in a cell strainer to obtain single cell suspensions. The red blood cells were lysed using ammonium-chloride-potassium (ACK) lysing buffer. After washing, the splenocytes were counted using NucleoCounter NC-202 (ChemoMetec, Denmark), resuspended to a final concentration of 6×10⁶ cells/ml and seeded as 6×10⁵ cells/well in a 96-well IFN-γ/IL-10 dual color FluoroSpot plate. The splenocytes were then restimulated with 16.67 μg/ml MOG (35-55) peptide for 44 hours before tested for IFN-γ and IL-10 cytokine production in a dual color FluoroSpot assay, according to the manufacturer's protocol (Mabtech AB, Sweden). Spot-forming cells were measured in an IRIS Fluorospot and ELISpot plate reader (Mabtech AB) and analyzed using the Apex software (Mabtech AB). Results are shown as the mean number of triplicates of IL-10+ or IFN-γ+ spots/10⁶ splenocytes.

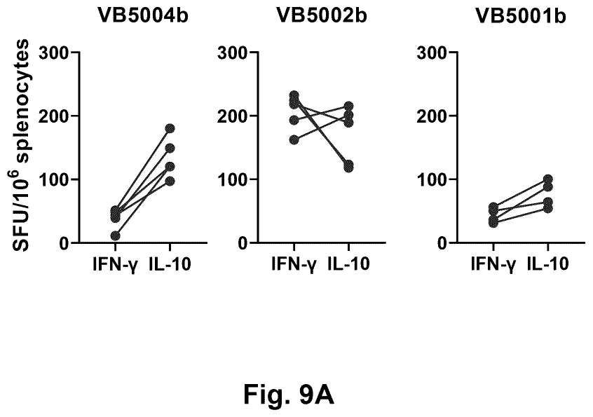

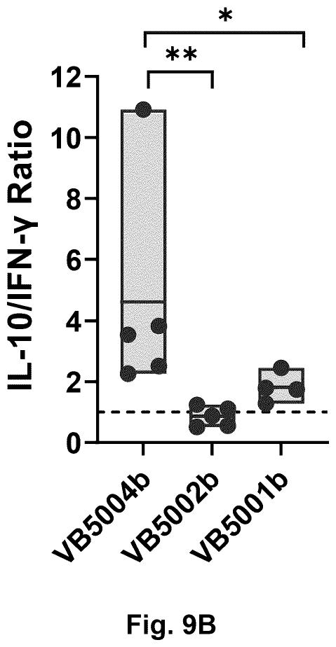

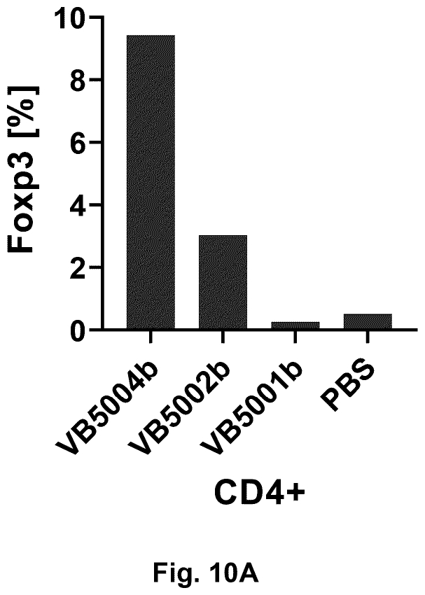

As can be seen from FIG. 9A, VB5004b induced higher levels of IL-10 compared to the levels induced by VB5001b. Further, in contrast to VB5002b which induce elevated levels of the IFN-γ, low background levels of IFN-γ were detected in response to VB5004b vaccination. FIG. 9B shows the ratio of IL-10/IFN-γ calculated from the values presented in FIG. 9A.—The IL-10/IFN-γ ratio is high for VB5004b, indicating that VB5004b induced significantly higher levels of the immunosuppressive cytokine IL-10 than the inflammatory cytokine IFN-γ. Contrary, splenocytes from mice administered with VB5002b showed an IL-10/IFN-γ ratio of about 1, indicating that both cytokines are produced in equivalent levels after re-stimulation with MOG (35-55) peptide. To avoid excess inflammation and assure eventual resolution of inflammation, it is important that the production of pro-inflammatory cytokines, such as IFN-γ, is regulated by negative feedback mechanisms—including the production of anti-inflammatory cytokines such as IL-10 (Sugimoto M A et al 2016). Therefore, the increased level of IL-10 observed in response to VB5002b may be explained by such a feedback mechanism to control the inflammatory response induced. A significantly increased IL-10/IFN-γ ratio was also detected for VB5004b compared to VB5001b, indicating a higher tolerance inducing potential of VB5004b compared to both VB5002b and VB5001b.

### Flow Cytometry

The splenocytes were further analyzed by flow cytometry for their expression of Foxp3, IFN-γ and IL-17. Foxp3 acts as a master regulator of the suppressive pathway in the development and function of regulatory T cells (Tregs).

Briefly, following 16 h re-stimulation with MOG (35-55) peptide, the cells were harvested, counted and washed before incubation with fixable viability dye (FVD) for 10 min in the dark. The cells were subsequently centrifuged and washed before surface stain mix was added: anti-CD3 (BUV395), anti-CD4 (BV785), and anti-CD8, and incubated at 4° C. in the dark for 30 min. Following the incubation, the cells were centrifuged, and the cell pellets were resuspended and washed in flow buffer (PBS, 10% FBS and 2 mM EDTA). Foxp3 fixation/permeabilization solution was added to the cells and incubated for 60 min at 4° C. in the dark. Following a centrifugation step, the cells were resuspended and washed in permeabilization buffer, before centrifuged and added intracellular antibody mix: anti-Foxp3 (Alexa fluor 700), anti-IFN-γ (APC), anti-IL-17 (Alexa fluor 488), and incubated at 4° C. in dark for 30 min. The cells were subsequently washed and resuspended in flow buffer until acquisition. Compensation was set up using single stained Ultra comp eBeads and ArC reactive beads for Fixable Viability dye. To evaluate the quality of the staining, we devised a gating strategy that excluded fluidic inconstancies, cell debris, doublets, and dead cells. We further defined T cells based on the expression of CD3. The CD3+ T cells were examined for expression of CD4 and the cells were then analyzed for Foxp3 and cytokine expression. The unstimulated cells from each group were used to evaluate background levels of cytokine production in the assay. The flow cytometry files (FCS) were exported from FACSDiva™ software and analyzed using FlowJo. Data obtained from the flow cytometry analysis were analyzed using GraphPad prism 9.

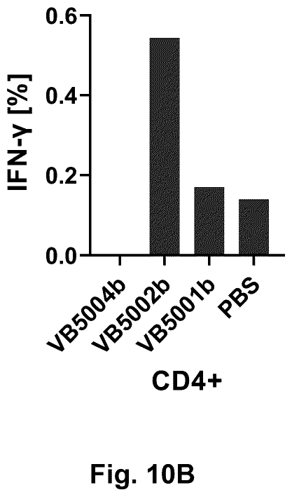

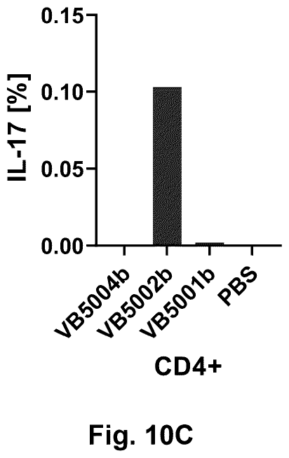

As shown in FIG. 10A, the percentage of Foxp3+ cells among the CD4+ T cells, was highly increased in mice vaccinated with VB5004b compared to the levels detected in mice vaccinated with VB5002b, VB5001b or with PBS. Both IFN-γ and IL-17 are pro-inflammatory cytokines contributing to the pathogenesis of chronic inflammatory and autoimmune diseases, including experimental autoimmune encephalomyelitis (EAE) and multiple sclerosis (Gobel K et al 2018). Thus, a tolerogenic vaccine must reliably induce tolerance without inadvertently sensitizing auto-antigen immune responses, e.g. by inducing pro-inflammatory cytokines, that may exacerbate autoimmunity. As seen in FIGS. 10B and 10C, elevated IFN-γ and IL-17 expression was detected in response to VB5002b, while lack of these pro-inflammatory cytokines was confirmed in mice vaccinated with VB5004b.

Example 7 thus shows that VB5004b, encoding the scFv anti-DEC205 targeting unit and MOG (27-63) antigenic unit, induced a higher non-inflammatory to inflammatory cytokine ratio (IL-10/IFN-γ) and shows a lack of inflammatory cytokine production compared to its pro-inflammatory version VB5002b. Example 7 further shows that VB5004b induces a higher frequency of Foxp3+CD4+ T cells compared to VB5001b, VB5002b and the background level in PBS vaccinated mice, indicating higher presence of Tregs induced by VB5004b compared to controls.

### Example 8: Assessment of Tolerance Inducing Ability of DNA Vectors According to the Disclosure

The tolerance-inducing ability of VB5012b (described in Table 1) was determined by calculating the ratio of IL-10/IFN-γ as described in Example 7. A tolerogenic profile of VB5012b, VB5048, VB5058 and VB5046 (all constructs described in Table 1) was further assessed by the percentage of MOG (38-49)-specific Foxp3+ T cells induced in response to vaccination.

### Mouse Vaccination and Fluorospot

The following study design was applied:

Female, 6-week-old C57BL/6 mice were obtained from Janvier Labs (France). All animals were housed in the animal facility at the Radium Hospital (Oslo, Norway). All animal protocols were approved by the Norwegian Food Safety Authority (Oslo, Norway). 5 mice/group were used for the testing of VB5012b, VB5048, VB5006b, VB5046, VB5052 and VB5051, whereas 2 mice/group were used for the negative control (PBS vaccinated mice only). As in Example 7, VB5052 was included as a pro-inflammatory MOG(27-63) encoding construct while VB5051 was included as a MOG(27-63) encoding antigen alone control for comparison to VB5012b, VB5048, VB5006b and VB5046.

A dose of 50 μg of the DNA vectors VB5012b, VB5048, VB5006b, VB5046, VB5051 or VB5052 was administered intramuscularly twice (day 0 and day 4) followed by electroporation, and the spleens were harvested 10 days after the first vaccination and mashed in a cell strainer to obtain single cell suspensions, as described in Example 7. Splenocytes were restimulated with MOG(35-55) peptide for 44 hours and tested for IFN-γ and IL-10 cytokine production in a dual color FluoroSpot assay, as described in Example 7.

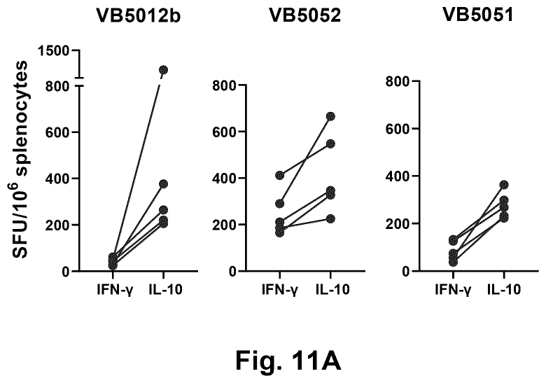

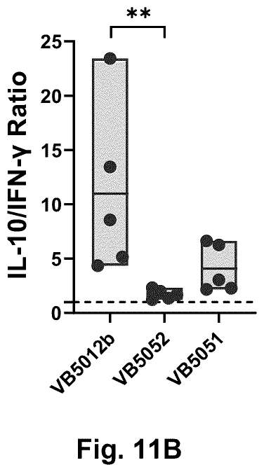

FIG. 11A shows elevated levels of IL-10 produced in spleens of mice vaccinated with VB5012b along with the lack of IFN-γ production upon restimulation with MOG(35-55) peptide. This is in contrast to VB5052 that shows increased IFN-γ levels in splenocytes from vaccinated mice. As shown in FIG. 11B, a significantly higher IL-10/IFN-γ ratio was detected for VB5012b compared to VB5052, and a higher IL-10/IFN-γ ratio tendency was detected compared to VB5051.

Flow cytometry analysis of MOG (38-49)-specific CD4′ T cells in splenocytes from vaccinated mouse using tetramer (H-2IAb/GWYRSPFSRVVH)

The generation of MOG-specific Foxp3+ cells, indicating Tregs cells that act to suppress and control MOG-specific inflammatory immune responses, and thereby maintaining self-tolerance, was identified by MOG-specific tetramer staining and flow cytometry (CD4+Foxp3+MOG(38-49)-tet+ cells).

Briefly, 2×10⁶ splenocytes pooled from each group were transferred to 96 well V bottom plates. Tetramers and Abs were diluted in PBS with 5% FBS before use and protected from light. All steps that required cell wash were performed with PBS with 5% FBS unless otherwise stated. First, the cells were stained with ProT2® MHC Class II Tetramers specific for MOG (38-49) (1 μg/ml, H-2 IAb-GWYRSPFSRVVH-ProT2® Tetramer PE, 2958, Proimmune) and the plates were incubated in a humidified CO₂ cell incubator (5% CO₂, 37° C.) for 2 h. Without washing the cells, FC receptors were blocked on ice for 5 min to prevent non-specific binding of flowcytometry antibodies (Ab) to the Fc receptor (0.25 μg/ml, TruStain FcX™ PLUS (anti-mouse CD16/32) Antibody, 156604, Biolegend). Without washing the cells, the cells were stained for 30 min on ice with surface Ab cocktail containing Anti-Mouse CD8 PE-Cy7 (0.25 μg/ml, Clone: 53-6.7, 100721, BD Biosciences), Anti-Mouse CD4 eFluor450 (0.25 μg/ml, Clone: GK1.5, 48-0041-82, Thermofischer/eBioscience), Anti-Mouse CD25 PerCP-Cy5.5 (0.25 μg/ml, Clone: PC61, 102030, Biolegend). The cells were washed twice with PBS. Next, the cells were stained on ice for 10 min with fixable viability dye (150 μl per well, 1:8000 dilution in PBS, Fixable Viability Stain 780, 565388, BD biosciences). The cells were washed twice with only PBS and fixed and permeabilized using Foxp3/Transcription Factor Staining Buffer Set according to the manufacturer's instruction (200 μl per well, 00-5523-00, Thermofischer/eBioscience). The cells were washed and stained for 30 min on ice with intracellular Ab cocktail containing Anti-Mouse FOXP3 eFluor 660 (0.25 μg/ml, Clone: FJK-16s, 50-5773-82, Thermofischer/eBioscience), Anti-Mouse Ki-67 Alexa Fluor 488 (0.25 μg/ml, Clone: Clone: 11F6, 151204, Biolegend). The cells were washed and resuspended in 150 μl of PBS with 5% FBS and analyzed with BD FACSymphony™ A3 Cell Analyzer. The following controls were used as a guide for gating desired population using FlowJo™ v10.8 Software (BD Life Sciences), Unstained controls (=cells did not receive any Ab) and Fluorescence Minus One (FMO) controls (=samples stained with all the fluorophore-labelled Abs, minus one of them to accurately discriminating positive versus negative signals).

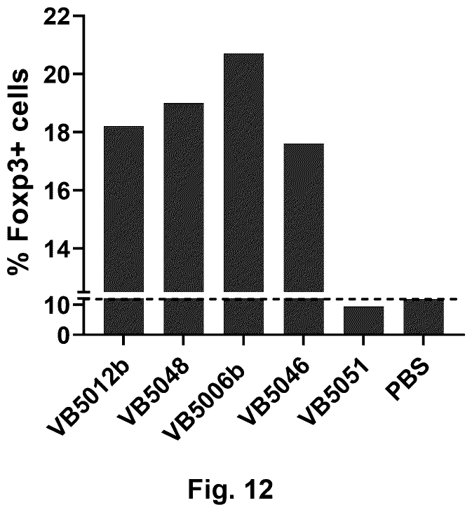

As shown in FIG. 12, a higher percentage of MOG (38-49)-specific Foxp3+ cells can be observed following the two-dose vaccination regimen (day 0+day 4) with tolerance-inducing constructs VB5012b, VB5048, VB5006b and VB5046 as compared to vaccination with VB5051 or PBS.

Example 8 thus shows that VB5012b, encoding the MARCO ligand SCGB3A2 as targeting unit, and MOG (27-63) as antigenic unit, induced a higher non-inflammatory to inflammatory cytokine ratio (IL-10/IFN-γ) compared to the pro-inflammatory version VB5052. Example 8 further shows that vaccination with the tolerance-inducing constructs VB5012b, VB5048, VB5006b or VB5046 induces a higher percentage of MOG (38-49)-specific Foxp3+ cells, indicating higher levels of Tregs induced, compared to vaccination with VB5051 and the background level in PBS vaccinated mice. These results indicate that the DNA vectors VB5048, VB5012b, VB5006b and VB5046 can elicit tolerogenic responses.

Sequences
SEQ ID NO: 1
ELKTPLGDTTHTEPKSCDTPPPCPRCPGGGSSGGGSGGQPREPQVYTLPP
SREEMTKNQVSLTCLVKGFYPSDIAVEWESSGQPENNYNTTPPMLDSDGS
FFLYSKLTVDKSRWQQGNIFSCSVMHEALHNRFTQKSLSLSPGK
VB5003
Murine IgVH signal peptide (1-19),
murine anti-DEC205 scFv (20-265),
dimerization unit (266-442), unit
linker (443-447), murine MOG 35-55
(448-468)
SEQ ID NO: 2
M¹NFGLRLIFLVLTLKGVQC¹⁹D²⁰IQMTQSPSFLSTSLGNSITITCHAS
QNIKGWLAWYQQKSGNAPQLLIYKASSLQSGVPSRFSGSGSGTDYIFTIS
NLQPEDIATYYCQHYQSFPWTFGGGTKLELKGGGGSGGGGSGGGGSEVKL
LESGGGLVQPGGSLRLSCAASGFTFNDFYMNWIRQPPGQAPEWLGVIRNK
GNGYTTEVNTSVKGRFTISRDNTQNILYLQMNSLRAEDTAIYYCARGGPY
YYSGDDAPYWGQGVMVTVSS²⁶⁵P²⁶⁶SVIFLTKRGRQVCADPSEEWVQK
YVSDLELSAELKTPLGDTTHTEPKSCDTPPPCPRCPGGGSSGGGSGGQPR
EPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLS
PGK⁴⁴²G⁴⁴³LGGL⁴⁴⁷M²⁴⁸EVGWYRSPFSRVVHLYRNGK⁴⁶⁸
VB5004
Murine IgVH signal peptide (1-19),
murine anti-DEC205 scFv (20-265),
dimerization unit (266-442), unit
linker (443-447), murine MOG 27-63
(448-484)
SEQ ID NO: 3
M¹NFGLRLIFLVLTLKGVQC¹⁹DIQMTQSPSFLSTSLGNSITITCHASQN
IKGWLAWYQQKSGNAPQLLIYKASSLQSGVPSRFSGSGSGTDYIFTISNL
QPEDIATYYCQHYQSFPWTFGGGTKLELKGGGGSGGGGSGGGGSEVKLLE
SGGGLVQPGGSLRLSCAASGFTFNDFYMNWIRQPPGQAPEWLGVIRNKGN
GYTTEVNTSVKGRFTISRDNTQNILYLQMNSLRAEDTAIYYCARGGPYYY
SGDDAPYWGQGVMVTVSS²⁶⁵P²⁶⁶SVIFLTKRGRQVCADPSEEWVQKYV
SDLELSAELKTPLGDTTHTEPKSCDTPPPCPRCPGGGSSGGGSGGQPREP
QVYTLPPSREEMTKNQVSLTCLVKGFYPSDIAVEWESSGQPENNYNTTPP
MLDSDGSFFLYSKLTVDKSRWQQGNIFSCSVMHEALHNRFTQKSLSLSPG
K⁴⁴²G⁴⁴³LGGL⁴⁴⁷S⁴⁴⁸PGKNATGMEVGWYRSPFSRVVHLYRNGKDQD
AEAQP⁴⁸⁴
VB5012
Murine SCGB3A2 signal peptide (1-21),
murine SCGB3A2 (22-91), dimerization
unit (92-268), unit linker (269-273),
murine MOG 35-55 (274-294)
SEQ ID NO: 4
M¹KLVSIFLLVTIGICGYSATA²¹L²²LINRLPVVDKLPVPLDDIIPSFD
PLKMLLKTLGISVEHLVTGLKKCVDELGPEASEAVKKLLEALSHLV⁹¹
P⁹²SVIFLTKRGRQVCADPSEEWVQKYVSDLELSAELKTPLGDTTHTEPK
SCDTPPPCPRCPGGGSSGGGSGGQPREPQVYTLPPSREEMTKNQVSLTCL
VKGFYPSDIAVEWESSGQPENNYNTTPPMLDSDGSFFLYSKLTVDKSRWQ
QGNIFSCSVMHEALHNRFTQKSLSLSPGK²⁶⁸G²⁶⁹LGGL²⁷³M²⁷⁴EVG
WYRSPFSRVVHLYRNGK²⁹⁴
VB5013
Murine VSIG-3 signal peptide (1-22),
murine VSIG-3 (23-428), dimerization
unit (429-605), unit linker (606-610),
murine MOG 35-55 (611-631)
SEQ ID NO: 5
M¹TRRRSAPASWLLVSLLGVATS²²L²³EVSESPGSVQVARGQTAVLPCA
FSTSAALLNLNVIWMVIPLSNANQPEQVILYQGGQMFDGALRFHGRVGFT
GTMPATNVSIFINNTQLSDTGTYQCLVNNLPDRGGRNIGVTGLTVLVPPS
APQCQIQGSQDLGSDVILLCSSEEGIPRPTYLWEKLDNTLKLPPTATQDQ
VQGTVTIRNISALSSGLYQCVASNAIGTSTCLLDLQVISPQPRSVGVIAG
AVGTGAVLIVICLALISGAFFYWRSKNKEEEEEEIPNEIREDDLPPKCSS
AKAFHTEISSSENNTLTSSNTYNSRYWNNNPKPHRNTESFNHFSDLRQSF
SGNAVIPSIYANGNHLVLGPHKTLVVTANRGSSPQVLPRNNGSVSRKPWP
QHTHSYTVSQMTLERIGAVPVMVPAQSRAGSLV⁴²⁸P⁴²⁹SVIFLTKRGR
QVCADPSEEWVQKYVSDLELSAELKTPLGDTTHTEPKSCDTPPPCPRCPG
GGSSGGGSGGQPREPQVYTLPPSREEMTKNQVSLTCLVKGFYPSDIAVEW
ESSGQPENNYNTTPPMLDSDGSFFLYSKLTVDKSRWQQGNIFSCSVMHEA
LHNRFTQKSLSLSPGKG⁶⁰⁶LGGL⁶¹⁰M⁶¹¹EVGWYRSPFSRWHLYRNG
K⁶³¹
Murine IgVH signal peptide (1-19)
SEQ ID NO: 6
MNFGLRLIFLVLTLKGVQC
VB5016
Murine IgVH signal peptide (1-19),
murine anti-DEC205 scFv (20-265),
dimerization unit (266-442), unit
linker (443-447), murine GAD65
202-221 (448-467)
SEQ ID NO: 7
M¹NFGLRLIFLVLTLKGVQC¹⁹D²⁰IQMTQSPSFLSTSLGNSITITCHAS
QNIKGWLAWYQQKSGNAPQLLIYKASSLQSGVPSRFSGSGSGTDYIFTIS
NLQPEDIATYYCQHYQSFPWTFGGGTKLELKGGGGSGGGGSGGGGSEVKL
LESGGGLVQPGGSLRLSCAASGFTFNDFYMNWIRQPPGQAPEWLGVIRNK
GNGYTTEVNTSVKGRFTISRDNTQNILYLQMNSLRAEDTAIYYCARGGPY
YYSGDDAPYWGQGVMVTVSS²⁶⁵P²⁶⁶SVIFLTKRGRQVCADPSEEWVQK
YVSDLELSAELKTPLGDTTHTEPKSCDTPPPCPRCPGGGSSGGGSGGQPR
EPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLS
PGK⁴⁴²G⁴⁴³LGGL⁴⁴⁷T⁴⁴⁸NMFTYEIAPVFVLLEYVTL⁴⁶⁷
VB5005
Murine IL10 signal peptide (1-18),
murine IL10 (2-178), dimerization
unit (179-355), unit linker
(356-360), murine MOG 35-55
(361-381)
SEQ ID NO: 8
M¹PGSALLCCLLLLTGMR^{I18}SRGQYSREDNNCTHFPVGQSHMLLELRTA
FSQVKTFFQTKDQLDNILLTDSLMQDFKGYLGCQALSEMIQFYLVEVMPQ
AEKHGPEIKEHLNSLGEKLKTLRMRLRRCHRFLPCENKSKAVEQVKSDFN
KLQDQGVYKAMNEFDIFINCIEAYMMIKMKS¹⁷⁸P¹⁷⁹SVIFLTKRGRQV
CADPSEEWVQKYVSDLELSAELKTPLGDTTHTEPKSCDTPPPCPRCPGGG
SSGGGSGGQPREPQVYTLPPSREEMTKNQVSLTCLVKGFYPSDIAVEWES
SGQPENNYNTTPPMLDSDGSFFLYSKLTVDKSRWQQGNIFSCSVMHEALH
NRFTQKSLSLSPGK³⁵5G³⁵⁶LGGL³⁶⁰M³⁶¹EVGWYRSPFSRVVHLYRN
GK³⁸¹
VB5006
Murine IL10 signal peptide (1-18),
murine IL10 (2-178), dimerization
unit (179-355), unit linker
(356-360), murine MOG 27-63
(361-397)
SEQ ID NO: 9
M¹PGSALLCCLLLLTGMRI¹⁸SRGQYSREDNNCTHFPVGQSHMLLELRTA
FSQVKTFFQTKDQLDNILLTDSLMQDFKGYLGCQALSEMIQFYLVEVMPQ
AEKHGPEIKEHLNSLGEKLKTLRMRLRRCHRFLPCENKSKAVEQVKSDFN
KLQDQGVYKAMNEFDIFINCIEAYMMIKMKS¹⁷⁸P¹⁷⁹SVIFLTKRGRQV
CADPSEEWVQKYVSDLELSAELKTPLGDTTHTEPKSCDTPPPCPRCPGGG
SSGGGSGGQPREPQVYTLPPSREEMTKNQVSLTCLVKGFYPSDIAVEWES
SGQPENNYNTTPPMLDSDGSFFLYSKLTVDKSRWQQGNIFSCSVMHEALH
NRFTQKSLSLSPGK³⁵⁵G³⁵⁶LGGL³⁶⁰SPGKNATGMEVGWYRSPFSRVV
HLYRNGKDQDAEAQP³⁹⁷
VB5009
Murine TGFβ1 signal peptide (1-29),
murine TGFβ1 30-390), dimerization
unit (391-567), unit linker
(568-572), murine MOG 35-55
(573-593)
SEQ ID NO: 10
MPPSGLRLLPLLLPLPWLLVLTPGRPAAGLSTCKTIDMELVKRKRIEAIR
GQILSKLRLASPPSQGEVPPGPLPEAVLALYNSTRDRVAGESADPEPEPE
ADYYAKEVTRVLMVDRNNAIYEKTKDISHSIYMFFNTSDIREAVPEPPLL
SRAELRLQRLKSSVEQHVELYQKYSNNSWRYLGNRLLTPTDTPEWLSFDV
TGVVRQWLNQGDGIQGFRFSAHCSCDSKDNKLHVEINGISPKRRGDLGTI
HDMNRPFLLLMATPLERAQHLHSSRHRRALDTNYCFSSTEKNCCVRQLYI
DFRKDLGWKWIHEPKGYHANFCLGPCPYIWSLDTQYSKVLALYNQHNPGA
SASPCCVPQALEPLPIVYYVGRKPKVEQLSNMIVRSCKCSPSVIFLTKRG
RQVCADPSEEWVQKYVSDLELSAELKTPLGDTTHTEPKSCDTPPPCPRCP
GGGSSGGGSGGQPREPQVYTLPPSREEMTKNQVSLTCLVKGFYPSDIAVE
WESSGQPENNYNTTPPMLDSDGSFFLYSKLTVDKSRWQQGNIFSCSVMHE
ALHNRFTQKSLSLSPGKGLGGLMEVGWYRSPFSRVVHLYRNGK
VB5017
Murine CTLA4 signal peptide (1-35),
murine CTLA4 (36-161), dimerization
unit (162-338), unit linker
(339-343), murine MOG 35-55
(344-364)
SEQ ID NO: 11
M¹ACLGLRRYKAQLQLPSRTWPFVALLTLLFIPVFS³⁵EAIQVTQPSVVL
ASSHGVASFPCEYSPSHNTDEVRVTVLRQTNDQMTEVCATTFTEKNTVGF
LDYPFCSGTFNESRVNLTIQGLRAVDTGLYLCKVELMYPPPYFVGMGNGT
QIYVIDPEPCPDSD¹⁶¹P¹⁶²SVIFLTKRGRQVCADPSEEWVQKYVSDLE
LSAELKTPLGDTTHTEPKSCDTPPPCPRCPGGGSSGGGSGGQPREPQVYT
LPPSREEMTKNQVSLTCLVKGFYPSDIAVEWESSGQPENNYNTTPPMLDS
DGSFFLYSKLTVDKSRWQQGNIFSCSVMHEALHNRFTQKSLSLSPGK³³⁸
G³³⁹LGGL³⁴³M³⁴⁴EVGWYRSPFSRVVHLYRNGK³⁶⁴
VB5022
Murine IgVH signal peptide (1-19),
murine anti-DEC205 scFv (20-265),
dimerization unit (266-442), unit
linker (443-447), murine Pan b 1
epitope (448-468)
SEQ ID NO: 12
M¹NFGLRLIFLVLTLKGVQC¹⁹D²⁰IQMTQSPSFLSTSLGNSITITCHAS
QNIKGWLAWYQQKSGNAPQLLIYKASSLQSGVPSRFSGSGSGTDYIFTIS
NLQPEDIATYYCQHYQSFPWTFGGGTKLELKGGGGSGGGGSGGGGSEVKL
LESGGGLVQPGGSLRLSCAASGFTFNDFYMNWIRQPPGQAPEWLGVIRNK
GNGYTTEVNTSVKGRFTISRDNTQNILYLQMNSLRAEDTAIYYCARGGPY
YYSGDDAPYWGQGVMVTVSS²⁶⁵P²⁶⁶SVIFLTKRGRQVCADPSEEWVQK
YVSDLELSAELKTPLGDTTHTEPKSCDTPPPCPRCPGGGSSGGGSGGQPR
EPQVYTLPPSREEMTKNQVSLTCLVKGFYPSDIAVEWESSGQPENNYNTT
PPMLDSDGSFFLYSKLTVDKSRWQQGNIFSCSVMHEALHNRFTQKSLSLS
PGK⁴⁴²G⁴⁴³LGGL⁴⁴⁷A⁴⁴⁸YKEQIKTLTNKLKAAEARAE⁴⁶⁸

### Embodiments

1. A tolerance-inducing construct comprising:
   i) a polynucleotide comprising a nucleotide sequence encoding a targeting unit targeting or capable of targeting antigen-presenting cells (APCs), a multimerization unit, such as a dimerization unit, and an antigenic unit; or
   ii) a polypeptide encoded by the nucleic acid sequence as defined in
   (i); or
   iii) a multimeric protein, such as a dimeric protein, consisting of multiple polypeptides as defined in (ii), such as two polypeptides;
   wherein the antigenic unit comprises one or more T cell epitopes of a self-antigen, an allergen, an alloantigen or a xenoantigen.
2. The tolerance-inducing construct according to embodiment 1, wherein said construct comprises a multimerization unit.
3. The tolerance-inducing construct according to embodiment 1, wherein said construct comprises a dimerization unit and/or wherein said multimeric protein is a dimeric protein.
4. The tolerance-inducing construct according to any of the preceding embodiments, wherein said targeting unit is capable of delivering the construct to an antigen-presenting cell and interacts with surface molecules on the APCs, without activating the APCs.
5. The tolerance-inducing construct according to any of the preceding embodiments, wherein said targeting unit is capable of binding to surface receptors on the APCs.
6. The tolerance-inducing construct according to any of the preceding embodiments, wherein said targeting unit comprises or consists of a moiety that binds to a receptor selected from the group consisting of TGFβ receptor, such as TGFβR1, TGFβR2, or TGFβR3, IL10R, such as IL-10RA and IL10-RB, IL2R, IL4R, IL6R, IL11R and IL13R, IL27R, IL35R, IL37R, CCR7, CD11b, CD11c, CD103, CD14, CD36, CD205, CD109, VISTA, MARCO, MHCII, MHCII, CD83, SIGLEC, MGL, CD80, CD86, Clec9A, Clec12A, Clec12B, DCIR2, Langerin, MR, DC-Sign, Treml4, Dectin-1, PDL1, PDL2 and HVEM.
7. The tolerance-inducing construct according to any of the preceding embodiments, wherein said moiety is selected from a natural ligand, a synthetic ligand and an antibody or part thereof, such as a scFv.
8. The tolerance-inducing construct according to any of the preceding embodiments, wherein said moiety is an antibody or part thereof, such as a scFv, with specificity for said receptor, and wherein binding to said receptor results in the antigen being presented in an anti-inflammatory, tolerogenic manner.
9. The tolerance-inducing construct according to any of the preceding embodiments, wherein said moiety is a synthetic ligand with specificity for said receptor, and wherein binding to the receptor results in the antigen being presented in an anti-inflammatory, tolerogenic manner.
10. The tolerance-inducing construct according to any of the preceding embodiments, wherein said moiety is a natural ligand.
11. The tolerance-inducing construct according to any of the preceding embodiments, wherein said natural ligand is selected from the group consisting of TGFβ, such as TGFβ1, TGFβ2 or TGFβ3, IL-10, IL2, IL4, IL6, IL11, IL13, IL27, IL35, IL37, CCL19, CCL21, ICAM-1 (Intercellular Adhesion Molecule 1 also known as CD54), keratin, VSIG-3, SCGB3A2, CTLA-4, such as the extracellular domain of CTLA-4, PD-1, such as the extracellular domain of PD-1, and BTLA.
12. The tolerance-inducing construct according to any of the preceding embodiments, wherein said natural ligand is selected from the group consisting of TGFβ, IL-10, SCGB3A2, CTLA-4, such as the extracellular domain of CTLA-4,
13. The tolerance-inducing construct according to any of the preceding embodiments, wherein said natural ligand is the extracellular domain of BTLA.
14. The tolerance-inducing construct according to any of the preceding embodiments, wherein said targeting unit consists of or comprises IL-10 or TGFβ, preferably human IL-10 or human TGFβ.
15. The tolerance-inducing construct according to any of the preceding embodiments, wherein said targeting unit comprises or consists of an amino acid sequence having at least 80% sequence identity to that of human TGFβ, such as an amino acid sequence selected from SEQ ID NO: 205-207.
16. The tolerance-inducing construct according to any of the preceding embodiments, wherein said targeting unit comprises or consists of an amino acid sequence having at least 85% sequence identity to the amino acid sequence of human TGFβ, such as an amino acid sequence selected from SEQ ID NO: 205-207, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.
17. The tolerance-inducing construct according to any of the preceding embodiments, wherein said targeting unit comprises or consists of an amino acid sequence of human TGFβ, such as an amino acid sequence selected from SEQ ID NO: 205-207, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.
18. The tolerance-inducing construct according to any of the preceding embodiments, wherein said targeting unit comprises or consists of an amino acid sequence having at least 80% sequence identity to that of human IL-10, such as the amino acid sequence of SEQ ID NO: 211.
19. The tolerance-inducing construct according to any of the preceding embodiments, wherein said targeting unit comprises or consists of an amino acid sequence having at least 85% sequence identity to the amino acid sequence of human IL-10, such as the amino acid sequence of SEQ ID NO: 211, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.
20. The tolerance-inducing construct according to any of the preceding embodiments, wherein said targeting unit comprises or consists of an amino acid sequence of human IL-10, such as the amino acid sequence of SEQ ID NO: 211, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.
21. The tolerance-inducing construct according to any of the preceding embodiments, wherein said targeting unit is or comprises SCGB3A2 or VSIG-3, such as human VSIG-3 or human SCGB3A2.
22. The tolerance-inducing construct according to any of the preceding embodiments, wherein said targeting unit comprises or consists of an amino acid sequence having at least 80% sequence identity to that of human VSIG-3, such as the amino acid sequence of SEQ ID NO: 215.
23. The tolerance-inducing construct according to any of the preceding embodiments, wherein said targeting unit comprises or consists of an amino acid sequence having at least 85% sequence identity to the amino acid sequence of human VSIG-3, such as the amino acid sequence of SEQ ID NO: 215, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.
24. The tolerance-inducing construct according to any of the preceding embodiments, wherein said targeting unit comprises or consists of an amino acid sequence of human VSIG-3, such as the amino acid sequence of SEQ ID NO: 215, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.
25. The tolerance-inducing construct according to any of the preceding embodiments, wherein said targeting unit comprises or consists of an amino acid sequence having at least 80% sequence identity to that of human SCGB3A2, such as the amino acid sequence of SEQ ID NO: 213.
26. The tolerance-inducing construct according to any of the preceding embodiments, wherein said targeting unit comprises or consists of an amino acid sequence having at least 85% sequence identity to the amino acid sequence of human SCGB3A2, such as the amino acid sequence of SEQ ID NO: 213, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.
27. The tolerance-inducing construct according to any of the preceding embodiments, wherein said targeting unit comprises or consists of an amino acid sequence of human SCGB3A2, such as the amino acid sequence of SEQ ID NO: 213, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.
28. The tolerance-inducing construct according to any of the preceding embodiments, wherein said targeting unit consists of or comprises an antibody or part thereof with specificity for CD205.
29. The tolerance-inducing construct according to any of the preceding embodiments, wherein said targeting unit consists of or comprises a scFv with specificity for CD205, such as an anti-DEC205 scFv, such as a scFv comprising or consisting of SEQ ID NO: 49.
30. The tolerance-inducing construct according to any of the preceding embodiments, wherein the antigenic unit comprises one or more T cell epitopes of a self-antigen, such as one T cell epitope of a self-antigen or multiple T cell epitopes of a self-antigen.
31. The tolerance-inducing construct according to any of the preceding embodiments, wherein the multiple T cell epitopes are of the same self-antigen or are of multiple different self-antigens.
32. The tolerance-inducing construct according to any of the preceding embodiments, wherein the antigenic unit comprises one or more linkers separating the T cell epitopes.
33. The tolerance-inducing construct according to any of the preceding embodiments, wherein the antigenic unit comprises multiple T cell epitopes of a self-antigen, an allergen, an alloantigen or a xenoantigen, and wherein the T cell epitopes are separated by a linkers.
34. The tolerance-inducing construct according to any of the preceding embodiments, wherein all T cell epitopes except the terminal T cell epitopes are arranged in subunits, wherein each subunit comprises or consists of a T cell epitope and a linker.
35. The tolerance-inducing construct according to any of the preceding embodiments, wherein all T cell epitopes except the N-terminal T cell epitope are arranged in subunits, wherein each subunit comprises or consists of a T cell epitope and a linker.
36. The tolerance-inducing construct according to any of the preceding embodiments, wherein all T cell epitopes except the C-terminal T cell epitope are arranged in subunits, wherein each subunit comprises or consists of a T cell epitope and a linker.
37. The tolerance-inducing construct according to any of the preceding embodiments, wherein the antigenic unit comprises n antigens and n−1 subunits, wherein each subunit comprises a T cell epitope of a self-antigen, an allergen, an alloantigen or a xenoantigen, and a linker, and further comprises a terminal T cell epitope.
38. The tolerance-inducing construct according to any of the preceding embodiments, wherein n is an integer of from 1 to 50, such as 3 to 50 or 15 to 40 or 10 to 30 or 10 to 25 or 10 to 20 or 15 to 30 or 15 to 25 or 15 to 20.
39. The tolerance-inducing construct according to any of the preceding embodiments, wherein the linker is non-immunogenic.
40. The tolerance-inducing construct according to any of the preceding embodiments, wherein the linker is a rigid linker or a flexible linker.
41. The tolerance-inducing construct according to any of the preceding embodiments, wherein the antigenic unit comprises one or more T cell epitopes of myelin basic protein (MBP), such as one T cell epitope of MBP or multiple T cell epitopes of MBP, myelin oligodendrocyte glycoprotein (MOG), such as one T cell epitope of MOG or multiple T cell epitopes of MOG, or proteolipid protein (PLP), such as one T cell epitope of PLP or multiple T cell epitopes of PLP.
42. The tolerance-inducing construct according to any of the preceding embodiments, wherein the antigenic unit comprises one or multiple T cell epitopes of MOG, such as one or multiple T cell epitopes of MOG comprising or consisting of a sequence selected from the group consisting of SEQ ID NO: 180-182.
43. The tolerance-inducing construct according to any of the preceding embodiments, wherein the antigenic unit comprises one or more T cell epitopes of an allergen, such as one T cell epitope of an allergen or multiple T cell epitopes of an allergen.
44. The tolerance-inducing construct according to any of the preceding embodiments, wherein the multiple T cell epitopes are of the same allergen or of multiple different allergens.
45. The tolerance-inducing construct according to any of the preceding embodiments, wherein the antigenic unit comprises one or more T cell epitopes of Fel d1, such as one T cell epitope of Fel d1 or multiple T cell epitopes of Fel d1.
46. The tolerance-inducing construct according to any of the preceding embodiments, wherein the antigenic unit comprises one or more T cell epitopes of Fel d4 and/or Fel d7, such as one or multiple T cell epitopes of Fel d4 and/or one or multiple T cell epitopes of Fel d7.
47. The tolerance-inducing construct according to any of the preceding embodiments, wherein the antigenic unit comprises one or more T cell epitopes of an alloantigen/xenoantigen, such as one T cell epitope of an alloantigen/xenoantigen or multiple T cell epitopes of an alloantigen/xenoantigen.
48. The tolerance-inducing construct according to any of the preceding embodiments, wherein the multiple T cell epitopes are of the same alloantigen/xenoantigen or of multiple different alloantigen/xenoantigens.
49. The tolerance-inducing construct according to any of the preceding embodiments, wherein the one or T cell epitopes have a length of from 7 to about 200 amino acids.
50. The tolerance-inducing construct according to any of the preceding embodiments, wherein the one or T cell epitopes have a length of from 7 to 150 amino acids, preferably of from 7 to 100 amino acids, such as from 9 to 100 amino acids or from 15 to 100 amino acids or from 9 to 60 amino acids or from 9 to 30 amino acids or from 15 to 60 of from 15 to 30 or from 20 to 75 amino acids or from 25 to 50 amino acids.
51. The tolerance-inducing construct according to any of the preceding embodiments, wherein the one or T cell epitopes have a length of 25, 26, 27, 28, 29, 30, 31, 32, 33, 34, 35, 36, 37, 38, 39, 40, 41, 42, 43, 44, 45, 46, 47, 48, 49, or 50 amino acids.
52. The tolerance-inducing construct according to any of the preceding embodiments, wherein the antigenic unit comprises one or more T cell epitopes having a length of from 7 to 11 amino acids.
53. The tolerance-inducing construct according to any of the preceding embodiments, wherein the antigenic unit comprises one or more T cell epitopes having a length of from 9 to 60 amino acids, such as from 9 to 30 amino acids, such as 15 to 60 amino acids, such as 15 to 30 amino acids.
54. The tolerance-inducing construct according to any of the preceding embodiments, wherein the antigenic unit comprises one or more T cell epitopes having a length a length of 15 amino acids.
55. The tolerance-inducing construct according to any of the preceding embodiments, wherein the antigenic unit comprises up to 3500 amino acids, such as from 60 to 3500 amino acids, such as from about 80 or about 100 or about 150 amino acids to about a 3000 amino acids, such as from about 200 to about 2500 amino acids, such as from about 300 to about 2000 amino acids or from about 400 to about 1500 amino acids or from about 500 to about 1000 amino acids.
56. The tolerance-inducing construct according to any of the preceding embodiments, wherein the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.
57. The tolerance-inducing construct according to any of the preceding embodiments, wherein the antigenic unit comprises 1 to 3 T cell epitopes, such as 1, 2, 3, or 1 to 5 T cell epitopes, such as 1, 2, 3, 4, 5, or 3 to 6 T cell epitopes, such as 3, 4, 5, 6, or 5 to 15 T cell epitopes, such as 5, 6, 7, 8, 9, 10, 11, 12, 13, 14, or 15 T cell epitopes, or 7 to 17 T cell epitopes, such as 7, 8, 9, 10, 11, 12, 13, 14, 15, 16, or 17 T cell epitopes, or 9 to 19 T cell epitopes, such as 9, 10, 11, 12, 13, 14, 15, 16, 17, 18, or 19 T cell epitopes.
58. The tolerance-inducing construct according to any of the preceding embodiments, wherein the one or more T cell epitopes are randomly arranged in the antigenic unit.
59. The tolerance-inducing construct according to any of the preceding embodiments, wherein the one or more T cell epitopes are arranged in the order of more antigenic to less antigenic in the direction from the multimerization unit to the end of the antigenic unit.
60. The tolerance-inducing construct according to any of the preceding embodiments, wherein the most hydrophobic T cell epitope(s) is/are positioned substantially in the middle of the antigenic unit and the most hydrophilic T cell epitope(s) is/are positioned closest to the multimerization unit or the end of the antigenic unit.
61. The tolerance-inducing construct according to any of the preceding embodiments, wherein the T cell epitopes are arranged in the order of more antigenic to less antigenic in the direction from dimerization unit to the end of the antigenic unit.
62. The tolerance-inducing construct according to any of the preceding embodiments, wherein the most hydrophobic T cell epitope(s) is/are positioned substantially in the middle of the antigenic unit and the most hydrophilic T cell epitope(s) is/are positioned closest to the dimerization unit or the end of the antigenic unit.
63. The tolerance-inducing construct according to any of the preceding embodiments, wherein the one or more T cell epitopes are arranged alternating between a hydrophilic and a hydrophobic T cell epitope.
64. The tolerance-inducing construct according to any of the preceding embodiments, wherein GC rich T cell epitopes are arranged such that there is at least one non-GC rich T cell epitope between them.
65. The tolerance-inducing construct according to any of the preceding embodiments, wherein multiple T cell epitopes are separated by T cell epitope linkers.
66. The tolerance-inducing construct according to any of the preceding embodiments, wherein the antigenic unit comprises n T cell epitopes and n−1 T cell epitope linkers.
67. The tolerance-inducing construct according to any of the preceding embodiments, wherein the T cell epitope linker is non-immunogenic.
68. The tolerance-inducing construct according to any of the preceding embodiments, wherein the T cell epitope linker is a flexible linker.
69. The tolerance-inducing construct according to any of the preceding embodiments, wherein the T cell epitope linker is a peptide consisting of from 4 to 20 amino acids, such as from 5 to 20 amino acids or 5 to 15 amino acids or 8 to 20 amino acids or 8 to 15 amino acids, such as 8, 9, 10, 11, 12, 13, 14, or 15 amino acids 10 to 15 amino acids or 8 to 12 amino acids, such as 8, 9, 10, 11, or 12 amino acids.
70. The tolerance-inducing construct according to any of the preceding embodiments, wherein the T cell epitope linker consists of 10 amino acids.
71. The tolerance-inducing construct according to any of the preceding embodiments, wherein all T cell epitope linkers comprised in the antigenic unit are preferably identical.
72. The tolerance-inducing construct according to any of the preceding embodiments, wherein the T cell epitope linker is a serine (S) and/or glycine (G) rich linker.
73. The tolerance-inducing construct according to any of the preceding embodiments, wherein the serine and/or glycine rich T cell epitope linker further comprises at least one leucine (L) residue, such as at least 1 or at least 2 or at least 3 or at least 4 leucine residues.
74. The tolerance-inducing construct according to any of the preceding embodiments, wherein the T cell epitope linker is serine-glycine linker having a length of 10 amino acids and comprises 1 or 2 leucine residues.
75. The tolerance-inducing construct according to any of the preceding embodiments, wherein the T cell epitope linker is a GSAT linker or SEG linker.
76. The tolerance-inducing construct according to any of the preceding embodiments, wherein the T cell epitope linker comprises or consists of GLGGL (SEQ ID NO: 90).
77. The tolerance-inducing construct according to any of the preceding embodiments, wherein the T cell epitope linker is a cleavable linker.
78. The tolerance-inducing construct according to any of the preceding embodiments, wherein the allergen is a food allergen, such as a shellfish allergen, such as tropomyosin, Arginin kinase, myosin light chain, sarcoplasmic calcium binding protein, troponin C, Triose-phosphate isomerase or actin.
79. The tolerance-inducing construct according to any of the preceding embodiments, wherein the allergen is Pan b 1, and optionally wherein the antigenic unit consists of or comprises a Pan b 1 T cell epitope (251-270).
80. The tolerance-inducing construct according to any of the preceding embodiments, wherein a cow's milk allergen, such as Bos d 4, Bos d 5, Bos d 6, Bos d 7, Bos d 8, Bos d 9, Bos d 10, Bos d 11 or Bos d 12.
81. The tolerance-inducing construct according to any of the preceding embodiments, wherein the allergen is an egg allergen, such as ovomucoid, ovalbumin, ovotransferin, conalbumin, Gal 3 3, egg lyaozyme or ovomucin.
82. The tolerance-inducing construct according to any of the preceding embodiments, wherein the allergen is OVA (257-264) comprising amino acid sequence SIINFEKL (SEQ ID NO: 45).
83. The tolerance-inducing construct according to any of the preceding embodiments, wherein the antigenic unit comprises the T cell epitope OVA (257-264).
84. The tolerance-inducing construct according to any of the preceding embodiments, wherein the allergen is a fish allergen, such as parvalbumin, enolase, aldolase or vitellogenin.
85. The tolerance-inducing construct according to any of the preceding embodiments, wherein the allergen is a fruit allergen, such as pathgenesis related protein 10, profilin, nsLTP, thaumatin-like protein, gibberellin regulated protein, isoflavone reductase related protein, class 1 chitinase, beta 1,3 glucanase, germin like protein, alkaline serine protease, pathogenesis-related protein 1, actinidin, phytocyctatin, kiwellin, major latex protein, cupin or 2S albumin.
86. The tolerance-inducing construct according to any of the preceding embodiments, wherein the allergen is a vegetable allergen, such as pathgenesis related protein 10, profilin, nsLTP type 1, nsLTP type protein 2, osmotin-like protein, isoflavone reductase-like protein, beta-fructofuranosidase, PR protein TSI-1, cyclophilin or FAD containing oxidase.
87. The tolerance-inducing construct according to any of the preceding embodiments, wherein the allergen is a wheat allergen, such as Tri a 12, Tri a 14, Tri a 15, Tri a 18, Tri a 19, Tri a 20, Tri a 21, Tri a 25, Tri a 26, Tri a 27, Tri a 28, Tri a 29, Tri a 30, Tri a 31, Tri a 32, Tri a 33, Tri a 34, Tri a 35, Tri a 36, Tri a 37 or Tri a 38.
88. The tolerance-inducing construct according to any of the preceding embodiments, wherein the allergen is a soy allergen, such as Gly m 1, Gly m 2, Gly m 3, Gly m 4, Gly m 5, Gly m 6, Gly m 7 or Gly m 8, Gly m agglutinin, Gly m Bd28K, Gly m 30 kD, Gly m CPI or Gly m TI.
89. The tolerance-inducing construct according to any of the preceding embodiments, wherein the allergen is a peanut allergen, such as Ara h 1, Ara h 2, Ara h 3, Ara h 5, Ara h 6, Ara h 7, Ara h 8, Ara h 9, Ara h 10, Ara h 11, Ara h 12, Ara h 13, Ara h 14, Ara h 15, Ara h 16, or Ara h 17.
90. The tolerance-inducing construct according to any of the preceding embodiments, wherein the allergen is a tree nut or seed allergen.
91. The tolerance-inducing construct according to any of the preceding embodiments, wherein the allergen is 11 S globulin, 7S globulin, 2S globulin, PR10, PR-14 nsLTP, Oleosin or profilin.
92. The tolerance-inducing construct according to any of the preceding embodiments, wherein the food allergen is buckwheat, celery, a color additive, garlic, gluten, oats, legumes, maize, mustard, poultry, meat, rice or sesame.
93. The tolerance-inducing construct according to any of the preceding embodiments, wherein the allergen is a bee venom allergen, such as Phospholipase A2, Hyaluronidase, acid phosphatase, melittin, allergen C/DPP, CRP/lcarapin or vitellogenin.
94. The tolerance-inducing construct according to any of the preceding embodiments, wherein the allergen is a vespid allergen, such as Phospholipase A1, hyaluronidase, protease, antigen 5, DPP IV or vitellogenin.
95. The tolerance-inducing construct according to any of the preceding embodiments, wherein the allergen is a latex allergen, such as Hev b 1, Hev b 2, Hev b 3, Hev b 4, Hev b 5, Hev b 6, Hev b 7, Hev b 8, Hev b 9, Hev b 10, Hev b 11, Hev b 12, Hev b 13, Hev b 14 or Hev b 15.
96. The tolerance-inducing construct according to any of the preceding embodiments, wherein the allergen is a dust mite allergen, such as a house dust mite allergen or a storage dust allergen, such as Der p 1, Der p 2, Der p 3, Der p 4, Der p 5, Der p 7, Der p 8, Der p 10, Der p 11, Der p 21, Der p 23, Der f 1, Der f 2, Der f 3, Der f 7, Der f 8 Der f 10, Blot t 1, Blot t 2, Blot t 3, Blot t 4, Blot t 5, Blot t 8, Blot t 10, Blot t 12 or Blot t 21.
97. The tolerance-inducing construct according to any of the preceding embodiments, wherein the antigenic unit consists of or comprises the Der p 1 T cell epitope (111-139).
98. The tolerance-inducing construct according to any of the preceding embodiments, wherein the allergen is a cockroach allergen, such as Bla g 1, Bla g 2, Bla g 3, Bla g 4, Bla g 5, Bla g 6, Bla g 7, Bla g 8, Bla g 11, Per a 1, Per a 2, Per a 3, Per a 6, Per a 7, Per a 9 or Per a 10.
99. The tolerance-inducing construct according to any of the preceding embodiments, wherein the allergen is a mold allergen, such as an Aspergillus fumigatus allergen, such as Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 14, Asp f 15, Asp f 16, Asp f 17, Asp f 18, Asp f22, Asp f23, Asp f27, Asp f28, Asp f29 or Asp f 34.
100. The tolerance-inducing construct according to any of the preceding embodiments, wherein the allergen is a fungal allergen, such as a Malassezia allergen, such as Mala f 1, Mala f 2, Mala f 3, Mala f 4, Mala f 5, Mala f 6, Mala f 7, Mala f 8, Mala f 9, Mala f 10, Mala f 11, Mala f 12 or Mala f 13 or MGL_1204.
101. The tolerance-inducing construct according to any of the preceding embodiments, wherein the allergen is furry animal allergen, such as a dog allergen, such as Can f 1, Can f 2, Can f 3, Can f 4, Can f 5, or Can f 6, or the allergen is a horse allergen, such as Ecu c 1, Ecu c 2, Ecu c 3 or Ecu c 4, or the allergen is a cat allergen, such as Fel d 1, Fel d 2, Fel d 3, Fel d 4, Fel d 5, Fel d 6, Fel d 7, or Fel d 8, or the allergen is a laboratory animal allergen, such as Lipocalin, urinary prealbumin, secretoglobulin or serum albumin.
102. The tolerance-inducing construct according to any of the preceding embodiments, wherein the allergen is a pollen allergen, such as a grass pollen allergen, such as timothy grass, orchard grass, Kentucky bluegrass, perennial rye, sweet vernal grass, bahia grass, johnson grass, Bermuda grass allergen, Phl p 1, Phl p 2, Phl p 3, Phl p 4, Phl p 5, Phl p 6, Phl p 7, Phl p 11, Phl p 12 or Phl p 13.
103. The tolerance-inducing construct according to any of the preceding embodiments, wherein the allergen is a tree pollen allergen, such as an alder, birch, hornbeam, hazel, European hophornbeam, chestnut, European beech, white oak, ash, privet, olive, lilac, cypress or cedar pollen allergen, such as Aln g 1 or Aln g 4, Bet v 1, Bet v 2, Bet v 3, Bet v 4, Bet v 6 or Bet v 7, Car b 1, Cor a 1, Cor a 2, Cor a 6, Cor a 8, Cor a 9, Cora 10, Cora 11, Cora 12, Cora 13, Cor a 14, Ost c 1, Cas 1, Cas 5, Cas 8, or Cas 9, Fag s 1, Que a 1, Fra e 1, Lig v 1, Ole e 1, Ole e 2, 3 Ole e, 4, Ole e 5, Ole e 6, Ole e 7, Ole e 8, Ole e 9, Ole e 10, Ole e 11, or Ole e 12, Syr v 1, Cha o 1, Cha o 2, Cry j 1, Cry j 2, Cup s 1, Cup s 3, Jun a 1, Jun a 2, Jun a 3, Jun o 4, Jun v 1, Jun v 3, Pla a 1, Pla a 2 or Pla a 3 or Pla or 1, Pla or 2 or Pla or 3.
104. The tolerance-inducing construct according to any of the preceding embodiments, wherein the antigenic unit consists of or comprises the Bet v 1 T cell epitope (139-152).
105. The tolerance-inducing construct according to any of the preceding embodiments, wherein the allergen is a weed pollen allergen, such as a ragweed, mugwort, sunflower, feverfew, pellitory, English plantain, annual mercury, goosefoot, Russian thistle or amaranth pollen allergen, such as Amb a 1, Amb a 4, Amb a 6, Amb a 8, Amb a 9, Amb a 10, or Amb a 11, Art v 1, Art v 3, Art v 4, Art v 5, or Art v 6, Hel a 1 or Hel a 2, Par j 1, Par j 2, Par j 3 or Par j 4, Pla I 1, Mer a 1, Che a 1, Che a 2 or Che a 3, Sal k 1, Sal k 4 or Sal k 5 or Ama r 2.
106. The tolerance-inducing construct according to any of the preceding embodiments, wherein the allergen is selected form environmental allergens such as insects, cockroaches, house dust mites or mold.
107. The tolerance-inducing construct according to any of the preceding embodiments, wherein the allergen causes an allergic disease selected from allergic rhinitis, asthma, atopic dermatitis, allergic gastroenteropathy, contact dermatitis, drug allergy or combinations thereof.
108. The tolerance-inducing construct according to any of the preceding embodiments, wherein the allergen is comprised in a drug with unwanted immunogenicity.
109. The tolerance-inducing construct according to any of the preceding embodiments, wherein the allergen is Factor VIII.
110. The tolerance-inducing construct according to any of the preceding embodiments, wherein the allergen is insulin.
111. The tolerance-inducing construct according to any of the preceding embodiments, wherein the allergen is one or more monoclonal antibodies used for therapy.
112. The tolerance-inducing construct according to any of the preceding embodiments, wherein the construct comprises T cell epitopes comprised in a self-allergen that is involved the self-antigen is involved in an autoimmune disease.
113. The tolerance-inducing construct according to any of the preceding embodiments, wherein the self-antigen is involved in multiple sclerosis (MS).
114. The tolerance-inducing construct according to any of the preceding embodiments, wherein the self-antigen is myelin oligodendrocyte glycoprotein (MOG), MAG, MOBP, CNPase, S100beta, transaldolase, myelin basic protein (MBP), myelin proteolipid protein (PLP).
115. The tolerance-inducing construct according to any of the preceding embodiments, wherein the antigenic unit comprises one or more T cell epitopes selected from the group consisting of MOG (35-55), MOG (27-63), PLP (139-151), PLP (131-159), PLP (178-191), PLP (170-199), MBP (84-104) and MBP (76-112).
116. The tolerance-inducing construct according to any of the preceding embodiments, wherein the antigenic unit comprises one or more T cell epitopes selected from the group consisting of SEQ ID NO: 185-190 or 192-197.
117. The tolerance-inducing construct according to any of the preceding embodiments, wherein the self-antigen is involved in type 1 diabetes mellitus.
118. The tolerance-inducing construct according to any of the preceding embodiments, wherein the self-antigen is glutamic acid decarboxylase 65-kilodalton isoform (GAD65), insulin, IA-2 or ZnT8, IGRP, ChgA, IAPP, peripherin, tetraspanin-7, GRP78, Urocortin-3 or Insulin gene enhancer protein isl-1.
119. The tolerance-inducing construct according to any of the preceding embodiments, wherein the self-antigen is involved in celiac disease.
120. The tolerance-inducing construct according to any of the preceding embodiments, wherein the self-antigen is α-gliadin, γ-gliadin, ω-gliadin, low molecular weight glutenin, high molecular weight glutenin, hordein, secalin or avenin b.
121. The tolerance-inducing construct according to any of the preceding embodiments, wherein the antigenic unit comprises the T cell epitope α-gliadin (76-95).
122. The tolerance-inducing construct according to any of the preceding embodiments, wherein the self-antigen is involved in rheumatoid arthritis.
123. The tolerance-inducing construct according to any of the preceding embodiments, wherein the self-antigen is collagen, heat shock protein 60 (HSP60), Band 3, small nuclear ribonucleoprotein D1 (SmD1), acetylcholine receptor (AChR) or myelin protein zero (P0).
124. The tolerance-inducing construct according to any of the preceding embodiments, wherein the self-antigen is involved in chronic inflammatory demyelinating polyradiculoneuropathy (CIDP) and the self-antigen is neurofascin 155.
125. The tolerance-inducing construct according to any of the preceding embodiments, wherein the self-antigen is involved in Hashimoto's thyroiditis (HT) and the self-antigen is thyroid peroxidase and/or thyroglobulin.
126. The tolerance-inducing construct according to any of the preceding embodiments, wherein the self-antigen is involved in pemphigus foliaceus and the self-antigen is desmosome-associated glycoprotein.
127. The tolerance-inducing construct according to any of the preceding embodiments, wherein the self-antigen is involved in pemphigus vulgaris and the self-antigen is desmoglein 3.
128. The tolerance-inducing construct according to any of the preceding embodiments, wherein the self-antigen is involved in thyroid eye disease (TED) and the self-antigen is calcium binding protein (calsequestrin).
129. The tolerance-inducing construct according to any of the preceding embodiments, wherein the self-antigen is involved in Grave's disease and the self-antigen is thyroid stimulating hormone receptor.
130. The tolerance-inducing construct according to any of the preceding embodiments, wherein the self-antigen is involved in primary biliary cirrhosis (PBC) and the self-antigen is antimitochondrial antibodies (AMAs), antinuclear antibodies (ANA), Rim-like/membrane (RL/M) and/or multiple nuclear dot (MND).
131. The tolerance-inducing construct according to any of the preceding embodiments, wherein the self-antigen is involved in myasthenia gravis and the self-antigen is acetylcholine receptor.
132. The tolerance-inducing construct according to any of the preceding embodiments, wherein the self-antigen is involved in insulin-resistant diabetes and the self-antigen is insulin receptor.
133. The tolerance-inducing construct according to any of the preceding embodiments, wherein the self-antigen is involved in autoimmune hemolytic anemia and the self-antigen is erythrocytes.
134. The tolerance-inducing construct according to any of the preceding embodiments, wherein the self-antigen is involved in rheumatoid arthritis and the self-antigens are citrullinated, homocitrullinated proteins and the Fc portion of IgG.
135. The tolerance-inducing construct according to any of the preceding embodiments, wherein the antigenic unit and the multimerization unit are connected by a unit linker.
136. The tolerance-inducing construct according to any of the preceding embodiments, wherein the antigenic unit and the dimerization unit are connected by a unit linker.
137. The tolerance-inducing construct according to any of the preceding embodiments, wherein the unit linker comprises a restriction site.
138. The tolerance-inducing construct according to any of the preceding embodiments, wherein the unit linker is a GLGGL linker (SEQ ID NO: 90) or a GLSGL linker (SEQ ID NO: 163).
139. The tolerance-inducing construct according to any of the preceding embodiments, wherein the unit linker comprises or consists of GGGGS (SEQ ID NO: 53), GGGGSGGGGS (SEQ ID NO: 56), (GGGGS)m (SEQ ID NO: 164), EAAAK (SEQ ID NO: 144), (EAAAK)m (SEQ ID NO: 165), (EAAAK)mGS (SEQ ID NO: 166), or (EAAK)mGS (SEQ ID NO: 31), where m is an integer greater than or equal to 1, GPSRLEEELRRRLTEPG (SEQ ID NO: 167), AAY or HEYGAEALERAG (SEQ ID NO: 168).
140. The tolerance-inducing construct according to any of the preceding embodiments, wherein the construct comprises a multimerization unit.
141. The tolerance-inducing construct according to any of the preceding embodiments, wherein the construct comprises a dimerization unit.
142. The tolerance-inducing construct according to any of the preceding embodiments, wherein the multimerization unit is a trimerization unit or a tetramerization unit.
143. The tolerance-inducing construct according to any of the preceding embodiments, wherein the multimerization unit is a trimerization unit, such as a collagen-derived trimerization unit, such as a human collagen-derived trimerization domain, such as human collagen derived XVIII trimerization domain or human collagen XV trimerization domain.
144. The tolerance-inducing construct according to any of the preceding embodiments, wherein the multimerization unit is a trimerization unit that comprises or consists of the nucleotide sequence with SEQ ID NO: 42, or an amino acid sequence encoded by said nucleotide sequence.
145. The tolerance-inducing construct according to any of the preceding embodiments, wherein the multimerization unit is trimerization unit is the C-terminal domain of T4 fibritin.
146. The tolerance-inducing construct according to any of the preceding embodiments, wherein the multimerization unit is a trimerization unit that comprises or consists of the amino acid sequence with SEQ ID NO: 43, or a nucleotide sequence encoding said amino acid sequence.
147. The tolerance-inducing construct according to any of the preceding embodiments, wherein the multimerization unit is a tetramerization unit, such as a domain derived from p53.
148. The tolerance-inducing construct according to any of the preceding embodiments, wherein the multimerization unit is a tetramerization unit that comprises or consists of the nucleic acid sequence with SEQ ID NO: 44, or an amino acid sequence encoded by said nucleic acid sequence.
149. The tolerance-inducing construct according to any of the preceding embodiments, wherein the dimerization unit comprises a hinge region.
150. The tolerance-inducing construct according to any of the preceding embodiments, wherein the dimerization unit comprises a hinge region and another domain that facilitates dimerization.
151. The tolerance-inducing construct according to any of the preceding embodiments, wherein the dimerization unit comprises a hinge region, a dimerization unit linker and another domain that facilitates dimerization, wherein the dimerization unit linker connects the hinge region to the other domain that facilitates dimerization.
152. The tolerance-inducing construct according to any of the preceding embodiments, wherein the dimerization unit comprises a hinge region, a dimerization unit linker and another domain that facilitates dimerization, wherein the dimerization unit linker connects the hinge region to the other domain that facilitates dimerization.
153. The tolerance-inducing construct according to any of the preceding embodiments, wherein the dimerization unit linker is a glycine-serine rich linker, such as GGGSSGGGSG (SEQ ID NO: 139).
154. The tolerance-inducing construct according to any of the preceding embodiments, wherein the hinge region is Ig derived, such as derived from IgG, such as IgG1, IgG2 or IgG3.
155. The tolerance-inducing construct according to any of the preceding embodiments, wherein the hinge region is derived from IgM, and optionally comprises or consists of the nucleotide sequence with SEQ ID NO: 47 or an amino acid sequence encoded by said nucleic acid sequence.
156. The tolerance-inducing construct according to any of the preceding embodiments, wherein he hinge region has the ability to form one or more covalent bonds, such as one or more disulfide bridges.
157. The tolerance-inducing construct according to any of the preceding embodiments, wherein the dimerization unit comprises or consists of a hinge exon h1 and hinge exon h4 (human hinge region 1 and human hinge region 4) having an amino acid sequence having at least 80% sequence identity to the amino acid sequence 1-27 of SEQ ID NO: 1.
158. The tolerance-inducing construct according to any of the preceding embodiments, wherein the dimerization unit comprises or consists of a hinge exon h1 and hinge exon h4 with an amino acid sequence having at least 85% sequence identity to the amino acid sequence 1-27 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.
159. The tolerance-inducing construct according to any of the preceding embodiments, wherein the dimerization unit comprises or consists of a hinge exon h1 and hinge exon h4 with the amino acid sequence 1-27 of SEQ ID NO: 1, or a nucleotide sequence encoding the amino acid sequence.
160. The tolerance-inducing construct according to any of the preceding embodiments, wherein the dimerization unit comprises or consists of a hinge exon h1 and hinge exon h4 with the amino acid sequence 1-27 of SEQ ID NO: 1, except that at the most ten amino acids have been substituted, deleted or inserted, such as at the most nine amino acids, such as at the most eight amino acids, such as at the most seven amino acids, such as at the most six amino acids, such as at the most five amino acids, such as at the most four amino acids, such as at the most three amino acids, such as at the most two amino acids or such as at the most one amino acid.
161. The tolerance-inducing construct according to any of the preceding embodiments, wherein the dimerization unit comprises another domain that facilitates dimerization, such as an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof.
162. The tolerance-inducing construct according to any of the preceding embodiments, wherein the other domain that facilitates dimerization is a carboxyterminal C domain derived from IgG, such as a carboxyterminal C domain derived from IgG3.
163. The tolerance-inducing construct according to any of the preceding embodiments, wherein the dimerization unit comprises or consists of a carboxyterminal C domain derived from IgG3 with an amino acid sequence having at least 80% sequence identity to the amino acid sequence 39-144 of SEQ ID NO: 1, or a nucleotide sequence encoding the amino acid sequence.
164. The tolerance-inducing construct according to any of the preceding embodiments, wherein the dimerization unit comprises or consists of a carboxyterminal C domain derived from IgG3 with an amino acid sequence having at least 85% sequence identity to the amino acid sequence 39-144 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.
165. The tolerance-inducing construct according to any of the preceding embodiments, wherein the dimerization unit comprises or consists of a carboxyterminal C domain derived from IgG3 with the amino acid sequence 39-144 of SEQ ID NO: 1.
166. The tolerance-inducing construct according to any of the preceding embodiments, wherein the dimerization unit comprises or consists of the amino acid sequence 39-144 of SEQ ID NO: 1, except that at the most 16 amino acids have been substituted, deleted or inserted, such as at the most 15, 14, 13, 12, 11, 10, 9, 8, 7, 6, 5, 4, 3, 2, or 1 amino acid.
167. The tolerance-inducing construct according to any of the preceding embodiments, wherein the immunoglobulin domain has the ability to form dimers via noncovalent interactions, such as hydrophobic interactions.
168. The tolerance-inducing construct according to any of the preceding embodiments, wherein the dimerization unit comprises a CH3 domain and does not comprise a CH2 domain or wherein the dimerization unit comprises a CH2 domain and does not comprise a CH3 domain.
169. The tolerance-inducing construct according to any of the preceding embodiments, wherein the dimerization unit comprises a hinge exon h1, a hinge exon h4, a dimerization unit linker and a CH3 domain of human IgG3.
170. The tolerance-inducing construct according to any of the preceding embodiments, wherein the dimerization unit comprises a polypeptide consisting of hinge exon h1, hinge exon h4, a dimerization unit linker and a CH3 domain of human IgG3.
171. The tolerance-inducing construct according to any of the preceding embodiments, wherein the dimerization unit consists of a polypeptide consisting of hinge exon h1, hinge exon h4, a dimerization unit linker and a CH3 domain of human IgG3.
172. The tolerance-inducing construct according to any of the preceding embodiments, wherein the dimerization unit comprises an amino acid sequence having at least 80% sequence identity to the amino acid sequence of SEQ ID NO: 1.
173. The tolerance-inducing construct according to any of the preceding embodiments, wherein the dimerization unit comprises an amino acid sequence having at least 85% sequence identity to the amino acid sequence of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.
174. The tolerance-inducing construct according to any of the preceding embodiments, wherein the dimerization unit consists of an amino acid sequence having at least 80% sequence identity to the amino acid sequence of SEQ ID NO: 1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.
175. The tolerance-inducing construct according to any of the preceding embodiments, wherein the dimerization unit consists of the amino acid sequence of SEQ ID NO: 1, or a nucleotide sequence encoding the amino acid sequence.
176. The tolerance-inducing construct according to any of the preceding embodiments, wherein the dimerization unit comprises the amino acid sequence of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.
177. The tolerance-inducing construct according to any of the preceding embodiments, wherein the dimerization unit consists of the amino acid sequence of SEQ ID NO: 1, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.
178. The tolerance-inducing construct according to any of the preceding embodiments, wherein the dimerization unit linker is a glycine-serine rich linker, such as GGGSSGGGSG (SEQ ID NO: 139).
179. The tolerance-inducing construct according to any of the preceding embodiments, wherein the polynucleotide further comprises a nucleotide sequence encoding a signal peptide.
180. The tolerance-inducing construct according to any of the preceding embodiments, wherein the signal peptide is located at the N-terminal end of the targeting unit or the C-terminal end of the targeting unit.
181. The tolerance-inducing construct according to any of the preceding embodiments, wherein the signal peptide is a human Ig VH signal peptide or a signal peptide which is naturally present at the N-terminus of any of the targeting units described herein, such as human signal peptide of human IL-10 or a human signal peptide of human TGFβ.
182. The tolerance-inducing construct according to any of the preceding embodiments, wherein the polynucleotide comprises a nucleotide sequence encoding a human IL-10 signal peptide and preferably comprises a nucleotide sequence encoding a human IL-10 targeting unit.
183. The tolerance-inducing construct according to any of the preceding embodiments, wherein the polynucleotide comprises a nucleotide sequence encoding a human Ig VH signal peptide and preferably comprises a nucleotide sequence encoding a scFv, such as human anti-DEC205.
184. The tolerance-inducing construct according to any of the preceding embodiments, wherein the polynucleotide comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%, sequence identity to the amino acid sequence of SEQ ID NO: 6 OR SEQ ID NO: 48.
185. The tolerance-inducing construct according to any of the preceding embodiments, wherein the polynucleotide comprises a nucleotide sequence encoding a signal peptide that comprises the amino acid sequence of SEQ ID NO: 6 OR SEQ ID NO: 48.
186. The tolerance-inducing construct according to any of the preceding embodiments, wherein the polynucleotide comprises a nucleotide sequence encoding a signal peptide that consists of an amino acid sequence having at least 80%, preferably at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence of SEQ ID NO: 6 OR SEQ ID NO: 48.
187. The tolerance-inducing construct according to any of the preceding embodiments, wherein the polynucleotide comprises a nucleotide sequence encoding a signal peptide with the amino acid sequence of SEQ ID NO: 6 OR SEQ ID NO: 48.
188. The tolerance-inducing construct according to any of the preceding embodiments, wherein the polynucleotide comprises a nucleotide sequence encoding a signal peptide that comprises or consists of an amino acid sequence of SEQ ID NO: 6 OR SEQ ID NO: 48, except that at the most five amino acids have been substituted, deleted or inserted, such as at the most four amino acids, such as at the most three amino acids, such as at the most two amino acids or such as at the most one amino acid.
189. The tolerance-inducing construct according to any of the preceding embodiments, wherein the polynucleotide is a DNA sequence or an RNA sequence.
190. A polynucleotide as defined in any of the preceding embodiments.
191. A vector comprising the polynucleotide according to embodiment 190.
192. A host cell comprising the polynucleotide according to embodiment 190 and/or the vector according to embodiment 191.
193. A polypeptide encoded by the nucleotide sequence as defined in any of the embodiments 1 to 189.
194. A dimeric protein as defined in any of embodiments 1 to 189 consisting of two polypeptides.
195. A multimeric protein as defined in any of embodiments 1 to 189 consisting of two or more polypeptides.
196. A pharmaceutical composition comprising the tolerance-inducing construct according to any of embodiments 1 to 189 and a pharmaceutically acceptable carrier.
197. A pharmaceutical composition comprising the polynucleotide according to any of embodiments 1 to 189, the vector according to embodiment 191, the polypeptide according to embodiment 193, the dimeric protein according to embodiment 194 or the multimeric protein according to embodiment 195 and a pharmaceutically acceptable carrier.
198. The pharmaceutical composition according to any of embodiments 196-197 further comprising one or more pharmaceutically acceptable excipients and/or diluents.
199. The pharmaceutical composition according to any of embodiments 196-198, wherein the pharmaceutically acceptable carrier is selected from the group consisting of saline, buffered saline, PBS, dextrose, water, glycerol, ethanol, sterile isotonic aqueous buffers, and combinations thereof
200. A method for preparing the pharmaceutical composition according to any of embodiments 196-199 which comprises a multimeric protein, dimeric protein or polypeptide, wherein the method comprises the following steps:
   i) transfecting cells with the polynucleotide according to embodiment 190;
   ii) culturing the cells;
   iii) collecting and purifying the multimeric protein, the dimeric protein or the polypeptide expressed from the cells; and
   iv) mixing the dimeric protein or polypeptide obtained from step iii) with a pharmaceutically acceptable carrier.
201. A method for preparing the pharmaceutical composition according to any of embodiments 196-199, wherein the pharmaceutical composition comprises the polynucleotide according to embodiment 190, the method comprises the following steps:
   i) preparing the polynucleotide;
   ii) optionally cloning the polynucleotide into an expression vector; and
   iii) mixing the polynucleotide obtained from step i) or the vector obtained from step ii) with the pharmaceutically acceptable carrier.
202. A method for treating a subject suffering from a condition involving undesirable immune reactions, such an autoimmune disease, allergic disease or graft rejection or being in need of prevention thereof, the method comprising administering to the subject the pharmaceutical composition according to any of embodiments 196-199.
203. A pharmaceutical composition according to any of embodiments 196-199 for use in the treatment of a condition involving undesirable immune reactions, such an autoimmune disease, allergic disease or graft rejection.
204. Use of the pharmaceutical composition according to any of embodiments 196-199 for the treatment of a condition involving undesirable immune reactions, such an autoimmune disease, allergic disease or graft rejection.
205. Use of the pharmaceutical composition according to any of embodiments 196-199 for the manufacture of a medicament for treatment of a condition involving undesirable immune reactions, such an autoimmune disease, allergic disease or graft rejection.
206. Use of the pharmaceutical composition according to any of embodiments 196-199 for treating a subject having a condition involving undesirable immune reactions, such an autoimmune disease, allergic disease or graft rejection.
207. A medicament comprising the tolerance-inducing construct according to any of embodiments 1 to 189 for treatment of a condition involving undesirable immune reactions, such an autoimmune disease, allergic disease or graft rejection.
208. Use of a pharmaceutical composition comprising a pharmaceutically acceptable carrier and the tolerance-inducing construct according to any of embodiments 1 to 189 for the manufacture of a medicament for the treatment of a subject having a condition involving undesirable immune reactions, such an autoimmune disease, allergic disease or graft rejection.
209. Use of a pharmaceutical composition comprising a pharmaceutically acceptable carrier and the tolerance-inducing construct according to any of embodiments 1 to 189 for the treatment of a subject having a condition involving undesirable immune reactions, such an autoimmune disease, allergic disease or graft rejection.
210. A pharmaceutical composition comprising a pharmaceutically acceptable carrier and the tolerance-inducing construct according to any of embodiments 1 to 189 when used in the treatment of a condition involving undesirable immune reactions, such an autoimmune disease, allergic disease or graft rejection.
211. A method for improving tolerance to a self-antigen, an allergen, an alloantigen or a xenoantigen using the tolerance-inducing construct according to any of embodiments 1 to 189.
212. A method for improving tolerance to a self-antigen, an allergen, an alloantigen or a xenoantigen in a subject, the method comprising administering to the subject the tolerance-inducing construct according to any of embodiments 1 to 189 or the pharmaceutical composition according to any of embodiments 196-199.

1.-63. (canceled)

64. A tolerance-inducing construct comprising:
i) a polynucleotide comprising a nucleotide sequence encoding a targeting unit which targets antigen-presenting cells, a multimerization unit and an antigenic unit; or
ii) a polypeptide encoded by the nucleic acid sequence as defined in (i); or
iii) a multimeric protein consisting of multiple polypeptides as defined in (ii);
wherein the antigenic unit comprises one or more T cell epitopes of a self-antigen, an allergen, an alloantigen or a xenoantigen and wherein the targeting unit interacts with a surface molecule on the antigen-presenting cell, without activating the cell.

65. The tolerance-inducing construct according to claim 64, wherein said tolerance-inducing construct comprises a dimerization unit and wherein said multimeric protein is a dimeric protein.

66. The tolerance-inducing construct according to claim 64, wherein the targeting unit comprises or consists of a moiety that binds to a receptor selected from TGFβR1, TGFβR2, TGFβR3, IL10R, IL-10RA and IL10-RB, IL2R, IL4R, IL6R, IL11R and IL13R, IL27R, IL35R, IL37R, CCR7, CD11b, CD11c, CD103, CD14, CD36, CD205, CD109, VISTA, MARCO, MHCII, MHCII, CD83, SIGLEC, MGL, CD80, CD86, Clec9A, Clec12A, Clec12B, DCIR2, Langerin, MR, DC-Sign, Treml4, Dectin-1, PDL1, PDL2 or HVEM.

67. The tolerance-inducing construct according to claim 66, wherein the moiety is an antibody or part thereof, or a synthetic ligand or a natural ligand.

68. The tolerance-inducing construct according to claim 67, wherein the natural ligand is TGFβ, IL-10, IL2, IL4, IL6, IL11, IL13, IL27, IL35, IL37, CCL19, CCL21, ICAM-1, keratin, VSIG-3, SCGB3A2, CTLA-4, PD-1, or BTLA.

69. The tolerance-inducing construct according to claim 64, wherein the antigenic unit comprises a) one T cell epitope of one self-antigen or one allergen or one alloantigen or one xenoantigen or b) multiple T cell epitopes of one self-antigen or one allergen or one alloantigen or one xenoantigen or c) multiple T cell epitopes of multiple different self-antigens or multiple different allergens or multiple different alloantigens or multiple different xenoantigens.

70. The tolerance-inducing construct according to claim 64, wherein the antigenic unit comprises one or more T cell epitopes of an allergen selected from food allergen, bee venom allergen, latex allergen, dust mite allergen, cockroach allergen, mold allergen, fungal allergen, furry animal allergen, pollen allergen or allergen comprised in a drug.

71. The tolerance-inducing construct according to claim 64, wherein the antigenic unit comprises one or more T cell epitopes of a self-antigen selected from multiple sclerosis self-antigen, type 1 diabetes mellitus self-antigen, celiac disease self-antigen, rheumatoid arthritis self-antigen, chronic inflammatory demyelinating polyradiculoneuropathy self-antigen, Hashimoto's thyroiditis self-antigen, pemphigus foliaceus self-antigen, pemphigus vulgaris self-antigen, thyroid eye disease self-antigen, Grave's disease self-antigen, primary biliary cirrhosis the self-antigen, myasthenia gravis self-antigen, insulin-resistant diabetes self-antigen or hemolytic anemia self-antigen.

72. The tolerance-inducing construct according to claim 65, wherein the dimerization unit comprises hinge exon h1 and hinge exon h4, a dimerization unit linker and a CH3 domain of human IgG3.

73. The tolerance-inducing construct according to claim 64, wherein the tolerance-inducing construct is the polynucleotide of (i).

74. The tolerance-inducing construct according to claim 73, wherein said tolerance-inducing construct is comprised in a vector or a host cell.

75. The tolerance-inducing construct according to claim 64, wherein the tolerance-inducing construct is the polypeptide of (ii).

76. A dimeric protein consisting of two polypeptides according to claim 75, wherein said polypeptides comprise a dimerization unit.

77. A pharmaceutical composition comprising the tolerance-inducing construct according to claim 64 and a pharmaceutically acceptable carrier.

78. A method for improving tolerance to a self-antigen, an allergen, an alloantigen or a xenoantigen in a subject, the method comprising administering to the subject the tolerance-inducing construct according to claim 64.


SEQUENCE LISTING (220 sequences)

SEQ ID NO: 1 - protein, 144 aa, Artificial sequence
  Synthetic sequence
  MISC_FEATURE: Dimerization unit
ELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 2 - protein, 468 aa, Artificial sequence
  Synthetic sequence
  MISC_FEATURE: VB5003
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 3 - protein, 484 aa, Artificial sequence
  Synthetic sequence
  MISC_FEATURE: VB5004
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 4 - protein, 294 aa, Artificial sequence
  Synthetic sequence
  MISC_FEATURE: VB5012
MKLVSIFLLVTIGICGYSATALLINRLPVVDKLPVPLDDIIPSFDPLKMLLKTLGISVEHLVTGLKKCVDELGPEASEAVKKLLEALSHLVPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 5 - protein, 631 aa, Artificial sequence
  Synthetic sequence
  MISC_FEATURE: VB5013
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVGVIAGAVGTGAVLIVICLALISGAFFYWRSKNKEEEEEEIPNEIREDDLPPKCSSAKAFHTEISSSENNTLTSSNTYNSRYWNNNPKPHRNTESFNHFSDLRQSFSGNAVIPSIYANGNHLVLGPHKTLVVTANRGSSPQVLPRNNGSVSRKPWPQHTHSYTVSQMTLERIGAVPVMVPAQSRAGSLVPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 6 - protein, 19 aa, Artificial sequence
  Synthetic sequence
  MISC_FEATURE: Murine IgVH signal peptide (1-19)
MNFGLRLIFLVLTLKGVQC

SEQ ID NO: 7 - protein, 467 aa, Artificial sequence
  Synthetic sequence
  MISC_FEATURE: VB5016
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLTNMFTYEIAPVFVLLEYVTL

SEQ ID NO: 8 - protein, 381 aa, Artificial sequence
  Synthetic sequence
  MISC_FEATURE: VB5005
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 9 - protein, 397 aa, Artificial sequence
  Synthetic sequence
  MISC_FEATURE: VB5006
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 10 - protein, 593 aa, Artificial sequence
  Synthetic sequence
  MISC_FEATURE: VB5009
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 11 - protein, 364 aa, Artificial sequence
  Synthetic sequence
  MISC_FEATURE: VB5017
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 12 - protein, 468 aa, Artificial sequence
  Synthetic sequence
  MISC_FEATURE: VB5022
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLAYKEQIKTLTNKLKAAEARAE

SEQ ID NO: 13 - protein, 435 aa, Artificial sequence
  Synthetic sequence
  MISC_FEATURE: VB5003b
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 14 - protein, 348 aa, Artificial sequence
  Synthetic sequence
  MISC_FEATURE: VB5005b
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 15 - protein, 331 aa, Artificial sequence
  Synthetic sequence
  MISC_FEATURE: VB5017a
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMEVGWYRSPFSRVVHLYRNGK

SEQ ID NO: 16 - protein, 426 aa, Artificial sequence
  Synthetic sequence
  MISC_FEATURE: VB5046
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 17 - protein, 451 aa, Artificial sequence
  Synthetic sequence
  MISC_FEATURE: VB50048
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 18 - protein, 279 aa, Artificial sequence
  Synthetic sequence
  MISC_FEATURE: VB5052
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 19 - protein, 364 aa, Artificial sequence
  Synthetic sequence
  MISC_FEATURE: VB5058
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 20 - protein, 576 aa, Artificial sequence
  Synthetic sequence
  MISC_FEATURE: VB5059
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 21 - protein, 277 aa, Artificial sequence
  Synthetic sequence
  MISC_FEATURE: VB5060
MKLVSIFLLVTIGICGYSATALLINRLPVVDKLPVPLDDIIPSFDPLKMLLKTLGISVEHLVTGLKKCVDELGPEASEAVKKLLEALSHLVELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 22 - protein, 347 aa, Artificial sequence
  Synthetic sequence
  MISC_FEATURE: VB5061
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 23 - protein, 355 aa, Artificial sequence
  Synthetic sequence
  MISC_FEATURE: VB5071
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 24 - protein, 56 aa, Artificial sequence
  Synthetic sequence
  MISC_FEATURE: VB5051
MNFGLRLIFLVLTLKGVQCSPGKNATGMEVGWYRSPFSRVVHLYRNGKDQDAEQAP

SEQ ID NO: 25 - protein, 39 aa, Artificial sequence
  Synthetic sequence
  MISC_FEATURE: VB5014b
MNFGLRLIFLVLTLKGVQCTNMFTYEIAPVFVLLEYVTL

SEQ ID NO: 26 - protein, 262 aa, Artificial sequence
  Synthetic sequence
  MISC_FEATURE: VB5015b
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLTNMFTYEIAPVFVLLEYVTL

SEQ ID NO: 27 - protein, 434 aa, Artificial sequence
  Synthetic sequence
  MISC_FEATURE: VB5016b
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLTNMFTYEIAPVFVLLEYVTL

SEQ ID NO: 28 - DNA, 45 bp, Artificial sequence
  Synthetic sequence
  misc_feature: Dimerization unit
gagcccaaatcttgtgacacacctcccccgtgcccaaggtgccca

SEQ ID NO: 29 - protein, 171 aa, Artificial sequence
  Synthetic sequence
  MISC_FEATURE: Met e 1 antigenic unit
KEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHN

SEQ ID NO: 30 - protein, 274 aa, Artificial sequence
  Synthetic sequence
  MISC_FEATURE: Met e 1 antigenic unit
MKLEKDNAMDRADTLEQQNKEANNRAEKSEEEVFGLQKKLQQLENDLDSVQEALLKANQHLEEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 31 - protein, 6 aa, Artificial sequence
  Synthetic sequence
  MISC_FEATURE: The indicated sequence can be repeated m times, where m is an integer greater than or equal to 1
EAAKGS

SEQ ID NO: 32 - protein, 434 aa, Artificial sequence
  Synthetic sequence
  MISC_FEATURE: VB5024
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKEVDRLEDELVNEKEKYKSI

SEQ ID NO: 33 - protein, 153 aa, Artificial sequence
  Synthetic sequence
  MISC_FEATURE: Human IL2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 34 - protein, 413 aa, Artificial sequence
  Synthetic sequence
  MISC_FEATURE: VB5030
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHN

SEQ ID NO: 35 - protein, 516 aa, Artificial sequence
  Synthetic sequence
  MISC_FEATURE: VB5079
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMKLEKDNAMDRADTLEQQNKEANNRAEKSEEEVFGLQKKLQQLENDLDSVQEALLKANQHLEEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 36 - DNA, 462 bp, Artificial sequence
  Synthetic sequence
  misc_feature: Human IL2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 37 - protein, 56 aa, Artificial sequence
  Synthetic sequence
  MISC_FEATURE: VB5001b
MNFGLRLIFLVLTLKGVQCSPGKNATGMEVGWYRSPFSRVVHLYRNGKDQDAEAQP

SEQ ID NO: 38 - protein, 279 aa, Artificial sequence
  Synthetic sequence
  MISC_FEATURE: VB5002b
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 39 - protein, 451 aa, Artificial sequence
  Synthetic sequence
  MISC_FEATURE: VB5004b
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 40 - protein, 363 aa, Artificial sequence
  Synthetic sequence
  MISC_FEATURE: VB5006b
PGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 41 - protein, 277 aa, Artificial sequence
  Synthetic sequence
  MISC_FEATURE: VB5012b
MKLVSIFLLVTIGICGYSATALLINRLPVVDKLPVPLDDIIPSFDPLKMLLKTLGISVEHLVTGLKKCVDELGPEASEAVKKLLEALSHLVELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 42 - DNA, 180 bp, Artificial sequence
  Synthetic sequence
  misc_feature: Trimerization unit
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 43 - protein, 27 aa, Artificial sequence
  Synthetic sequence
  MISC_FEATURE: Trimerization unit
GYIPEAPRDGQAYVRKDGEWVLLSTFL

SEQ ID NO: 44 - DNA, 117 bp, Homo sapiens
  misc_feature: Tetramerization unit
aagcctctggacggagagtatttcactctccagatccggggccccgaaaggttcgaaatgttccgggagcttaacgaggccttggagctgaaagacgcacaggccggaaaggaaccg

SEQ ID NO: 45 - protein, 8 aa, Homo sapiens
  MISC_FEATURE: OVA (257-264)
SIINFEKL

SEQ ID NO: 46 - protein, 15 aa, Homo sapiens
  MISC_FEATURE: Dimerization unit
EPKSCDTPPPCPRCP

SEQ ID NO: 47 - DNA, 333 bp, Homo sapiens
  misc_feature: IgM hinge
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 48 - protein, 18 aa, Homo sapiens
  MISC_FEATURE: Murine Ig VH signal peptide
MNFGLRLIFLVLTLKGVQ

SEQ ID NO: 49 - protein, 246 aa, Homo sapiens
  MISC_FEATURE: Murine scFv anti-DEC205
DIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSS

SEQ ID NO: 50 - protein, 18 aa, Homo sapiens
  MISC_FEATURE: Natural leader sequence murine IL-10
MPGSALLCCLLLLTGMRI

SEQ ID NO: 51 - protein, 10 aa, Artificial sequence
  Synthetic sequence
  MISC_FEATURE: Linker
GGGGSGGGSS

SEQ ID NO: 52 - protein, 5 aa, Artificial sequence
  Synthetic sequence
  MISC_FEATURE: Linker
GGGSG

SEQ ID NO: 53 - protein, 5 aa, Artificial sequence
  Synthetic sequence
  MISC_FEATURE: Linker
GGGGS

SEQ ID NO: 54 - protein, 6 aa, Artificial sequence
  Synthetic sequence
  MISC_FEATURE: Linker
SGSSGS

SEQ ID NO: 55 - protein, 5 aa, Artificial sequence
  Synthetic sequence
  MISC_FEATURE: Linker
GGSGG

SEQ ID NO: 56 - protein, 10 aa, Artificial sequence
  Synthetic sequence
  MISC_FEATURE: Linker
GGGGSGGGGS

SEQ ID NO: 57 - protein, 15 aa, Artificial sequence
  Synthetic sequence
  MISC_FEATURE: Linker
GGGGSGGGGSGGGGS

SEQ ID NO: 58 - protein, 20 aa, Artificial sequence
  Synthetic sequence
  MISC_FEATURE: Linker
GGGGSGGGGSGGGGSGGGGS

SEQ ID NO: 59 - protein, 25 aa, Artificial sequence
  Synthetic sequence
  MISC_FEATURE: Linker
GGGGSGGGGSGGGGSGGGGSGGGGS

SEQ ID NO: 60 - protein, 5 aa, Artificial sequence
  Synthetic sequence
  MISC_FEATURE: Linker
GGGSS

SEQ ID NO: 61 - protein, 10 aa, Artificial sequence
  Synthetic sequence
  MISC_FEATURE: Linker
GGGSSGGGSS

SEQ ID NO: 62 - protein, 15 aa, Artificial sequence
  Synthetic sequence
  MISC_FEATURE: Linker
GGGSSGGGSSGGGSS

SEQ ID NO: 63 - protein, 20 aa, Artificial sequence
  Synthetic sequence
  MISC_FEATURE: Linker
GGGSSGGGSSGGGSSGGGSS

SEQ ID NO: 64 - protein, 25 aa, Artificial sequence
  Synthetic sequence
  MISC_FEATURE: Linker
GGGSSGGGSSGGGSSGGGSSGGGSS

SEQ ID NO: 65 - protein, 10 aa, Artificial sequence
  Synthetic sequence
  MISC_FEATURE: Linker
GGGSGGGGSG

SEQ ID NO: 66 - protein, 15 aa, Artificial sequence
  Synthetic sequence
  MISC_FEATURE: Linker
GGGSGGGGSGGGGSG

SEQ ID NO: 67 - protein, 20 aa, Artificial sequence
  Synthetic sequence
  MISC_FEATURE: Linker
GGGSGGGGSGGGGSGGGGSG

SEQ ID NO: 68 - protein, 25 aa, Artificial sequence
  Synthetic sequence
  MISC_FEATURE: Linker
GGGSGGGGSGGGGSGGGGSGGGGSG

SEQ ID NO: 69 - protein, 12 aa, Artificial sequence
  Synthetic sequence
  MISC_FEATURE: Linker
SGSSGSSGSSGS

SEQ ID NO: 70 - protein, 18 aa, Artificial sequence
  Synthetic sequence
  MISC_FEATURE: Linker
SGSSGSSGSSGSSGSSGS

SEQ ID NO: 71 - protein, 24 aa, Artificial sequence
  Synthetic sequence
  MISC_FEATURE: Linker
SGSSGSSGSSGSSGSSGSSGSSGS

SEQ ID NO: 72 - protein, 30 aa, Artificial sequence
  Synthetic sequence
  MISC_FEATURE: Linker
SGSSGSSGSSGSSGSSGSSGSSGSSGSSGS

SEQ ID NO: 73 - protein, 5 aa, Artificial sequence
  Synthetic sequence
  MISC_FEATURE: Linker
LGGGS

SEQ ID NO: 74 - protein, 5 aa, Artificial sequence
  Synthetic sequence
  MISC_FEATURE: Linker
GLGGS

SEQ ID NO: 75 - protein, 5 aa, Artificial sequence
  Synthetic sequence
  MISC_FEATURE: Linker
GGLGS

SEQ ID NO: 76 - protein, 5 aa, Artificial sequence
  Synthetic sequence
  MISC_FEATURE: Linker
GGGLS

SEQ ID NO: 77 - protein, 5 aa, Artificial sequence
  Synthetic sequence
  MISC_FEATURE: Linker
GGGGL

SEQ ID NO: 78 - protein, 5 aa, Artificial sequence
  Synthetic sequence
  MISC_FEATURE: Linker
LGGSG

SEQ ID NO: 79 - protein, 5 aa, Artificial sequence
  Synthetic sequence
  MISC_FEATURE: Linker
GLGSG

SEQ ID NO: 80 - protein, 5 aa, Artificial sequence
  Synthetic sequence
  MISC_FEATURE: Linker
GGLSG

SEQ ID NO: 81 - protein, 5 aa, Artificial sequence
  Synthetic sequence
  MISC_FEATURE: Linker
GGGLG

SEQ ID NO: 82 - protein, 5 aa, Artificial sequence
  Synthetic sequence
  MISC_FEATURE: Linker
GGGSL

SEQ ID NO: 83 - protein, 5 aa, Artificial sequence
  Synthetic sequence
  MISC_FEATURE: Linker
LGGSS

SEQ ID NO: 84 - protein, 5 aa, Artificial sequence
  Synthetic sequence
  MISC_FEATURE: Linker
GLGSS

SEQ ID NO: 85 - protein, 5 aa, Artificial sequence
  Synthetic sequence
  MISC_FEATURE: Linker
GGLSS

SEQ ID NO: 86 - protein, 5 aa, Artificial sequence
  Synthetic sequence
  MISC_FEATURE: Linker
LGLGS

SEQ ID NO: 87 - protein, 5 aa, Artificial sequence
  Synthetic sequence
  MISC_FEATURE: Linker
GLGLS

SEQ ID NO: 88 - protein, 5 aa, Artificial sequence
  Synthetic sequence
  MISC_FEATURE: Linker
GLLGS

SEQ ID NO: 89 - protein, 5 aa, Artificial sequence
  Synthetic sequence
  MISC_FEATURE: Linker
LGGLS

SEQ ID NO: 90 - protein, 5 aa, Artificial sequence
  Synthetic sequence
  MISC_FEATURE: Linker
GLGGL

SEQ ID NO: 91 - protein, 10 aa, Artificial sequence
  Synthetic sequence
  MISC_FEATURE: Linker
GLGGLGLGGL

SEQ ID NO: 92 - protein, 15 aa, Artificial sequence
  Synthetic sequence
  MISC_FEATURE: Linker
GLGGLGLGGLGLGGL

SEQ ID NO: 93 - protein, 20 aa, Artificial sequence
  Synthetic sequence
  MISC_FEATURE: Linker
GLGGLGLGGLGLGGLGLGGL

SEQ ID NO: 94 - protein, 25 aa, Artificial sequence
  Synthetic sequence
  MISC_FEATURE: Linker
GLGGLGLGGLGLGGLGLGGLGLGGL

SEQ ID NO: 95 - protein, 5 aa, Artificial sequence
  Synthetic sequence
  MISC_FEATURE: Linker
LGLSG

SEQ ID NO: 96 - protein, 5 aa, Artificial sequence
  Synthetic sequence
  MISC_FEATURE: Linker
GLLSG

SEQ ID NO: 97 - protein, 5 aa, Artificial sequence
  Synthetic sequence
  MISC_FEATURE: Linker
GGLSL

SEQ ID NO: 98 - protein, 5 aa, Artificial sequence
  Synthetic sequence
  MISC_FEATURE: Linker
GGLLG

SEQ ID NO: 99 - protein, 5 aa, Artificial sequence
  Synthetic sequence
  MISC_FEATURE: Linker
GLGSL

SEQ ID NO: 100 - protein, 5 aa, Artificial sequence
  Synthetic sequence
  MISC_FEATURE: Linker
LGLSS

SEQ ID NO: 101 - protein, 5 aa, Artificial sequence
  Synthetic sequence
  MISC_FEATURE: Linker
GGLLS

SEQ ID NO: 102 - protein, 10 aa, Artificial sequence
  Synthetic sequence
  MISC_FEATURE: Linker
LGGGSGGGGS

SEQ ID NO: 103 - protein, 10 aa, Artificial sequence
  Synthetic sequence
  MISC_FEATURE: Linker
GLGGSGGGGS

SEQ ID NO: 104 - protein, 10 aa, Artificial sequence
  Synthetic sequence
  MISC_FEATURE: Linker
GGLGSGGGGS

SEQ ID NO: 105 - protein, 10 aa, Artificial sequence
  Synthetic sequence
  MISC_FEATURE: Linker
GGGLSGGGGS

SEQ ID NO: 106 - protein, 10 aa, Artificial sequence
  Synthetic sequence
  MISC_FEATURE: Linker
GGGGLGGGGS

SEQ ID NO: 107 - protein, 10 aa, Artificial sequence
  Synthetic sequence
  MISC_FEATURE: Linker
LGGSGGGGSG

SEQ ID NO: 108 - protein, 10 aa, Artificial sequence
  Synthetic sequence
  MISC_FEATURE: Linker
GLGSGGGGSG

SEQ ID NO: 109 - protein, 10 aa, Artificial sequence
  Synthetic sequence
  MISC_FEATURE: Linker
GGLSGGGGSG

SEQ ID NO: 110 - protein, 10 aa, Artificial sequence
  Synthetic sequence
  MISC_FEATURE: Linker
GGGLGGGGSG

SEQ ID NO: 111 - protein, 10 aa, Artificial sequence
  Synthetic sequence
  MISC_FEATURE: Linker
GGGSLGGGSG

SEQ ID NO: 112 - protein, 10 aa, Artificial sequence
  Synthetic sequence
  MISC_FEATURE: Linker
LGGSSGGGSS

SEQ ID NO: 113 - protein, 10 aa, Artificial sequence
  Synthetic sequence
  MISC_FEATURE: Linker
GLGSSGGGSS

SEQ ID NO: 114 - protein, 10 aa, Artificial sequence
  Synthetic sequence
  MISC_FEATURE: Linker
GGLSSGGGSS

SEQ ID NO: 115 - protein, 10 aa, Artificial sequence
  Synthetic sequence
  MISC_FEATURE: Linker
GGGLSGGGSS

SEQ ID NO: 116 - protein, 10 aa, Artificial sequence
  Synthetic sequence
  MISC_FEATURE: Linker
GGGSLGGGSS

SEQ ID NO: 117 - protein, 10 aa, Artificial sequence
  Synthetic sequence
  MISC_FEATURE: Linker
LGGGSLGGGS

SEQ ID NO: 118 - protein, 10 aa, Artificial sequence
  Synthetic sequence
  MISC_FEATURE: Linker
GLGGSGLGGS

SEQ ID NO: 119 - protein, 10 aa, Artificial sequence
  Synthetic sequence
  MISC_FEATURE: Linker
GGLGSGGLGS

SEQ ID NO: 120 - protein, 10 aa, Artificial sequence
  Synthetic sequence
  MISC_FEATURE: Linker
GGGLSGGGLS

SEQ ID NO: 121 - protein, 10 aa, Artificial sequence
  Synthetic sequence
  MISC_FEATURE: Linker
GGGGLGGGGL

SEQ ID NO: 122 - protein, 10 aa, Artificial sequence
  Synthetic sequence
  MISC_FEATURE: Linker
LGGSGLGGSG

SEQ ID NO: 123 - protein, 10 aa, Artificial sequence
  Synthetic sequence
  MISC_FEATURE: Linker
GLGSGGLGSG

SEQ ID NO: 124 - protein, 10 aa, Artificial sequence
  Synthetic sequence
  MISC_FEATURE: Linker
GGLSGGGLSG

SEQ ID NO: 125 - protein, 10 aa, Artificial sequence
  Synthetic sequence
  MISC_FEATURE: Linker
GGGLGGGGLG

SEQ ID NO: 126 - protein, 10 aa, Artificial sequence
  Synthetic sequence
  MISC_FEATURE: Linker
GGGSLGGGSL

SEQ ID NO: 127 - protein, 10 aa, Artificial sequence
  Synthetic sequence
  MISC_FEATURE: Linker
LGGSSLGGSS

SEQ ID NO: 128 - protein, 10 aa, Artificial sequence
  Synthetic sequence
  MISC_FEATURE: Linker
GLGSSGLGSS

SEQ ID NO: 129 - protein, 10 aa, Artificial sequence
  Synthetic sequence
  MISC_FEATURE: Linker
GGLSSGGLSS

SEQ ID NO: 130 - protein, 6 aa, Artificial sequence
  Synthetic sequence
  MISC_FEATURE: Linker
GSGGGA

SEQ ID NO: 131 - protein, 12 aa, Artificial sequence
  Synthetic sequence
  MISC_FEATURE: Linker
GSGGGAGSGGGA

SEQ ID NO: 132 - protein, 18 aa, Artificial sequence
  Synthetic sequence
  MISC_FEATURE: Linker
GSGGGAGSGGGAGSGGGA

SEQ ID NO: 133 - protein, 24 aa, Artificial sequence
  Synthetic sequence
  MISC_FEATURE: Linker
GSGGGAGSGGGAGSGGGAGSGGGA

SEQ ID NO: 134 - protein, 10 aa, Artificial sequence
  Synthetic sequence
  MISC_FEATURE: Linker
GENLYFQSGG

SEQ ID NO: 135 - protein, 10 aa, Artificial sequence
  Synthetic sequence
  MISC_FEATURE: Linker
SGGGSSGGGS

SEQ ID NO: 136 - protein, 10 aa, Artificial sequence
  Synthetic sequence
  MISC_FEATURE: Linker
SSGGGSSGGG

SEQ ID NO: 137 - protein, 10 aa, Artificial sequence
  Synthetic sequence
  MISC_FEATURE: Linker
GGSGGGGSGG

SEQ ID NO: 138 - protein, 10 aa, Artificial sequence
  Synthetic sequence
  MISC_FEATURE: Linker
GSGSGSGSGS

SEQ ID NO: 139 - protein, 10 aa, Artificial sequence
  Synthetic sequence
  MISC_FEATURE: Linker
GGGSSGGGSG

SEQ ID NO: 140 - protein, 6 aa, Artificial sequence
  Synthetic sequence
  MISC_FEATURE: Linker
GGGSSS

SEQ ID NO: 141 - protein, 8 aa, Artificial sequence
  Synthetic sequence
  MISC_FEATURE: Linker
GLGGLAAA

SEQ ID NO: 142 - protein, 12 aa, Artificial sequence
  Synthetic sequence
  MISC_FEATURE: Linker
KPEPKPAPAPKP

SEQ ID NO: 143 - protein, 12 aa, Artificial sequence
  Synthetic sequence
  MISC_FEATURE: Linker
AEAAAKEAAAKA

SEQ ID NO: 144 - protein, 5 aa, Artificial sequence
  Synthetic sequence
  MISC_FEATURE: Linker
EAAAK

SEQ ID NO: 145 - protein, 10 aa, Artificial sequence
  Synthetic sequence
  MISC_FEATURE: Linker
EAAAKEAAAK

SEQ ID NO: 146 - protein, 15 aa, Artificial sequence
  Synthetic sequence
  MISC_FEATURE: Linker
EAAAKEAAAKEAAAK

SEQ ID NO: 147 - protein, 20 aa, Artificial sequence
  Synthetic sequence
  MISC_FEATURE: Linker
EAAAKEAAAKEAAAKEAAAK

SEQ ID NO: 148 - protein, 25 aa, Artificial sequence
  Synthetic sequence
  MISC_FEATURE: Linker
EAAAKEAAAKEAAAKEAAAKEAAAK

SEQ ID NO: 149 - protein, 15 aa, Artificial sequence
  Synthetic sequence
  MISC_FEATURE: Linker
PSRLEEELRRRLTEP

SEQ ID NO: 150 - protein, 8 aa, Artificial sequence
  Synthetic sequence
  MISC_FEATURE: Linker
SACYCELS

SEQ ID NO: 151 - protein, 16 aa, Artificial sequence
  Synthetic sequence
  MISC_FEATURE: Linker
TQKSLSLSPGKGLGGL

SEQ ID NO: 152 - protein, 13 aa, Artificial sequence
  Synthetic sequence
  MISC_FEATURE: Linker
SLSLSPGKGLGGL

SEQ ID NO: 153 - protein, 5 aa, Artificial sequence
  Synthetic sequence
  MISC_FEATURE: Linker
GPGPG

SEQ ID NO: 154 - protein, 36 aa, Artificial sequence
  Synthetic sequence
  MISC_FEATURE: Linker
GGSAGGSGSGSSGGSSGASGTGTAGGTGSGSGTGSG

SEQ ID NO: 155 - protein, 36 aa, Artificial sequence
  Synthetic sequence
  MISC_FEATURE: Linker
GGSGGGSEGGGSEGGGSEGGGSEGGGSEGGGSGGGS

SEQ ID NO: 156 - protein, 12 aa, Artificial sequence
  Synthetic sequence
  MISC_FEATURE: Dimerization unit
ELKTPLGDTTHT

SEQ ID NO: 157 - protein, 13 aa, Artificial sequence
  Synthetic sequence
  MISC_FEATURE: Linker
HCLGKWLGHPDKF

SEQ ID NO: 158 - protein, 29 aa, Artificial sequence
  Synthetic sequence
  MISC_FEATURE: Linker
AHSLERVCHCLGKWLGHPDKFVGITYALT

SEQ ID NO: 159 - protein, 14 aa, Artificial sequence
  Synthetic sequence
  MISC_FEATURE: Linker
NTWTTCQSIAFPSK

SEQ ID NO: 160 - protein, 30 aa, Artificial sequence
  Synthetic sequence
  MISC_FEATURE: Linker
AVPVYIYFNTWTTCQSIAFPSKTSASIGSL

SEQ ID NO: 161 - protein, 21 aa, Artificial sequence
  Synthetic sequence
VHFFKNIVTPRTPPPSQGKGR

SEQ ID NO: 162 - protein, 36 aa, Artificial sequence
  Synthetic sequence
  MISC_FEATURE: Linker
RTQDENPVVHFFKNIVTPRTPPPSQGKGRGLSLSRF

SEQ ID NO: 163 - protein, 5 aa, Artificial sequence
  Synthetic sequence
  MISC_FEATURE: Linker
GLSGL

SEQ ID NO: 164 - protein, 5 aa, Artificial sequence
  Synthetic sequence
  MISC_FEATURE: Linker, REPEAT m is 1 or more
  MISC_FEATURE (1)..(5): The sequence can be repeated m times, where m is an integer greater than or equal to 1
GGGGS

SEQ ID NO: 165 - protein, 5 aa, Artificial sequence
  Synthetic sequence
  MISC_FEATURE: Linker, REPEAT m is 1 or more
  MISC_FEATURE (1)..(5): The sequence can be repeated m times, where m is an integer greater than or equal to 1
EAAAK

SEQ ID NO: 166 - protein, 7 aa, Artificial sequence
  Synthetic sequence
  MISC_FEATURE: Linker, REPEAT m is 1 or more
  MISC_FEATURE (1)..(5): The sequence can be repeated m times, where m is an integer greater than or equal to 1
EAAAKGS

SEQ ID NO: 167 - protein, 17 aa, Artificial sequence
  Synthetic sequence
  MISC_FEATURE: Linker
GPSRLEEELRRRLTEPG

SEQ ID NO: 168 - protein, 12 aa, Artificial sequence
  Synthetic sequence
  MISC_FEATURE: Linker
HEYGAEALERAG

SEQ ID NO: 169 - protein, 160 aa, Artificial sequence
  Synthetic sequence
  MISC_FEATURE: Murine IL-10
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 170 - protein, 21 aa, Artificial sequence
  Synthetic sequence
  MISC_FEATURE: Natural leader sequence murine SG3A2
MKLVSIFLLVTIGICGYSATA

SEQ ID NO: 171 - protein, 70 aa, Artificial sequence
  Synthetic sequence
  MISC_FEATURE: Murine SG3A2
LLINRLPVVDKLPVPLDDIIPSFDPLKMLLKTLGISVEHLVTGLKKCVDELGPEASEAVKKLLEALSHLV

SEQ ID NO: 172 - protein, 22 aa, Artificial sequence
  Synthetic sequence
  MISC_FEATURE: Natural leader sequence murine VSIG-3
MTRRRSAPASWLLVSLLGVATS

SEQ ID NO: 173 - protein, 218 aa, Artificial sequence
  Synthetic sequence
  MISC_FEATURE: Murine VSIG-3 Extracellular domain
LEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSV

SEQ ID NO: 174 - protein, 35 aa, Artificial sequence
  Synthetic sequence
  MISC_FEATURE: Natural leader sequence murine CTLA4
MACLGLRRYKAQLQLPSRTWPFVALLTLLFIPVFS

SEQ ID NO: 175 - protein, 126 aa, Artificial sequence
  Synthetic sequence
  MISC_FEATURE: Murine CTLA-4 Extracellular domain
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 176 - protein, 29 aa, Artificial sequence
  Synthetic sequence
  MISC_FEATURE: Natural leader sequence murine TGF(beta)1
MPPSGLRLLPLLLPLPWLLVLTPGRPAAG

SEQ ID NO: 177 - protein, 361 aa, Artificial sequence
  Synthetic sequence
  MISC_FEATURE: Murine TGF(beta)1 proprotein
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 178 - protein, 24 aa, Artificial sequence
  Synthetic sequence
  MISC_FEATURE: Natural leader sequence murine PD-1
MWVRQVPWSFTWAVLQLSWQSGWL

SEQ ID NO: 179 - protein, 145 aa, Artificial sequence
  Synthetic sequence
  MISC_FEATURE: Murine PD-1 Extracellular domain
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 180 - protein, 21 aa, Artificial sequence
  Synthetic sequence
  MISC_FEATURE: MOG(35-55)
MEVGWYRSPFSRVVHLYRNGK

SEQ ID NO: 181 - protein, 37 aa, Artificial sequence
  Synthetic sequence
  MISC_FEATURE: MOG(27-63)
SPGKNATGMEVGWYRSPFSRVVHLYRNGKDQDAEQAP

SEQ ID NO: 182 - protein, 37 aa, Artificial sequence
  Synthetic sequence
  MISC_FEATURE: MOG(27-63) with A/Q switch
SPGKNATGMEVGWYRSPFSRVVHLYRNGKDQDAEAQP

SEQ ID NO: 183 - protein, 20 aa, Artificial sequence
  Synthetic sequence
  MISC_FEATURE: GAD65(202-221)
TNMFTYEIAPVFVLLEYVTL

SEQ ID NO: 184 - protein, 274 aa, Artificial sequence
  Synthetic sequence
  MISC_FEATURE: Met e 1
MKLEKDNAMDRADTLEQQNKEANNRAEKSEEEVHNLQKRMQQLENDLDQVQESLLKANNQLVEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 185 - protein, 20 aa, Artificial sequence
  Synthetic sequence
  MISC_FEATURE: Met e 1 (16-35)
EQQNKEANNRAEKSEEEVHN

SEQ ID NO: 186 - protein, 20 aa, Artificial sequence
  Synthetic sequence
  MISC_FEATURE: Met e 1 (46-65)
DLDQVQESLLKANNQLVEKD

SEQ ID NO: 187 - protein, 20 aa, Artificial sequence
  Synthetic sequence
  MISC_FEATURE: Met e 1 (76-95)
AALNRRIQLLEEDLERSEER

SEQ ID NO: 188 - protein, 20 aa, Artificial sequence
  Synthetic sequence
  MISC_FEATURE: Met e 1 (136-155)
NQLKEARFLAEEADRKYDEV

SEQ ID NO: 189 - protein, 21 aa, Artificial sequence
  Synthetic sequence
  MISC_FEATURE: Met e 1 (210-230)
AYKEQIKTLTNKLKAAEARAE

SEQ ID NO: 190 - protein, 20 aa, Artificial sequence
  Synthetic sequence
  MISC_FEATURE: Met e 1 (241-260)
KEVDRLEDELVNEKEKYKSI

SEQ ID NO: 191 - protein, 284 aa, Artificial sequence
  Synthetic sequence
  MISC_FEATURE: Pan b 1
MDAIKKKMQAMKLEKDNAMDRADTLEQQNKEANNRAEKSEEEVFGLQKKLQQLENDLDSVQEALLKANQHLEEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 192 - protein, 20 aa, Artificial sequence
  Synthetic sequence
  MISC_FEATURE: Pan b 1 (26-45)
EQQNKEANNRAEKSEEEVFG

SEQ ID NO: 193 - protein, 20 aa, Artificial sequence
  Synthetic sequence
  MISC_FEATURE: Pan b 1 (56-75)
DLDSVQEALLKANQHLEEKD

SEQ ID NO: 194 - protein, 20 aa, Artificial sequence
  Synthetic sequence
  MISC_FEATURE: Pan b 1 (86-105)
AALNRRIQLLEEDLERSEER

SEQ ID NO: 195 - protein, 20 aa, Artificial sequence
  Synthetic sequence
  MISC_FEATURE: Pan b 1 (146-165)
NQLKEARFLAEEADRKYDEV

SEQ ID NO: 196 - protein, 21 aa, Artificial sequence
  Synthetic sequence
  MISC_FEATURE: Pan b 1 (220-240)
AYKEQIKTLTNKLKAAEARAE

SEQ ID NO: 197 - protein, 20 aa, Artificial sequence
  Synthetic sequence
  MISC_FEATURE: Pan b 1 (251-270)
KEVDRLEDELVNEKEKYKSI

SEQ ID NO: 198 - protein, 23 aa, Artificial sequence
  Synthetic sequence
  MISC_FEATURE: Natural leader sequence humane CCL3L1
MQVSTAALAVLLCTMALCNQVLS

SEQ ID NO: 199 - protein, 70 aa, Artificial sequence
  Synthetic sequence
  MISC_FEATURE: humane CCL3L1
APLAADTPTACCFSYTSRQIPQNFIADYFETSSQCSKPSVIFLTKRGRQVCADPSEEWVQKYVSDLELSA

SEQ ID NO: 200 - DNA, 36 bp, Artificial sequence
  Synthetic sequence
  misc_feature: Hinge-region 1 from human IgG3
gagctcaaaaccccacttggtgacacaactcacaca

SEQ ID NO: 201 - DNA, 30 bp, Artificial sequence
  Synthetic sequence
  misc_feature: Hinge-region 4 from human IgG3
ggcggtggaagcagcggaggtggaagtgga

SEQ ID NO: 202 - protein, 107 aa, Artificial sequence
  Synthetic sequence
  MISC_FEATURE: Human IgG3 CH3 domain
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 203 - DNA, 321 bp, Artificial sequence
  Synthetic sequence
  misc_feature: Human IgG3 CH3 domain
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 204 - DNA, 15 bp, Artificial sequence
  Synthetic sequence
  misc_feature: Linker
ggcctcggtggcctg

SEQ ID NO: 205 - protein, 390 aa, Artificial sequence
  Synthetic sequence
  MISC_FEATURE: Human transforming growth factor beta-1
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 206 - protein, 442 aa, Artificial sequence
  Synthetic sequence
  MISC_FEATURE: Human Transforming growth factor beta-2
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 207 - protein, 309 aa, Artificial sequence
  Synthetic sequence
  MISC_FEATURE: Human transforming growth factor beta-3
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 208 - DNA, 1173 bp, Artificial sequence
  Synthetic sequence
  misc_feature: Human transforming growth factor beta-1
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 209 - DNA, 1329 bp, Artificial sequence
  Synthetic sequence
  misc_feature: Human transforming growth factor beta 2
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 210 - DNA, 930 bp, Artificial sequence
  Synthetic sequence
  misc_feature: Human transforming growth factor beta 3
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtga

SEQ ID NO: 211 - protein, 178 aa, Artificial sequence
  Synthetic sequence
  MISC_FEATURE: Human interleukin-10 isoform 1
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 212 - DNA, 537 bp, Artificial sequence
  Synthetic sequence
  misc_feature: Human interleukin 10 (IL10)
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 213 - protein, 93 aa, Artificial sequence
  Synthetic sequence
  MISC_FEATURE: Human secretoglobin family 3A member 2 precursor (SCGB3A2)
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 214 - DNA, 282 bp, Artificial sequence
  Synthetic sequence
  misc_feature: Human secretoglobin family 3A member 2 (SCGB3A2)
atgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtga

SEQ ID NO: 215 - protein, 241 aa, Artificial sequence
  Synthetic sequence
  MISC_FEATURE: IGSF11 (VSIG3) (ECD)
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 216 - DNA, 723 bp, Artificial sequence
  Synthetic sequence
  misc_feature: IGSF11 (VSIG3) (ECD)
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgga

SEQ ID NO: 217 - protein, 161 aa, Artificial sequence
  Synthetic sequence
  MISC_FEATURE: CTLA4_HUMAN Cytotoxic T-lymphocyte protein 4 (ECD)
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 218 - DNA, 483 bp, Artificial sequence
  Synthetic sequence
  misc_feature: Human cytotoxic T-lymphocyte associated protein 4 (CTLA4) (ECD)
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgac

SEQ ID NO: 219 - protein, 170 aa, Artificial sequence
  Synthetic sequence
  MISC_FEATURE: Human programmed cell death protein 1 PDCD1 (PD-1) (ECD)
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 220 - DNA, 510 bp, Artificial sequence
  Synthetic sequence
  misc_feature: Human programmed cell death 1 PDCD1 (PD-1) (ECD)
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg